(19) United States
(12) Patent Application Publication
(10) Pub. No.: US 2017/0131280 A1
(43) Pub. Date: May 11, 2017

(54) METHODS, ASSAYS, AND SYSTEMS RELATING TO SAKT

(71) Applicant: THE GENERAL HOSPITAL CORPORATION, Boston, MA (US)

(72) Inventors: David SCADDEN, Weston, MA (US); Dongjun LEE, Cambridge, MA (US)

(73) Assignee: THE GENERAL HOSPITAL CORPORATION, Boston, MA (US)

(21) Appl. No.: 15/273,783

(22) Filed: Sep. 23, 2016

Related U.S. Application Data
(63) Continuation of application No. 14/374,434, filed on Jul. 24, 2014, now abandoned.
(60) Provisional application No. 61/600,733, filed on Feb. 20, 2012.

(51) Int. Cl. G01N 33/574; C12N 15/113; C12Q 1/68; G01N 33/50

(52) U.S. Cl. CPC G01N 33/5748; G01N 33/5091; C12N 15/1135; C12Q 1/6886; C12N 2310/14; C12Q 2600/158; C12Q 2600/106; G01N 2440/14; G01N 2333/705

(57) ABSTRACT

The technology described herein is directed to methods, assays, and systems relating to determining the level of SAKT signaling activity in a sample obtained from a subject as well as methods relating to administering inhibitors and/or agonists of SAKT signaling.

### CROSS-REFERENCE TO RELATED APPLICATIONS

This Application is a continuation of co-pending U.S. application Ser. No. 14/374,434 filed on Jul. 24, 2014, which is a 35 U.S.C. §371 National Phase Entry Application of International Application No. PCT/US2013/026794 filed Feb. 20, 2013, which designates the U.S., and which claims the benefit under 35 U.S.C. §119(e) of U. S. Provisional Application No. 61/600,733, filed Feb. 20, 2012, the contents of each of which are incorporated herein by reference in their entireties.

### GOVERNMENT SUPPORT

This invention was made with federal funding under Grant Nos. U01HL100402, HL97794, and HL044851 awarded by the National Institutes of Health and the National Heart, Lung and Blood Institute. The U.S. government has certain rights in the invention.

### SEQUENCE LISTING

The instant application contains a Sequence Listing which has been submitted in ASCII format via EFS-Web and is hereby incorporated by reference in its entirety. Said ASCII copy, created on Feb. 18, 2013, is named 030258-073501-PCT SL.txt and is 117,611 bytes in size.

### TECHNICAL FIELD

The technology described herein relates to SAKT signaling activity and the treatment of disorders, e.g. cancer and ischemic injury.

### BACKGROUND

The AKT kinase is central to cellular proliferative, survival and metabolic responses. Inappropriate activation of AKT is among the most common alterations in human cancer. For example, in acute myeloid leukemia (AML), which is characterized by a high rate of relapse and a five year survival rate of only 20% (Rowe. Res Clin Haematol. 2008 21:1-3), nearly all subjects show an abnormal level of AKT activity.

### SUMMARY

Described herein is the inventors' discovery of a novel regulator of the AKT-FOXO signaling axis. This novel regulator, termed Suppressor of AKT (SAKT), is a transmembrane receptor which inhibits mTORC2 by suppressing the activity of the RICTOR subunit of mTORC2. This suppression of RICTOR causes a decrease in the level of, e.g. AKT phosphorylated at 5473 and an increase in FOXO. The activity of SAKT is specific for control of AKT via phosphorylation at S473 and signaling through FOXO and does not regulate signaling through other AKT targets.

This discovery has important therapeutic and diagnostic applications. For instance, AKT signaling is perturbed in many cancers. In the case of AML, approximately 60% of patients will exhibit increased levels of phosphorylated AKT (as is exhibited in many epithelial cancers), while 40% of AML patients will exhibit decreased levels of phosphorylated AKT and increased levels of FOXO activity. The idenitification of SAKT, as described herein, as a modulator of this activity which is perturbed in cancer, permits therapeutic approaches to treating cancer.

Furthermore, as demonstrated herein, SAKT signaling activity can control cell survival, as opposed to cell proliferation, providing a means for improving the survival of cells that have been injured, e.g. cells which have experienced ischemic injury, e.g. as a result of cardiac infarction or stroke.

Additionally, certain growth factors are known to act via AKT signaling. Accordingly, provided herein are methods of modulating the sensitivity of a cell to a growth factor, e.g. by contacting the cell with an antagonist or agonist of SAKT signaling in order to render the sell more or less (respectively) sensitive to the growth factor.

In one aspect, described herein is a method of treating a subject having cancer, the method comprising: determining, in a cancer cell sample obtained from the subject, the level of SAKT signaling activity; and administering a treatment to the subject; wherein a subject with a decreased level of SAKT signaling activity is administered a treatment comprising a therapeutically effective amount of an agonist of SAKT signaling; and wherein a subject with an increased level of SAKT signaling activity, as compared to a reference level, is administered a treatment comprising a therapeutically effective amount of an inhibitor of SAKT signaling. In one aspect, described herein is a method of determining whether a cancer patient would benefit from treatment with an agonist of SAKT signaling, the method comprising: determining the level of SAKT signaling activity in a cancer cell sample obtained from the subject; wherein an agonist of SAKT signaling is indicated as an appropriate treatment if the level of SAKT signaling activity is decreased relative to a reference; and wherein an agonist of SAKT signaling is not indicated as an appropriate treatment if the level of SAKT signaling activity is not decreased relative to a reference. In one aspect, described herein is a method of determining whether a cancer patient would benefit from treatment with an inhibitor of SAKT signaling, the method comprising: determining the level of SAKT signaling activity in a cancer cell sample obtained from the subject; wherein an inhibitor of SAKT signaling is indicated as an appropriate treatment if the level of SAKT signaling is increased relative to a reference; and wherein an inhibitor of SAKT signaling is not indicated as an appropriate treatment if the level of SAKT signaling activity is not increased relative to a reference.

In some embodiments, increased SAKT signaling activity can be determined by detecting a decreased level of AKT expression products and, optionally, an increased level of FOXO expression product. In some embodiments, decreased SAKT signaling activity can be determined by detecting an increased level of AKT expression products. In some embodiments, the AKT expression products can comprise phosphorylated AKT expression products. In some embodiments, the AKT expression products can consist of phosphorylated AKT expression products. In some embodiments, increased SAKT signaling activity can be determined by detecting a marker selected from the group consisting of: decreased levels of RICTOR; decreased levels of FOXO phosphorylation; decreased levels of FOXO3 phosphorylated at S253 and/or T32; decreased levels of mTOR phosphorylated at S2448; decreased levels of S6 phosphorylated at S235 and/or S236, decreased levels of 4EBP1 phosphorylated at T37 and/or T46; decreased levels of PRAS40 phosphorylated at T246; decreased levels of phosphorylated STAT3; decreased levels of phosphorylated SGK; decreased levels of SGK phosphorylated at S422; decreased levels of phosphorylated PKCα; decreased levels of PKCα phosphorylated at S638; and decreased levels of AKT phosphorylated at S473. In some embodiments, decreased SAKT signaling activity can be determined by detecting a marker selected from the group consisting of: increased levels of RICTOR; increased levels of FOXO phosphorylation; increased levels of FOXO3 phosphorylated at S253 and/or T32; increased levels of mTOR phosphorylated at S2448; increased levels of S6 phosphorylated at S235 and/or S236; increased levels of 4EBP1 phosphorylated at T37 and/or T46; increased levels of PRAS40 phosphorylated at T246; increased levels of phosphorylated STAT3; increased levels of phosphorylated SGK; increased level of SGK phosphorylated at S422; increased levels of phosphorylated PKCα; increased levels of PKCα phosphorylated at S638; and increased levels of AKT phosphorylated at S473.

In some embodiments, the agonist of SAKT signaling can be selected from the group consisting of an agonist of SAKT; an inhibitor of RICTOR; an inhibitor of mTORC2; and an inhibitor of RICTOR-mTORC2 interaction. In some embodiments, the agonist of SAKT can be selected from the group consisting of: an antibody reagent agonist; and a nucleic acid encoding SAKT. In some embodiments, the inhibitor of SAKT signaling can be selected from the group consisting of: an inhibitor of SAKT; an agonist of RICTOR; an agonist of mTORC2; and an agonist of RICTOR-mTORC2 interaction. In some embodiments, the inhibitor of SAKT can be selected from the group consisting of: an inhibitory nucleic acid molecule; and an antibody reagent.

In some embodiments, the cell can be selected from the group consisting of: a hematopoietic cancer cell and an epithelial cancer cell. In some embodiments, the epithelial cancer cell can be selected from the group consisting of: carcinoma; adenocarcinoma; basal cell carcinoma; squamous cell carcinoma; large cell carcinoma; small cell carcinoma; colorectal adenocarcinoma; lung cancer; breast cancer; prostate cancer; colon cancer; rectal cancer; pancreatic cancer; kidney cancer; ovarian cancer; stomach cancer; intestinal cancer; oral cancer; esophageal cancer; lip cancer; bladder cancer; cervical cancer; skin cancer; hepatocellular carcinoma; and renal cell carcinoma.

In some embodiments, the level of AKT or Foxo expression products can be determined by measuring the level of RNA transcripts. In some embodiments, the RNA transcript level can be measured using reverse transcription polymerase chain reaction (RT-PCR). In some embodiments, the level of AKT and Foxo expression products can be determined by measuring the level of polypeptides. In some embodiments, the polypeptide level can be measured using immunochemistry. In some embodiments, the immunochemical method can comprise: contacting a biofluid test sample obtained from a subject with a detectable anti-AKT antibody reagent and optionally, an anti-Foxo antibody reagent; and detecting the presence or intensity of a detectable signal; wherein the expression level of AKT polypeptide, and optionally, Foxo polypeptide, is indicated by the level of the detectable signal. In some embodiments, the antibody reagent can be detectably labeled or capable of generating a detectable signal.

In some embodiments, the sample can comprise a material selected from the group consisting of: blood or a product thereof; serum; plasma; and a tumor biopsy. In some embodiments, the level of a marker of SAKT signaling activity, can be normalized relative to the expression level of one or more reference genes or reference proteins. In some embodiments, the reference expression level can be the expression level in a sample obtained from a subject not having cancer. In some embodiments, the reference expression level can be the expression level in a prior sample obtained from the subject. In some embodiments, an increased level can be a level at least 25% greater than a reference level. In some embodiments, a decreased level can be a level at least 25% less than a reference level. In some embodiments, the expression level of no more than 20 other genes is determined. In some embodiments, the expression level of no more than 10 other genes is determined. In some embodiments, the subject can be a human.

In one aspect, described herein is an assay comprising contacting a cancer cell sample obtained from a subject with a detectable anti-AKT antibody reagent; and detecting the presence or intensity of a detectable signal; wherein an increase in the level of AKT polypeptide, indicated by the level of the detectable signal, relative to a reference level indicates the subject is in need of treatment with an agonist of SAKT signaling activity; and wherein a decrease in the level of AKT polypeptide, indicated by the level of the detectable signal, relative to a reference level indicates the subject is in need of treatment with an inhibitor of SAKT signaling activity. In some embodiments, the method can further comprise contacting the cancer cell sample with a detectable anti-Foxo antibody reagent In some embodiments, the anti-AKT antibody reagent can be specific for AKT polyeptide phosphorylated at S473.

In some embodiments, the agonist of SAKT signaling can be selected from the group consisting of: an agonist of SAKT; an inhibitor of RICTOR; an inhibitor of mTORC2; and an inhibitor of RICTOR-mTORC2 interaction. In some embodiments, the agonist of SAKT signaling can be selected from the group consisting of an agonist of SAKT; an inhibitor of RICTOR; an inhibitor of mTORC2; and an inhibitor of RICTOR-mTORC2 interaction. In some embodiments, the agonist of SAKT can be selected from the group consisting of: an antibody reagent agonist; and a nucleic acid encoding SAKT. In some embodiments, the inhibitor of SAKT signaling can be selected from the group consisting of: an inhibitor of SAKT; an agonist of RICTOR; an agonist of mTORC2; and an agonist of RICTOR-mTORC2 interaction. In some embodiments, the inhibitor of SAKT can be selected from the group consisting of: an inhibitory nucleic acid molecule; and an antibody reagent.

In some embodiments, the cell can be selected from the group consisting of: a hematopoietic cancer cell and an epithelial cancer cell. In some embodiments, the epithelial cancer cell can be selected from the group consisting of: carcinoma; adenocarcinoma; basal cell carcinoma; squamous cell carcinoma; large cell carcinoma; small cell carcinoma; colorectal adenocarcinoma; lung cancer; breast cancer; prostate cancer; colon cancer; rectal cancer; pancreatic cancer; kidney cancer; ovarian cancer; stomach cancer; intestinal cancer; oral cancer; esophageal cancer; lip cancer; bladder cancer; cervical cancer; skin cancer; hepatocellular carcinoma; and renal cell carcinoma.

In some embodiments, the sample can comprise a material selected from the group consisting of: blood or a product thereof; serum; plasma; and a tumor biopsy. In some embodiments, the level of a marker of SAKT signaling activity, can be normalized relative to the expression level of one or more reference genes or reference proteins. In some embodiments, the reference expression level can be the expression level in a sample obtained from a subject not having cancer. In some embodiments, the reference expression level can be the expression level in a prior sample obtained from the subject. In some embodiments, an increased level can be a level at least 25% greater than a reference level. In some embodiments, a decreased level can be a level at least 25% less than a reference level. In some embodiments, the expression level of no more than 20 other genes is determined. In some embodiments, the expression level of no more than 10 other genes is determined. In some embodiments, the subject can be a human.

In one aspect, described herein is a method of suppressing AKT activity, comprising administering an agonist of SAKT activity or expression. In one aspect, described herein is a method of treating ischemic injury, the method comprising administering an agonist of SAKT signaling activity. In one aspect, described herein is method of altering the sensitivity of a cell to a growth factor, the method comprising; contacting the cell with an inhibitor of SAKT signaling activity to render the cell more sensitive to the growth factor; or contacting the cell with an agonist of SAKT signaling activity to render the cell less sensitive to the growth factor. In some embodiments, the growth factor can be selected from the group consisting of: insulin; epidermal growth factor (EGF); hematopoietic growth factor; vascular endothelial growth factor (VEGF); insulin-like growth factor-1 (IGF); platelet-derived growth factor (PDGF); granulocyte colony-stimulating factor (G-CSF); platelet activating factor; and macrophage stimulating factor. In some embodiments, the agonist or inhibitor of SAKT signaling activity can be administered to a subject. In some embodiments, the subject can be in need of treatment for a condition selected from the group consisting of insulin resistance; diabetes; cancer; proliferative diseases; and wound healing. In some embodiments, the agonist of SAKT signaling can be selected from the group consisting of: an agonist of SAKT; an inhibitor of RICTOR; an inhibitor of mTORC2; and an inhibitor of RICTOR-mTORC2 interaction. In some embodiments, the agonist of SAKT signaling can be selected from the group consisting of an agonist of SAKT; an inhibitor of RICTOR; an inhibitor of mTORC2; and an inhibitor of RICTOR-mTORC2 interaction. In some embodiments, the agonist of SAKT can be selected from the group consisting of: an antibody reagent agonist; and a nucleic acid encoding SAKT. In some embodiments, the inhibitor of SAKT signaling can be selected from the group consisting of: an inhibitor of SAKT; an agonist of RICTOR; an agonist of mTORC2; and an agonist of RICTOR-mTORC2 interaction. In some embodiments, the inhibitor of SAKT can be selected from the group consisting of: an inhibitory nucleic acid molecule; and an antibody reagent.

In one aspect, described herein is a computer system for determining the appropriate treatment for a subject having cancer, the system comprising: a measuring module configured to measure the level of SAKT signaling activity, in a test sample obtained from a subject; a storage module configured to store output data from the determination module; a comparison module adapted to compare the data stored on the storage module with a reference level, and to provide a retrieved content, and a display module for displaying whether the sample comprises a level of SAKT signaling activity which is significantly increased or decreased relative to the reference level and/or displaying the relative SAKT signaling activity. In some embodiments, the measuring module can measure the intensity of a detectable signal from an assay indicating the level of a polypeptide marker of SAKT signaling activity in the test sample. In some embodiments, the assay can be an immunoassay. In some embodiments, if the computing module determines that the level of SAKT signaling activity in the test sample obtained from a subject is less by a statistically significant amount than the reference level, the display module can display a signal indicating that the expression levels in the sample obtained from a subject are less than those of the reference level. In some embodiments, if the computing module determines that the level of SAKT signaling activity in the test sample obtained from a subject is less by a statistically significant amount than the reference level, the display module can display a signal indicating that the appropriate treatment for the subject is an agonist of SAKT signaling. In some embodiments, if the computing module determines that the level of SAKT signaling activity in the test sample obtained from a subject is greater by a statistically significant amount than the reference level, the display module can display a signal indicating that the expression levels in the sample obtained from a subject are greater than those of the reference level. In some embodiments, if the computing module determines that the level of SAKT signaling activity in the test sample obtained from a subject is greater by a statistically significant amount than the reference level, the display module can display a signal indicating that the appropriate treatment for the subject is an inhibitor of SAKT signaling. In some embodiments, the signal can indicate the degree to which the level of SAKT signaling activity in the sample obtained from a subject varies from the reference level. In some embodiments, increased SAKT signaling activity can be determined by detecting a decreased level of AKT expression products and, optionally, an increased level of FOXO expression product. In some embodiments, decreased SAKT signaling activity can be determined by detecting an increased level of AKT expression products.

In some embodiments, increased SAKT signaling activity can be determined by detecting a decreased level of AKT expression products and, optionally, an increased level of FOXO expression product. In some embodiments, decreased SAKT signaling activity can be determined by detecting an increased level of AKT expression products. In some embodiments, the AKT expression products can comprise phosphorylated AKT expression products. In some embodiments, the AKT expression products can consist of phosphorylated AKT expression products. In some embodiments, increased SAKT signaling activity can be determined by detecting a marker selected from the group consisting of: decreased levels of RICTOR; decreased levels of FOXO phosphorylation; decreased levels of FOXO3 phosphorylated at S253 and/or T32; decreased levels of mTOR phosphorylated at S2448; decreased levels of S6 phosphorylated at S235 and/or S236, decreased levels of 4EBP1 phosphorylated at T37 and/or T46; decreased levels of PRAS40 phosphorylated at T246; decreased levels of phosphorylated STAT3; decreased levels of phosphorylated SGK; decreased levels of SGK phosphorylated at S422; decreased levels of phosphorylated PKCα; decreased levels of PKCα phosphorylated at S638; and decreased levels of AKT phosphorylated at S473. In some embodiments, decreased SAKT signaling activity can be determined by detecting a marker selected from the group consisting of: increased levels of RICTOR; increased levels of FOXO phosphorylation; increased levels of FOXO3 phosphorylated at S253 and/or T32; increased levels of mTOR phosphorylated at S2448; increased levels of S6 phosphorylated at S235 and/or S236; increased levels of 4EBP1 phosphorylated at T37 and/or T46; increased levels of PRAS40 phosphorylated at T246; increased levels of phosphorylated STAT3; increased levels of phosphorylated SGK; increased level of SGK phosphorylated at S422; increased levels of phosphorylated PKCα; increased levels of PKCα phosphorylated at S638; and increased levels of AKT phosphorylated at S473.

In some embodiments, the agonist of SAKT signaling can be selected from the group consisting of an agonist of SAKT; an inhibitor of RICTOR; an inhibitor of mTORC2; and an inhibitor of RICTOR-mTORC2 interaction. In some embodiments, the agonist of SAKT can be selected from the group consisting of: an antibody reagent agonist; and a nucleic acid encoding SAKT. In some embodiments, the inhibitor of SAKT signaling can be selected from the group consisting of: an inhibitor of SAKT; an agonist of RICTOR; an agonist of mTORC2; and an agonist of RICTOR-mTORC2 interaction. In some embodiments, the inhibitor of SAKT can be selected from the group consisting of: an inhibitory nucleic acid molecule; and an antibody reagent.

In some embodiments, the cell can be selected from the group consisting of: a hematopoietic cancer cell and an epithelial cancer cell. In some embodiments, the epithelial cancer cell can be selected from the group consisting of: carcinoma; adenocarcinoma; basal cell carcinoma; squamous cell carcinoma; large cell carcinoma; small cell carcinoma; colorectal adenocarcinoma; lung cancer; breast cancer; prostate cancer; colon cancer; rectal cancer; pancreatic cancer; kidney cancer; ovarian cancer; stomach cancer; intestinal cancer; oral cancer; esophageal cancer; lip cancer; bladder cancer; cervical cancer; skin cancer; hepatocellular carcinoma; and renal cell carcinoma.

In some embodiments, the level of AKT or Foxo expression products can be determined by measuring the level of RNA transcripts. In some embodiments, the RNA transcript level can be measured using reverse transcription polymerase chain reaction (RT-PCR). In some embodiments, the level of AKT and Foxo expression products can be determined by measuring the level of polypeptides. In some embodiments, the polypeptide level can be measured using immunochemistry. In some embodiments, the immunochemical method can comprise: contacting a biofluid test sample obtained from a subject with a detectable anti-AKT antibody reagent and optionally, an anti-Foxo antibody reagent; and detecting the presence or intensity of a detectable signal; wherein the expression level of AKT polypeptide, and optionally, Foxo polypeptide, is indicated by the level of the detectable signal. In some embodiments, the antibody reagent can be detectably labeled or capable of generating a detectable signal.

In some embodiments, the sample can comprise a material selected from the group consisting of: blood or a product thereof; serum; plasma; and a tumor biopsy. In some embodiments, the level of a marker of SAKT signaling activity, can be normalized relative to the expression level of one or more reference genes or reference proteins. In some embodiments, the reference expression level can be the expression level in a sample obtained from a subject not having cancer. In some embodiments, the reference expression level can be the expression level in a prior sample obtained from the subject. In some embodiments, an increased level can be a level at least 25% greater than a reference level. In some embodiments, a decreased level can be a level at least 25% less than a reference level. In some embodiments, the expression level of no more than 20 other genes is determined. In some embodiments, the expression level of no more than 10 other genes is determined.

### BRIEF DESCRIPTION OF THE DRAWINGS

FIGS. 1A-1E demonstrate that SAKT regulates AKT-FoxO-mTOR signaling in BM cells. FIG. 1A depicts the results of western blot analysis (using the indicated antibodies) of BM cells from overexpression (left panels) and depletion (right panels) of SAKT. Beta-Actin (β-Actin) was used as a loading control. Overexpression of SAKT was performed after transduction of BM cells by second spinoculation, followed by sorting GFP⁺ cells, and depletion of SAKT was performed after transduction of BM cells by second spinoculation, followed by puromycin selection for 48-72 hours. FIG. 1B depicts a graph of the fold change of the indicated proteins ((pAKT^{S473}, Rictor and pSTAT3^{Y705})/β-Actin, and (pAKT^{S473}/AKT and pSTAT3^{Y705}/STAT3) ratios normalized to that in the numbers below FIG. 1A. Data are means±s.e.m. (n≧3). FIG. 1C depicts the results of experiments in which 293T cells transfected with vectors encoding either Flag-SAKT full length or Flag-SAKT ΔC mutant were lysed and subjected to IP using an antibody directed against Flag and IgG, respectively. The resulting precipitates, and the corresponding whole-cell lysates (WCL), were subjected to western blot analysis using the indicated antibodies. FIG. 1D depicts the results of immunoblots from experiments in which SAKT, Rictor or STAT3 were immunoprecipitated from BM cells, and subjected to western blot analysis using the indicated antibodies. FIG. 1E depicts the results of immunoblots from experiments in which Rictor or SAKT was immunoprecipitated from 293T (upper) and BM (lower) cells, and subjected to western blot analysis using the indicated antibodies.

FIGS. 2A-2E demonstrate that SAKT interact with mTORC2 and STAT3, and regulates their activity in BM cells. FIG. 2A depicts immunoblot analyses for the presence of the indicated components of the mTORC2 in immunoprecipitates prepared from BM cell lysates with antibodies against SAKT, RICTOR, RAPTOR, mTOR, or mLST8. FIG. 2B depicts RICTOR or mTOR immunoprecipitates were prepared from control, 293T cells transfected with vectors encoding Flag-SAKT full length or stimulated (+insulin) 293T cells transfected with vectors encoding Flag-SAKT full length, and their ability to phosphorylate purified Akt at S473 was determined. An immunoblot for the levels of RICTOR, mTOR and mLST8 in each immuoprecipitate is shown. FIG. 2C depicts immunochemical results of wild-type and Rictor KO BM cells expressing shRNA constructs against control or SAKT starved for 3 hr, and then restimulated insulin (1 μg/mL) or serum (10% serum), respectively, for 30 min prior to lysis, and analyzed by western blotting (n=3 mice pool per each genotype). FIG. 2D depicts the immunochemical results of experiments in which RICTOR or SAKT were immunoprecipitated from wild-type and Rictor KO BM cells, and subjected to western blot analysis using the indicated antibodies. FIG. 2E depicts immunochemical results of wild-type and Rictor KO BM cells expressing shRNA constructs against control or Sakt, and subjected to western blot analysis using the indicated antibodies.

FIGS. 3A-3E demonstrate that FoxO3 activates SAKT expression in BM cells. FIG. 3A depicts a schematic representation of gene expression assay in BM cells. FIG. 3B depicts a graph of quantitative RT-PCR for SAKT expression. Data are means±s.e.m. (n≧6). **p<0.01. FIG. 3C depicts a graph of SAKT expression in BM cells from wild-type and FoxO1/3/4 KO mice. Data are means±s.e.m. (n=3 mice per each genotype). **p<0.01. FIG. 3D depicts a graph of ChIP analysis for Flag-FoxO3 (left), MIG-FoxO3 (right), or mock expressing NIH3T3 or BM cells that used the indicated antibodies, respectively. Data are means±s.e.m. (n≧3). **p<0.01. FIG. 3E depicts a graph of relative luciferase activity of FoxO3 on SAKT promoter. Data are means±s.e.m. (n=12). **p<0.01.

FIGS. 4A-4H demonstrate the physiological role of SAKT in BM cells. FIG. 4A depicts an experimental scheme for BMT experiments. FIG. 4B depicts a graph of the percent (donor GFP⁺ cells/pre-transplant GFP⁺ cells) from recipients as determined by fluorescence-activated cell sorting (FACS) at the indicated times. Data are expressed as mean s.e.m. (n=6). *p<0.05. **p<0.01. FIG. 4C depicts a graph of FACS analysis of the normalized percentage contributions in the BM cells of recipients at 20 weeks post-transplantation. Data are means±s.e.m. (n=6). *p<0.05. FIG. 4D depicts a graph of multilineage differentiation into B (B220⁺), T (CD3⁺) ,and myeloid lineages (Mac1⁺) among GFP⁺ cells at 20 weeks post-transplantation. (n≧4). Data are means±s.e.m. FIG. 4E depicts a graph of GFP⁺ cells sorted from BM cells of recipients and hematopoietic stem and progenitor (Lin⁻c-Kit⁺Sca1⁺) populations determined by FACS at 20 weeks post-transplantation. Data are means±s.e.m. (n=6). *p<0.05. FIG. 4F depicts a graph of the colony growth of transduced BM cells sorted, and plated in cytokine-supplemented methylcellulose medium. Data are means±s.e.m. (n≧6). *p<0.05. **p<0.01. FIG. 4G depicts a graph of cell growth. Transduced BM cells were placed into liquid culture in the presence of IL-3, IL-6, and stem cell factor, and counted GFP⁺ cells either everyday. Data are means±s.e.m. *p<0.05. FIG. 4H depicts a schematic in which SAKT suppresses phosphorylated AKT on S473 through the modulation of Rictor-dependent pathways in BM cells. Model represents SAKT elevation negatively impacts to Rictor-AKT signaling in BM cells.

FIGS. 5A-5B depict the characterization of cells shRNA-depleted for SAKT. FIG. 5A depicts the results of experiments in which BM cells from depletion of SAKT were analyzed by western blotting using the indicated antibodies (top panels) and RT-PCR (bottom panels). FIG. 5B depicts immunoblots demonstrating the restoration of SAKT-depleted BM cells with SAKT expression vector.

FIG. 6 depicts a graph demonstrating that SAKT regulates Rictor expression in BM cells. Quantitative RT-PCR (qRTPCR) for Rictor expression from overexpressing or shRNA-depleted for SAKT BM cells. Data are expressed as mean±s.e.m. (n=6). *p<0.05. **p<0.01.

FIG. 7 depicts a graph demonstrating that Rictor expression by perturbing SAKT was not affected by deletion of FoxO1/3/4. qRT-PCR for Rictor expression from overexpressing or shRNA-depleted for SAKT FoxO1/3/4 BM cells. Data are expressed as mean±s.e.m. (n=6).

FIG. 8 depicts immunoblot results from experiments in which 293T cells transfected with vectors encoding Flag-SAKT full length were lysed and subjected to IP using an antibody directed against Flag and IgG, respectively. The resulting precipitates, and the corresponding whole-cell lysates (WCL), were subjected to western blot analysis using the indicated antibodies.

FIGS. 9A-9B depict serum induces SAKT dephosphorylation. BM cells were starved in serum-free medium for 12 hr and then stimulated with 10% FBS or indicated cytokines for 48 hr (FIG. 9A) or different concentrations of Egf treatment for the indicated times (FIG. 9B).

FIG. 10 depicts a graph of multilineage differentiation into B (B220⁺), T (CD3⁺), and myeloid lineages (Mac1⁺) among GFP⁺ cells at before and at 20 weeks post-transplantation in the experiments depicted in FIG. 4A. (n≧4). Data are means±s.e.m.

FIGS. 11A-11C depict SAKT suppresses MLL-AF9-Induced Leukemia in vivo. FIG. 11A depicts the experimental scheme used in FIGS. 11B and 11C. FIG. 11B depicts a graph of the expression of Mil-Af9, Gapdh and Sakt using RT-PCR and gross anatomical view of spleens recovered from indicated MLL-AF9⁺ transplanted recipient mice. FIG. 11C depicts a graph of Kaplan-Meier survival curve analysis of mice transplanted as descrived above (Ctrl recipients vs. SAKT recipients *p=0.02, n=8).

FIG. 12 is a diagram of an exemplary embodiment of a system for performing an assay for determining the level of, e.g., SAKT signaling activity in sample obtained from a subject.

FIG. 13 is a diagram of an embodiment of a comparison module as described herein.

FIG. 14 is a diagram of an exemplary embodiment of an operating system and instructions for a computing system as described herein.

FIGS. 15A-15B demonstrate that Osx-GFP-Cre+; Dicer1floxed mice showed alterations in the AKT signaling pathway. FIG. 15A depicts the results of experiments in which stromal (left panels) and hematopoietic cells (right panels) from wild-type and Dicer1 KO mice were isolated, and analyzed by western blotting using the indicated antibodies. (n=2 or 3 mice pool per each genotype). FIG. 15B depicts a graph of the fold changes of indicated proteins ((pAKTS473 and Rictor)/β-Actin) ratios normalized to that in the numbers below the panel (15A).

### DETAILED DESCRIPTION

Embodiments of the technology described herein relate to the inventors' discovery of SAKT, a regulator of the AKT-FOXO signaling axis, which permits therapeutic and diagnostic methods relating to cancer and, e.g. ischemic injury or growth factor sensitivity.

As described herein, “Suppressor of AKT” or “SAKT” refers to a transmembrane protein which, as described herein, regulates AKT activity via RICTOR. As described herein, the inventors have discovered that this polypeptide, previously known as C14ORF37, inhibits signaling of the AKT-FOXO axis by inhibiting the RICTOR subunit of mTORC2. By inhibiting the activity of RICTOR, the phosphorylation of S473 of AKT is decreased, leading to decrease in AKT-mediated inhibition of FOXO polypeptides, which are a family of transcription factors. This pathway of SAKT-RICTOR(mTORC2)-AKT-FOXO-target gene transcription is referred to herein as the SAKT signaling pathway. In some embodiments, the activity of SAKT is specific for the AKT-FOXO axis, e.g. other targets of AKT (e.g. TSC2, or mTORC1) are not part of the SAKT signaling pathway and/or are not affected by an increase or decrease in SAKT activity. Thus, an increase in the activity of SAKT itself can result in a decreased amount of AKT (e.g. phosphorylated AKT and/or AKT activity) while a decrease in the activity of SAKT can result in an increased amount of AKT (e.g. phosphorylated AKT and/or AKT activity).

AKT signaling has been implicated in, e.g. cancer, cell survival, and the response to growth factors. Accordingly, the discovery of SAKT and the SAKT signaling pathway permits therapeutic and diagnostic approaches to conditions in which modulation of AKT activity would be beneficial, as described herein.

As described elsewhere herein, some cancers (e.g. many epithelial cancers and about 60% of AML's) exhibit increased AKT activity. As described herein, such cancers can be treated by administering an agonist of SAKT signaling (e.g. increasing SAKT activity and thus decreasing AKT activity). Conversely, e.g. in the remaining 40% of AMLs, where cancers exhibit decreased AKT activity, administration of an antagonist of SAKT signaling activity (e.g. decreasing SAKT activity and thus permiting increased AKT activity) can be therapeutic.

In one aspect, provided herein is a method of treating a subject having cancer, the method comprising determining, in a cancer cell sample obtained from the subject, the level of SAKT signaling activity, and administering a treatment to the subject, wherein a subject with a decreased level of SAKT signaling activity is administered a treatment comprising a therapeutically effective amount of an agonist of SAKT signaling, and wherein a subject with an increased level of SAKT signaling activity, as compared to a reference level, is administered a treatment comprising a therapeutically effective amount of an inhibitor of SAKT signaling. In one aspect, provided herein is a method of determining whether a cancer patient would benefit from treatment with an agonist of SAKT signaling, the method comprising: determining the level of SAKT signaling activity in a cancer cell sample obtained from the subject; wherein an agonist of SAKT signaling is indicated as an appropriate treatment if the level of SAKT signaling activity is decreased relative to a reference; and wherein an agonist of SAKT signaling is not indicated as an appropriate treatment if the level of SAKT signaling activity is not decreased relative to a reference. In one aspect, provided herein is a method of determining whether a cancer patient would benefit from treatment with an inhibitor of SAKT signaling, the method comprising: determining the level of SAKT signaling activity in a cancer cell sample obtained from the subject; wherein an inhibitor of SAKT signaling is indicated as an appropriate treatment if the level of SAKT signaling is increased relative to a reference; and wherein an inhibitor of SAKT signaling is not indicated as an appropriate treatment if the level of SAKT signaling activity is not increased relative to a reference.

As used herein, “SAKT signaling activity” refers to the activity of SAKT and/or the downstream effects of SAKT activity on the SAKT signaling pathway, as described elsewhere herein. In some embodiments, SAKT signaling activity can be the level of SAKT activity. In some embodiments, SAKT signaling activity can be the level of AKT, e.g. phosphorylated AKT and/or AKT phosphorylated at S473. In some embodiments, the level of SAKT signaling activity can be the level of RICTOR. In some embodiments, the level of SAKT signaling activity can be the level of FOXO phosphorylation, e.g. the level of FOXO3 phosphorylated at S253 and/or T32. Further non-limiting examples of markers of the level of SAKT signaling activity can include, the level of mTOR phosphorylated at S2448, the level of S6 phosphorylated at S235 and/or S236, the level of 4EBP1 phosphorylated at T37 and/or T46, the level of PRAS40 phosphorylated at T246, and the level of phosphorylated STAT3.

In some embodiments, increased SAKT activity can be determined by detecting one or more of the following: decreased levels of AKT phosphorylated at S473; decreased levels of RICTOR; decreased levels of FOXO phosphorylation, e.g. the level of FOXO3 phosphorylated at S253 and/or T32; decreased levels of mTOR phosphorylated at S2448; decreased levels of S6 phosphorylated at S235 and/or S236, decreased levels of 4EBP1 phosphorylated at T37 and/or T46; decreased levels of PRAS40 phosphorylated at T246, and/or decreased levels of phosphorylated STAT3. In some embodiments, decreased SAKT activity can be determined by detecting one or more of the following: increased levels of AKT phosphorylated at S473; increased levels of RICTOR; increased levels of FOXO phosphorylation, e.g. the level of FOXO3 phosphorylated at S253 and/or T32; increased levels of mTOR phosphorylated at S2448; increased levels of S6 phosphorylated at S235 and/or S236, increased levels of 4EBP1 phosphorylated at T37 and/or T46; increased levels of PRAS40 phosphorylated at T246, and/or increased levels of phosphorylated STAT3. In some embodiments, the level of SAKT activity can be determined by detecting a level of at least one marker of SAKT activity that indicates a modulation of SAKT activity. In some embodiments, the level of SAKT activity can be determined by detecting a level of a plurality of markers of SAKT activity that indicates a modulation of SAKT activity, e.g. two markers, three markers, four markers, up to and including all of the markers described in this paragraph.

In some embodiments, increased SAKT signaling activity can be determined by detecting a decreased level of AKT expression products and/or an increased level of FOXO expression product. In some embodiments, increased SAKT signaling activity can be determined by detecting a decreased level of AKT expression products and, optionally, an increased level of FOXO expression product. In some embodiments, decreased SAKT signaling activity can be determined by detecting an increased level of AKT expression products.

In some embodiments, AKT expression products can comprise phosphorylated AKT expression products. In some embodiments, AKT expression products can consist of phosphorylated AKT expression products. In some embodiments, the phosphorylated AKT expression product can be AKT phosphorylated at S473. Phosphorylation of S473 of AKT is accomplished by mTORC2 and is thus part of the SAKT signaling pathway. AKT can be phosphorylated at other residues, by polypeptides other than mTORC2 (e.g. at T308 by PDK1). In some embodiments, the level and/or changes in phosphorylation at S473 and other phosphorylated residues, e.g. T308 can be compared to determine if the SAKT signaling pathway is modulated and/or if an agent is specifically modulating the SAKT signaling pathway.

In some embodiments, SAKT signaling activity can be determined by determining the level of mTORC2 activity. By way of non-limiting example, decreased mTORC2 activity (e.g. as a result of increased SAKT signaling activity) can be determined by detecting a marker selected from the group consisting of: decreased levels of phosphorylated SGK; decreased levels of SGK phosphorylated at S422; decreased levels of phosphorylated PKCα; decreased levels of PKCα phosphorylated at S638; and decreased levels of AKT phosphorylated at S473. By way of further non-limiting example, increased mTORC2 activity (e.g. decreased SAKT signaling activity) can be determined by detecting a marker selected from the group consisting of: increased levels of phosphorylated SGK; increased level of SGK phosphorylated at S422; increased levels of phosphorylated PKCα; increased levels of PKCα phosphorylated at S638; and increased levels of AKT phosphorylated at S473.

In some embodiments, SAKT signaling activity can be determined by detecting the level of AKT activity. By way of non-limiting example, increased AKT activity (e.g. as a result of decreased SAKT acitivity) can be determined by detecting a marker selected from the group consisting of: decreased levels of GSK3β phosphorylated at S9; increased levels of FOXO, decreased levels of TSC2 phosphorylated at T1462 and S939; and decreased levels of mTORC1 phosphorylated at T246 of PRAS40. As a further non-limiting example, decreased AKT activity (e.g. increased SAKT acitivity) can be determined by detecting a marker selected from the group consisting of: increased levels of GSK3β phosphorylated at S9; decreased levels of FOXO, increased levels of TSC2 phosphorylated at T1462 and S939; and increased levels of mTORC1 phosphorylated at T246 of PRAS40.

As used herein, the term “AKT” refers to a serine-threonine protein kinase activated by a number of growth factors, including PDGF and which regulates a number of downstream targets, e.g. FOXO, TSC1, mTORC1, and GSK3b. It is the cellular homolog of the viral oncoprotein v-AKT, and is related to protein kinase-C (PKC) within the catalytic domain. However, AKT differs from the PKC family members by the presence of a pleckstrin homology (PH) domain at its N-terminus that is involved in the regulation of the activity of the enzyme by growth factors and intracellular signaling molecules. Various extracellular stimuli reportedly activate AKT through the phosphoinositide 3-kinase (PI 3-kinase) pathway. The lipid products of the PI 3-kinase reaction may activate AKT either by binding to the AKT pleckstrin homology domain (Franke, T. F. et al., 1997, Cell, 88:435:437), or by activating a protein kinase that phosphorylates AKT (Kohn, A. D., et al., J. Biol. Chem., 1996, 271:21920-21926; Stokoe et al., Science, 1997, 277:567-570). Activation of AKT reportedly inhibits apoptosis induced by growth factor withdrawal or irradiation in neural cells, fibroblasts, and lymphocytes (Franke, T. F., et al., Science, 1997, 275:665-668; Hemmings, Science, 1997, 275:628-630). It has been reported that AKT phosphorylates the pro-apoptotic protein Bad leading to Bad inactivation and cell survival (Datta, K., et al., Cell, 1997, 91:231-241; Peso, L., et al., Science, 1997, 278:687-689). AKT functions to promote tumorigenesis by phosphorylating and inactivating numerous substrates that antagonize cell growth and survival including PRAS40, GSK-3b, TSC2, BAD and the FOXO family transcription factors (Brunet et al., 1999; Cross et al., 1995; Datta et al., 1997; del Peso et al., 1997; Franke, 2008; Inoki et al., 2002; Kops and Burgering, 2000; Kops et al., 1999; Sancak et al., 2007; Tee et al., 2003; Wang et al., 2007). The kinase activity and substrate selectivity of AKT is controlled principally by two distinct phosphorylation events at threonine 308 (pAKT^{Thr308}) and serine 473 (pAKT^{Ser473}) via the actions of activated PI3K and mTORC2, respectively (Alessi et al., 1996; Alessi et al., 1997; Sarbassov et al., 2005; Stephens et al., 1998). Although pAKT^{Ser473} is dispensable for AKT-mediated phosphorylation of TSC2 and GSK-3b, pAKT^{Ser473} is required for phosphorylation and inactivation of the FOXOs (Guertin et al., 2006). The sequence of AKT for a number of species is well known in the art, e.g. human AKT (e.g. SEQ ID NO: 1, NCBI Ref Seq: NP_001014431 (polypeptide); SEQ ID NO: 2, NCBI Ref Seq: NM_005163 (mRNA); NCBI Gene ID: 207). Each of the references in the foregoing paragraph is incorporated by reference herein in its entirety.

As used herein, the term “mTORC2” refers to mTOR complex 2, a multi-protein complex comprising RICTOR, mTOR, GβL, and MAPKAP1, and which phosphorylates AKT at S473. The sequences of the components of mTORC2 are well known in the art, eg. human mTOR (mechanistic target of rapamycin) (e.g. SEQ ID NO: 5, NCBI Ref Seq: NP_004949 (polypeptide); SEQ ID NO: 6, NCBI Ref Seq: NM_004958 (mRNA); NCBI Gene ID: 2475), human GβL (e.g. NCBI Ref Seq: NP_001186102; NCBI Gene ID: 64223), and human MAPKAP1 (e.g. NCBI Ref Seq: NP_001006618; NCBI Gene ID: 79109). As used herein, the term “RICTOR” refers to a subunit of the mTORC2 complex which is negatively regulated by SAKT. The sequence of RICTOR for a number of species is well known in the art, e.g. human RICTOR (e.g. SEQ ID NO: 3, NCBI Ref Seq: NP_689969 (polypeptide); SEQ ID NO: 4 NCBI Ref Seq: NM_152756 (mRNA); NCBI Gene ID: 253260).

As used herein, “FOXO” or “Forkhead Box O” refers to a family of transcription factors, comprised of four highly related members; FOXO1, FOXO3, FOXO4 and FOXO6, which are direct downstream targets of AKT (Arden, 2006; Brunet et al., 1999; Burgering, 2008; Fu and Tindall, 2008; Kops and Burgering, 2000). While not wishing to be bound by theory, it has been postulated that in the absence of active AKT, FOXOs localize to the nucleus where they regulate the transcription of genes involved in cell cycle arrest, apoptosis and reactive oxygen species (ROS) detoxification. Upon AKT-mediated phosphorylation, FOXOs are exported to the cytoplasm and undergo proteasome-mediated degradation (Brunet et al., 1999; Carter and Brunet, 2007). The sequence of FOXO polypeptides for a number of species is well known in the art, e.g. human FOXO1 (e.g. SEQ ID NO: 7, NCBI Ref Seq: NP_002006 (polypeptide); SEQ ID NO: 8 NCBI Ref Seq: NM_002015 (mRNA); NCBI Gene ID: 2308); human FOXO3 (e.g. SEQ ID NO: 9, NCBI Ref Seq: NP_001446 (polypeptide); SEQ ID NO: 10 NCBI Ref Seq: NM_001455 (mRNA); NCBI Gene ID: 2309); human FOXO4 (e.g. SEQ ID NO: 11, NCBI Ref Seq: NP_005929 (polypeptide); SEQ ID NO: 12 NCBI Ref Seq: NM_005938 (mRNA); NCBI Gene ID: 4303); human FOXO6 (e.g. SEQ ID NO: 13, NCBI Ref Seq: XP_002342143 (polypeptide); SEQ ID NO: 14 NCBI Ref Seq: XM_002342102 (mRNA); NCBI Gene ID: 100132074). In some embodiments, “FOXO” can refer to one or more of FOXO1, FOXO3, FOXO4, and/or FOXO6, e.g. 1 FOXO gene, two FOXO genes, three FOXO genes, or all four FOXO genes.

In some embodiments, the level of a marker of SAKT signaling activity is the expression level of that marker, e.g. the level of mRNA transcript expression product of that gene and/or the level of polypeptide expression product of that gene. In some embodiments, determining and/or detecting the level of a marker of SAKT signaling activity can comprise contacting the sample with a probe specific for the marker, e.g. a primer specific for the mRNA expression product of the marker and/or with an antibody reagent specific for the polypeptide expression product of the marker. A probe, or detection agent can be any agent which can specifically detect the presence of the target (e.g. bind specifically to the target) according to an assay described herein, e.g. a probe can be a nucleic acid probe or primer specific for the target or an agent which specifically binds to a target polypeptide. In some embodiments, the probe can comprise a detectable signal or be capable of generating a detectable signal. In some embodiments, the probe can be an antibody reagent. In some embodiments, the probe can be a monoclonal antibody and/or comprise CDRs of a monoclonal antibody.

In some embodiments, the methods and assays described herein include (a) transforming gene expression products of a marker of SAKT signaling activity into detectable gene targets; (b) measuring the amount of the detectable gene targets; and (c) comparing the amount of each detectable gene target to an amount of a reference to determine if the amount of the detectable gene target is statistically significantly different than that of the amount of the reference level. As used herein, the term “transforming” or “transformation” refers to changing an object or a substance, e.g., biological sample, nucleic acid or protein, into another substance. The transformation can be physical, biological or chemical. Exemplary physical transformation includes, but is not limited to, pre-treatment of a biological sample. A biological/chemical transformation can involve at least one enzyme and/or a chemical reagent in a reaction. For example, a DNA sample can undergo enzymatic replication, e.g., by polymerase chain reaction (PCR). In some embodiments, determining and/or detecting the level of SAKT signaling activity can comprise transforming the expression product of the marker into a detectable molecule, e.g. a detectable RT-PCR product or detectable marker polypeptide-antibody reagent complex.

In some embodiments, the level of a marker of SAKT signaling activity can be the level of the polypeptide expression product of the marker. Assays for detecting polypeptides are well known in the art and include, but are not limited to, ELISA (enzyme linked immunosorbent assay), western blot, immunoprecipitation, immunohistochemistry, and immunofluorescence using detection reagents such as an antibody, antibody reagent, or protein binding agent. Alternatively, a peptide can be detected in a subject by introducing into the subject a labeled anti-peptide antibody and other types of detection agent. For example, the antibody can be labeled with a radioactive marker whose presence and location in the subject is detected by standard imaging techniques.

Antibodies specific for, e.g. AKT and FOXO (e.g. FOXO1, FOXO3, FOXO4, and/or FOXO6) are commercially available, (e.g. Cat. Nos. ab8805; ab126826; ab53287, ab128908, and ab48730 respectively; Abcam; Cambrige, MA) and can be used for the purposes of the methods and assays described herein to measure polypeptide expression levels. In some emboidments, the antibody can be specific for a particular phosphorylated form of a marker described herein, e.g. antibodies specific for AKT phosphorylated at S473 are commercially available (e.g. Cat. No. ab66138; Abcam; Cambrige, Mass. Ab 66138). Alternatively, since the amino acid sequences for the markers of SAKT signaling activity have been assigned NCBI accession numbers for different species such as human, mouse and rat (e.g. the human sequence of AKT is provided as SEQ ID NO: 2) and are publically available at NCBI website, one of skill in the art can raise their own antibodies against these proteins of interest for the methods and assays described herein. In some embodiments, the antibody reagent is detectably labeled or capable of generating a detectable signal.

In some embodiments, immunohistochemistry (“IHC”) and immunocytochemistry (“ICC”) techniques can be used. IHC is the application of immunochemistry to tissue sections, whereas ICC is the application of immunochemistry to cells or tissue imprints after they have undergone specific cytological preparations such as, for example, liquid-based preparations. Immunochemistry is a family of techniques based on the use of an antibody, wherein the antibodies are used to specifically target molecules inside or on the surface of cells. The antibody typically contains a marker that will undergo a biochemical reaction, and thereby experience, e.g. a change in color, upon encountering the targeted molecules or upon treatment with a chemical agent. In some instances, signal amplification can be integrated into the particular protocol, wherein a secondary antibody, that includes the marker signal or marker activity (e.g. an enzyme activity), follows the application of a specific antibody.

In some embodiments, determining the level of SAKT signaling activity can comprise; (a) contacting a biofluid test sample obtained from a subject with a detectable anti-AKT antibody reagent and optionally, an anti-FOXO antibody reagent; and (b) detecting the presence or intensity of a detectable signal; wherein the expression level of AKT polypeptide, and optionally, FOXO polypeptide, is indicated by the level of the detectable signal.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments (see, e.g. de Wildt et al., Eur J. Immunol. 1996; 26(3):629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The terms “antigen-binding fragment” or “antigen-binding domain”, which are used interchangeably herein refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., U.S. Pat. Nos. 5,260,203, 4,946,778, and 4,881,175; Bird et al. (1988) Science 242:423-426; and Huston et al. (1988) Proc. Natl. Acad. Sci. USA 85:5879-5883. Antibody fragments can be obtained using any appropriate technique including conventional techniques known to those of skill in the art. The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition, irrespective of how the antibody was generated.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. Accordingly, as used herein, “selectively binds” or “specifically binds” refers to the ability of an antibody reagent (e.g., an antibody or portion thereof) to bind to a target, such as a marker of SAKT signaling activity as described herein, with a KD 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less.

The term “label” refers to a composition capable of producing a detectable signal indicative of the presence of an antibody reagent (e.g. a bound antibody reagent). Suitable labels include radioisotopes, nucleotide chromophores, enzymes, substrates, fluorescent molecules, chemiluminescent moieties, magnetic particles, bioluminescent moieties, and the like. As such, a label is any composition detectable by spectroscopic, photochemical, biochemical, immunochemical, electrical, optical or chemical means.

In some embodiments, the expression level of a marker of SAKT signaling activity can be the level of mRNA transcript expression product of the marker. Such molecules can be isolated, derived, or amplified from a biological sample, such as a biopsy or blood sample. Assays for detecting mRNA transcripts are well known in the art and include, but are not limited to, PCR procedures, RT-PCR, Northern blot analysis, RNAse protection assay, microarray analysis, hybridization methods etc. In some embodiments, mRNA transcript expression product levels are assayed using reverse transcription polymerase chain reaction (RT-PCR).

The nucleic acid sequences of, e.g. SAKT and FOXO have been assigned NCBI accession numbers for different species such as human, mouse and rat. In particular, the NCBI accession numbers for the nucleic acid sequences of the human SAKT and FOXO expression products are included herein (SEQ ID NOs: 8, 10, 12, and 14). Accordingly, a skilled artisan can design appropriate primers based on the known sequence for determining the mRNA level of the respective gene. In some embodiments, the level of AKT or FOXO expression products can be determined by measuring the level of RNA transcripts. In some embodiments, the RNA transcript level can be measured using reverse transcription polymerase chain reaction (RT-PCR).

Nucleic acid and ribonucleic acid (RNA) molecules can be isolated from a particular biological sample using any of a number of procedures, which are well-known in the art, the particular isolation procedure chosen being appropriate for the particular biological sample. For example, freeze-thaw and alkaline lysis procedures can be useful for obtaining nucleic acid molecules from solid materials; and proteinase K extraction can be used to obtain nucleic acid from blood (Roiff, A et al. PCR: Clinical Diagnostics and Research, Springer (1994)).

In general, the PCR procedure describes a method of gene amplification which is comprised of (i) sequence-specific hybridization of primers to specific genes or sequences within a nucleic acid sample or library, (ii) subsequent amplification involving multiple rounds of annealing, elongation, and denaturation using a thermostable DNA polymerase, and (iii) screening the PCR products for a band of the correct size. The primers used are oligonucleotides of sufficient length and appropriate sequence to provide initiation of polymerization, i.e. each primer is specifically designed to be complementary to a strand of the genomic locus to be amplified. In an alternative embodiment, mRNA level of gene expression products described herein can be determined by reverse-transcription (RT) PCR and by quantitative RT-PCR (QRT-PCR) or real-time PCR methods. Methods of RT-PCR and QRT-PCR are well known in the art.

In some embodiments, multiple markers of SAKT signaling activity can be determined as described herein, e.g. the level of AKT phosphorylated at S473 and the level of FOXO polypeptide. In some embodiments, multiple techniques can be used to detect markers of SAKT signaling activity, e.g. the level of a polypeptide marker can be determined using immunochemistry and the level of an mRNA transcript of a second marker can be determined using RT-PCR.

As described elsewhere herein, SAKT activity specifically affects RICTOR and thus phosphorylation at S473 of AKT, and therefore specifically modulates the effect of AKT on FOXO, as opposed to, e.g. GSK3b and TSC2. Accordingly, in some embodiments, the modulation of SAKT signaling activity can be confirmed to be SAKT signaling activity, rather than general modulation of AKT activity by determining the level of markers of RICTOR signaling. By way of non-limiting example, modulated mTORC2 activity can be determined by detecting a marker selected from the group consisting of SGK phosphorylated at S422; PKCα phosphorylated at S638; and AKT phosphorylated at S473 as described elsewhere herein. In some embodiments, the levels of all of SGK phosphorylated at S422; PKCα phosphorylated at S638; and AKT phosphorylated at S473 can be determined. In some embodiments, the levels of, e.g. markers of mTORC1 (which is not regulated by SAKT) activity can also be determined in order to confirm that SAKT signaling pathway activity, and not general AKT activity is modulated. Non-limiting examples of markers of mTORC1 activity can include S6k phosphorylated at T389 and increased transcription of Hif1.

As described elsewhere herein, SAKT activity specifically modulates mTORC2 activity but not mTORC1 activity. Accordingly, in some embodiments, the specific modulation of SAKT signaling activity can be confirmed by detecting a change in mTORC2 activity and no detectable change in mTORC 1 activity. If a change in mTORC 1 activity is detected, it can indicate that SAKT signaling activity has not been modulated, or at least not exclusively modulated.

In some embodiments, modulation of SAKT signaling activity can be confirmed to be SAKT signaling activity, rather than general modulation of AKT pathway activity by determining the level of markers of AKT signaling. By way of non-limiting example, modulated AKT activity can be determined by detecting a marker selected from the group consisting of increased levels of FOXO. In some embodiments, the levels of, e.g. the levels of markers of AKT signaling axes which do not require phosphorylation at S473 can also be determined in order to confirm that SAKT signaling pathway activity, and not general AKT activity is modulated. Non-limiting examples of markers of AKT signaling axes which do not require phosphorylation at S473 can include the level of GSK3b phosphorylated at S9 and/or the level of TSC2 phosphorylated at T1462 and/or S939.

In one aspect, described herein is an assay comprising: (a) contacting a cancer cell sample obtained from a subject with a detectable anti-AKT antibody reagent; and (b) detecting the presence or intensity of a detectable signal; wherein an increase in the level of AKT polypeptide, indicated by the level of the detectable signal, relative to a reference level indicates the subject is in need of treatment with an agonist of SAKT signaling activity; and wherein a decrease in the level of AKT polypeptide, indicated by the level of the detectable signal, relative to a reference level indicates the subject is in need of treatment with an inhibitor of SAKT signaling activity. In some embodiments, the assay can further comprise contacting the cancer cell sample with a detectable anti-Foxo antibody reagent. In some embodiments, the anti-AKT antibody reagent can be specific for AKT polyeptide phosphorylated at S473.

For cancer in which SAKT signaling activity is not perturbed, the assay would indicate no treatment pertaining to SAKT activity should be administered.

A reference level can be the level in a sample obtained from a subject not having, or diagnosed as having, cancer. In some embodiments, a reference level can be the level in a sample previously obtained from the subject. In some embodiments, a reference level can be the average level in a population of subjects not having, or diagnosed as having, cancer who have a similar age, sex, ethnicity, genetic background, and health/lifestyle as the subject.

In some embodiments, a level of SAKT signaling is increased relative to the reference if the level is increased by at least 25%, e.g. at least 25%, at least 50%, at least 100%, at least 200% , at least 300%, at least 500% or more relative to the reference level. In some embodiments, a level of SAKT signaling is increased relative to the reference if the level of a marker of SAKT signaling differs from the reference level by a statistically significant amount as described elsewhere herein. In some embodiments, a level of SAKT signaling is increased relative to the reference if the level of a marker of SAKT signaling differs from the reference level by at least 25% lower, at least 50% lower, at least 75% lower, at least 90% lower, or lower as described elsewhere herein.

In some embodiments, a level of SAKT signaling is decreased relative to the reference if the level is increased by at least 25%, e.g. at least 25%, at least 50%, at least 70%, at least 80%, at least 90%, at least 95% or more than the reference level. In some embodiments, a level of SAKT signaling is decreased relative to the reference if the level of a marker of SAKT signaling differs from the reference level by a statistically significant amount as described elsewhere herein. In some embodiments, a level of SAKT signaling is decreased relative to the reference if the level of a marker of SAKT signaling differs from the reference level by at least 25%, at least 50%, at least 75%, at least 90% or more as described elsewhere herein.

In some embodiments, an increased level can be a level at least 25% greater than a reference level, e.g. at least 25%, at least 50%, at least 100%, at least 200%, at least 300%, or at least 500% or more than a reference level.

In some embodiments, a decreased level can be a level at least 25% less than a reference level, e.g. at least 25%, at least 50%, at least 70%, at least 80%, at least 90%, at least 95%, at least 98% or less of the reference level.

In some embodiments, the expression of no more than 20 other genes can be determined. In some embodiments, the expression of no more than 10 other genes can be determined.

In some embodiments, the level of a marker of SAKT signaling activity can be normalized relative to the expression level of one or more reference genes or reference proteins.

In some embodiments, the methods, assays, and systems described herein relate to determining the level of SAKT signaling activity in a cancer cell, e.g. a cancer cell sample obtained from a subject. In some embodiments, the cancer cell can be a hematopoietic cancer cell. In some embodiments, the cancer cell can be an epithelial cancer cell. Non-limiting examples of epithelial cancers can include carcinoma; adenocarcinoma; basal cell carcinoma; squamous cell carcinoma; large cell carcinoma; small cell carcinoma; colorectal adenocarcinoma; lung cancer; breast cancer; prostate cancer; colon cancer; rectal cancer; pancreatic cancer; kidney cancer; ovarian cancer; stomach cancer; intestinal cancer; oral cancer; esophageal cancer; lip cancer; bladder cancer; cervical cancer; skin cancer; hepatocellular carcinoma; and renal cell carcinoma. In some embodiments, the sample can comprise tumor and/or diseased cells from a subject diagnosed with and/or in need of treatment for a hematopoietic or epithelial cancer.

The term “sample” or “test sample” as used herein denotes a sample taken or isolated from a biological organism, e.g., a tumor sample from a subject. Exemplary biological samples include, but are not limited to, a biofluid sample; serum; plasma; blood or a product thereof, serum, plasma, and/or a tumor biopsy; a tumor sample; and/or tissue sample etc. The term also includes a mixture of the above-mentioned samples. The term “test sample” also includes untreated or pretreated (or pre-processed) biological samples. In some embodiments, a test sample can comprise cells from subject. In some embodiments, a test sample can be a tumor cell test sample, e.g. the sample can comprise cancerous cells, cells from a tumor, and/or a tumor biopsy, or in the case of a hematopietic cancer, blood cells of one or more types. In some embodiments, the sample can comprise blood or a product thereof, serum, plasma, and/or a tumor biopsy.

The test sample can be obtained by removing a sample of cells from a subject, but can also be accomplished by using previously isolated cells (e.g. isolated at a prior timepoint and isolated by the same or another person). In addition, the test sample can be freshly collected or a previously collected sample.

In some embodiments, the test sample can be an untreated test sample. As used herein, the phrase “untreated test sample” refers to a test sample that has not had any prior sample pre-treatment except for dilution and/or suspension in a solution. Exemplary methods for treating a test sample include, but are not limited to, centrifugation, filtration, sonication, homogenization, heating, freezing and thawing, and combinations thereof. In some embodiments, the test sample can be a frozen test sample, e.g., a frozen tissue. The frozen sample can be thawed before employing methods, assays and systems described herein. After thawing, a frozen sample can be centrifuged before being subjected to methods, assays and systems described herein. In some embodiments, the test sample is a clarified test sample, for example, by centrifugation and collection of a supernatant comprising the clarified test sample. In some embodiments, a test sample can be a pre-processed test sample, for example, supernatant or filtrate resulting from a treatment selected from the group consisting of centrifugation, filtration, thawing, purification, and any combinations thereof. In some embodiments, the test sample can be treated with a chemical and/or biological reagent, for example, to protect and/or maintain the stability of the sample, including biomolecules (e.g., nucleic acid and protein) therein, during processing. One exemplary reagent is a protease inhibitor, which is generally used to protect or maintain the stability of protein during processing. The skilled artisan is well aware of methods and processes appropriate for pre-processing of biological samples required for determination of SAKT signaling activity as described herein.

In some embodiments, the methods, assays, and systems described herein can further comprise a step of obtaining a test sample from a subject.

In some embodiments, described herein is the administration of (or contacting a cell with) a modulator (e.g. an agonist or inhibitor) of the SAKT signaling pathway. In some embodiments, a modulator of the SAKT signaling pathway can be specific, e.g. it does not modulate the activity of other AKT signaling axes, e.g. AKT-mTORC1 signaling, and/or AKT-GSK3b signaling. In some embodiments, a modulator of SAKT signaling activity can be an agent that affects only the SAKT signaling pathway, i.e. it modulates the activity and/or level of a marker of the SAKT signaling pathway specifically and/or exclusively with respect to the activity and/or level of a marker of AKT signaling outside of the SAKT signaling axis.

An agonist of SAKT signaling is an agent that increases SAKT signaling activity. As used herein, the term “agonist” refers to an agent which increases the expression and/or activity of the target by at least 10% or more, e.g. by 10% or more, 50% or more, 100% or more, 200% or more, 500% or more, or 1000% or more. In some embodiments, an agonist of SAKT signaling can be an agonist of SAKT. In some embodiments, an agonist of SAKT can be an antibody reagent agonist, e.g. an antibody reagent that binds SAKT in a manner that mimics a natural ligand and/or induces an activating conformational change. In some embodiments, an agonist of SAKT can be a nucleic acid encoding SAKT such that expression from the nucleic acid increases the level of SAKT. In some embodiments, an agonist of SAKT signaling can be an inhibitor of RICTOR. In some embodiments, an agonist of SAKT signaling can be an inhibitor of mTORC2. In some embodiments, an agonist of SAKT signaling can be an inhibitor of the interaction between RICTOR and mTOR.

Inhibitors of mTORC2 are known in the art and include, by way of non-limiting example, TORIN1, AZD8055, INK128, and Palomid-529. Further examples of mTORC2 inhibitors include OSI-027; MK8669; TOP216; TORISEL; CERTICAN; ABI-009; KU-0063794; AZD2014; NVP-BGT226; PF-04691502; PP242; XL765; EXEL-2044; EXEL-3885; EXEL-4431; EXEL-7518 and those described, e.g. in US Patent Publication 2012/0165334; 2011/0224223; 2012/0114739; 2010/0184760; 2012/0178715; Bhagwat and Crew. Curr Opin Investig Drugs 2010 11:638-645; which are incorporated by reference herein in their entireties. In some embodiments, an inhibitor of mTORC2 can be specific for inhibition of mTORC2, e.g. it is not an inhibitor of mTORC1.

Inhibitors of RICTOR are known in the art and include, by way of non-limiting example, NVP-BEZ235.

An inhibitor of SAKT signaling is an agent that decreases SAKT signaling activity. As used herein, the term “inhibitor” refers to an agent which decreases the expression and/or activity of the target by at least 10% or more, e.g. by 10% or more, 20% or more, 50% or more, 70% or more, 80% or more, 90% or more, 95% or more, or 98% or more. In some embodiments, an inhibitor of SAKT signaling can be an inhibitor of SAKT. In some embodiments, an inhibitor of SAKT can be an inhibitory nucleic acid molecule. In some embodiments, an inhibitor of SAKT can be an antibody reagent. In some embodiments, an inhibitor of SAKT signaling can be an agonist of RICTOR. In some embodiments, an inhibitor of SAKT signaling can be an agonist of mTORC2.

In some embodiments of any of the aspects described herein, the subject can be a human subject.

In one aspect, described herein is a method of suppressing AKT activity, the method comprising administering an agonist of SAKT, e.g. an agonist of SAKT activity and/or expression.

As discussed elsewhere herein, the SAKT signaling pathway, in at least some contexts, has been demonstrated to modulate cell survival but not cell proliferation. Accordingly, modulation of the SAKT signaling pathway can promote cell survival (e.g. of injured cells) without promoting cell proliferation and the side effects associated with excess proliferation. In one aspect, described herein is a method of treating ischemic injury, the method comprising administering an agonist of SAKT signaling.

As described elsewhere herein, the inventors have discovered a novel regulator of AKT signaling, which is known to control responses to a number of growth factors. Accordingly, provided herein is a method of altering the sensitivity of a cell to a growth factor, the method comprising; contacting the cell with an inhibitor of SAKT signaling activity to render the cell more sensitive to the growth factor; or contacting the cell with an agonist of SAKT signaling activity to render the cell less sensitive to the growth factor. By way of non-limiting examples, contacting a cell with an agonist of SAKT to render the cell less sensitive to a growth factor can slow the growth of a cancer cell and/or decrease unwanted angiogenesis, while contacting a cell with an inhibitor of SAKT signaling activity to render the cell more senstivie to the growth factor can increase the rate of wound healing or decrease insulin resistance. In some embodiments, the growth factor can be selected from the group consisting of insulin; epidermal growth factor (EGF); hematopoietic cytokine; hematopoietic growth factor; vascular endothelial growth factor (VEGF); insulin-like growth factor-1 (IGF); platelet-derived growth factor (PDGF), granulocyte colony-stimulating factor (G-CSF); platelet activating factor, and macrophage stimulating factor. In some embodiments, the growth factor can be selected from the group consisting of insulin and EGF.

In some embodiments, the agonist or inhibitor of SAKT signaling activity can be administered to a subject. In some embodiments, the subject can be in need of treatment for a condition selected from the group consisting of: insulin resistance; diabetes; cancer; proliferative diseases; and wound healing. In some embodiments, the agonist or inhibitor can be an antibody reagent or a small molecule. In some embodiments, the agonist or inhibitor can be a nucleic acid, e.g. an inhibitory nucleic acid or a nucleic acid encoding, e.g. SAKT. Nucleic acid agents are particularly effective when it is desired to modulate the response of a cell in the liver of a subject, e.g. in the case of insulin resistance or hepatocellular cancer, as systemically administered nucleic acids are known to be effectively taken up by the liver.

As described herein, the methods described herein can modulate the level and/or activity of STAT3 (e.g. the level of phosphorylation of STAT3). STAT3 activity can promote the differentiation and/or proliferation of cells in the hematopoietic lineage, e.g. hematopoietic stem and progenitor cells (see, e.g. Zhang et al. Blood 2010 116:2462-71 and Hankey. Front Biosci 2009 14:5273-5290; each of which is incorporated by reference herein in its entirety). Accordingly, the methods described herein can relate to methods for promoting or inhibiting hematopoiesis. By way of non-limiting example, administration of an agonist of SAKT signaling activity can decrease hematopoietic reconstitution capacity, whereas administration of an inhibitor of SAKT signaling activity can increase hematopoietic reconstitution capacity.

In some aspects, the invention described herein is directed to systems (and computer readable media for causing computer systems) for obtaining data from at least one sample obtained from at least one subject, the system comprising 1) a measuring module configured to measure the level of SAKT signaling activity, in a test sample obtained from a subject, 2) a storage module configured to store output data from the measuring module, 3) a comparison module adapted to compare the data stored on the storage module with a reference level, and to provide a retrieved content, and 4) a display module for displaying whether the sample comprises a level of SAKT signaling activity which is significantly increased or decreased relative to the reference level and/or displaying the relative SAKT signaling activity.

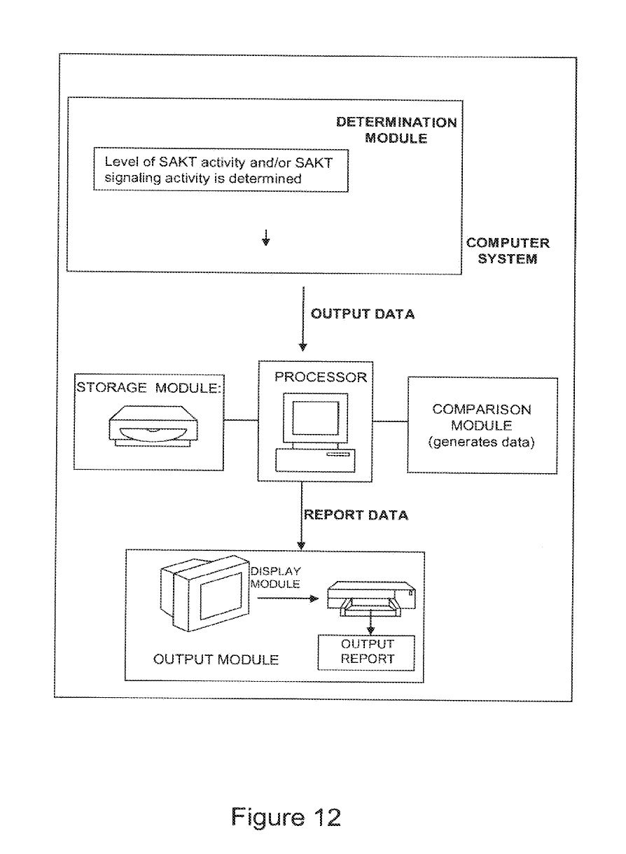

In one embodiment, described herein is a computer system for determining the appropriate treatment for a subject having cancer, the system comprising: a measuring module configured to measure the level of SAKT signaling activity, in a test sample obtained from a subject; a storage module configured to store output data from the determination module; a comparison module adapted to compare the data stored on the storage module with a reference level, and to provide a retrieved content, and a display module for displaying whether the sample comprises a level of SAKT signaling activity which is significantly increased or decreased relative to the reference level and/or displaying the relative SAKT signaling activity and (b) at least one processor for executing the computer program (see FIG. 12).

In some embodiments, the measuring module can measure the presence and/or intensity of a detectable signal from an immunoassay indicating the level of a marker of SAKT signaling activity in the test sample. Exemplary embodiments of a measuring module can include an automated RT-PCR or automated immunoassay, etc.

The measuring module can comprise any system for detecting a signal elicited from an assay to determine the level of SAKT signaling activity as described above herein. In some embodiments, such systems can include an instrument, e.g., AU2700 (Beckman Coulter; Brea, Calif.) for quantitative measurement of polypeptides or a qRT-PCR instrument (e.g. CFX96 TOUCH™ Real-Time PCR Detection System). In another embodiment, the measuring module can comprise multiple units for different functions, such as measurement of phosphorylated AKT (and/or detectable signals from AKT-specific antibody reagents) and measurement of the level of FOXO (and/or detectable signals from FOXO-specific antibody reagents or an RT-PCR assay specific for FOXO transcripts). In one embodiment, the measuring module can be configured to perform the methods described elsewhere herein, e.g. an immunoassay or RT-PCR, or detection of any detectable label or signal.

In some embodiments, the measuring system or a further module can be configured to process samples, e.g. to isolate polypeptides and/or nucleic acids from sample comprising cells for use in the assays described herein.

The term “computer” can refer to any non-human apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a micro-computer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

The term “computer-readable medium” can refer to any storage device used for storing data accessible by a computer, as well as any other means for providing access to data by a computer. Examples of a storage-device-type computer-readable medium include: a magnetic hard disk; a floppy disk; an optical disk, such as a CD-ROM and a DVD; a magnetic tape; a memory chip. The term a “computer system” can refer to a system having a computer, where the computer comprises a computer-readable medium embodying software to operate the computer. The term “software” is used interchangeably herein with “program” and refers to prescribed rules to operate a computer. Examples of software include: software; code segments; instructions; computer programs; and programmed logic.

The computer readable storage media can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (erasable programmable read only memory), EEPROM (electrically erasable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing. “Computer-readable storage medium” as the term is used herein does not include a signal or a carrier wave.

Computer-readable data embodied on one or more computer-readable media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable media on which such instructions are embodied may reside on one or more of the components of either of a system, or a computer readable storage medium described herein, may be distributed across one or more of such components.

The computer-readable media may be transportable such that the instructions stored thereon can be loaded onto any computer resource to implement the aspects of the present invention discussed herein. In addition, it should be appreciated that the instructions stored on the computer-readable medium, described above, are not limited to instructions embodied as part of an application program running on a host computer. Rather, the instructions may be embodied as any type of computer code (e.g., software or microcode) that can be employed to program a computer to implement aspects of the present invention. The computer executable instructions may be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

Embodiments of the invention can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules can perform other functions, thus the modules are not limited to having any particular functions or set of functions.

The functional modules of certain embodiments of the invention include at minimum a measuring module, a storage module, a computing module, and a display module. The functional modules can be executed on one, or multiple, computers, or by using one, or multiple, computer networks. The measuring module has computer executable instructions to provide e.g., levels of a marker of SAKT signaling activity in computer readable form.

The information determined in the measuring system can be read by the storage module. As used herein the “storage module” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage modules also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage module is adapted or configured for having recorded thereon, for example, sample name, biomolecule assayed and the level of said biomolecule. Such information may be provided in digital form that can be transmitted and read electronically, e.g., via the Internet, on diskette, via USB (universal serial bus) or via any other suitable mode of communication.

As used herein, “stored” refers to a process for encoding information on the storage module. Those skilled in the art can readily adopt any of the presently known methods for recording information on known media to generate manufactures comprising expression level information.

In some embodiments of any of the systems described herein, the storage module stores the output data from the measuring module. In additional embodiments, the storage module stores reference information such as levels of SAKT signaling activity and/or markers thereof in healthy subjects, and/or subjects not having a cancer, and/or a prior sample obtained from the subject.

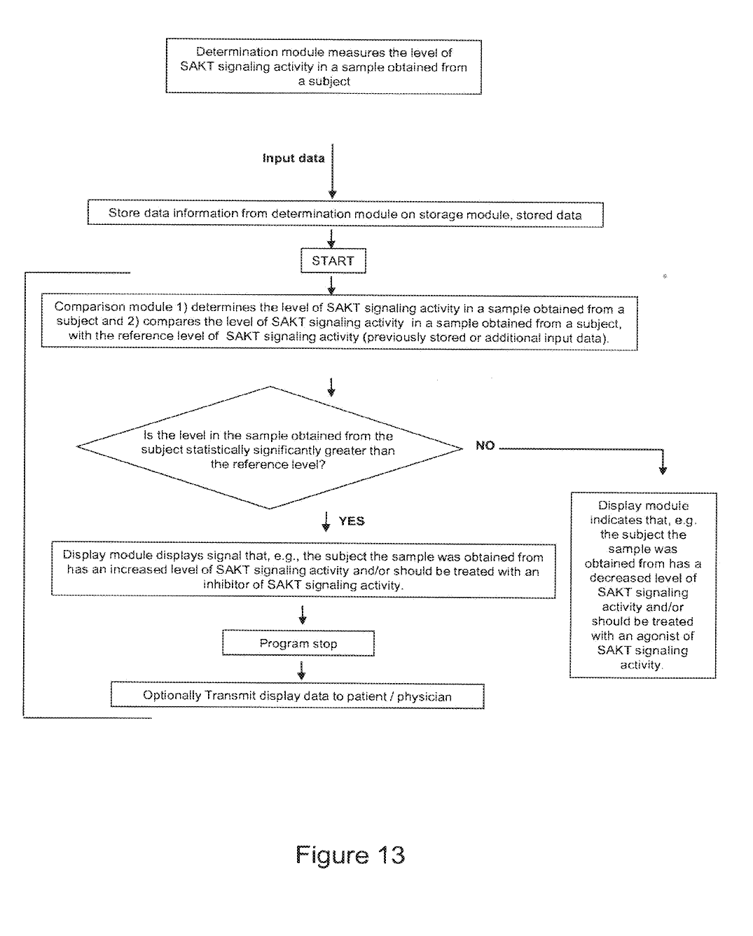

The “computing module” can use a variety of available software programs and formats for computing the level of SAKT signaling activity. Such algorithms are well established in the art. A skilled artisan is readily able to determine the appropriate algorithms based on the size and quality of the sample and type of data. The data analysis tools and equations described herein can be implemented in the computing module of the invention. In some embodiments, the computing module can comprise a computer and/or a computer system. In one embodiment, the computing module further comprises a comparison module, which compares the level of SAKT signaling activity (or marker thereof) in a sample obtained from a subject as described herein with a reference level as described herein (see, e.g. FIG. 13). By way of an example, when the level of SAKT signaling activity in a sample obtained from a subject is measured, a comparison module can compare or match the output data with the mean level of SAKT signaling activity in a population of subjects not having signs or symptoms of cancer (i.e. a reference level). In certain embodiments, the mean level of SAKT signaling activity in a population of subjects not having signs or symptoms of cancer can be pre-stored in the storage module. During the comparison or matching process, the comparison module can determine whether the level of SAKT signaling activity in a sample obtained from a subject is statistically significantly different than the reference level. In various embodiments, the comparison module can be configured using existing commercially-available or freely-available software for comparison purpose, and may be optimized for particular data comparisons that are conducted.

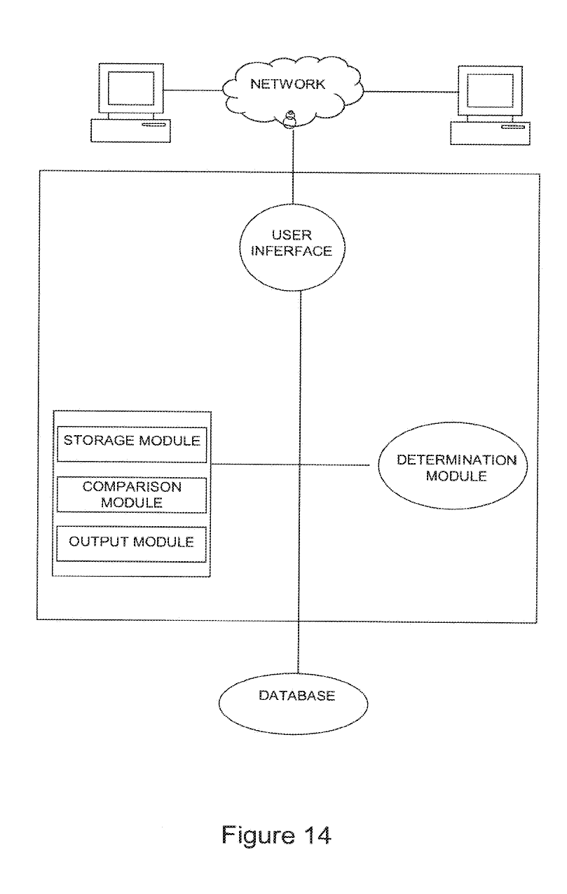

The computing and/or comparison module, or any other module of the invention, can include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware—as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). In some embodiments users can directly access data (via Hypertext links for example) residing on Internet databases using a HTML interface provided by Web browsers and Web servers (FIG. 14).

The computing and/or comparison module provides a computer readable comparison result that can be processed in computer readable form by predefined criteria, or criteria defined by a user, to provide content based in part on the comparison result that may be stored and output as requested by a user using an output module, e.g., a display module.

In some embodiments, the content displayed on the display module can be a report, e.g. the level of SAKT signaling activity and/or the level of a marker of SAKT signaling activity in the sample obtained from a subject. In some embodiments, the report can denote raw values of the SAKT signaling activity or marker in the test sample or it indicates a percentage or fold change in SAKT signaling activity (or marker thereof) as compared to a reference level, and/or provides a signal that the subject is in need of treatement with an agonist or inhibitor of SAKT signaling.

In some embodiments if the computing module determines that the level of SAKT signaling activity in the test sample obtained from a subject is less by a statistically significant amount than the reference level, the display module displays a signal indicating that the expression levels in the sample obtained from a subject are less than those of the reference level. In some embodiments, if the computing module determines that the level of SAKT signaling activity in the test sample obtained from a subject is less by a statistically significant amount than the reference level, the display module displays a signal indicating that the appropriate treatment for the subject is an agonist of SAKT signaling. In some embodiments, if the computing module determines that the level of SAKT signaling activity in the test sample obtained from a subject is greater by a statistically significant amount than the reference level, the display module displays a signal indicating that the expression levels in the sample obtained from a subject are greater than those of the reference level. In some embodiments, if the computing module determines that the level of SAKT signaling activity in the test sample obtained from a subject is greater by a statistically significant amount than the reference level, the display module displays a signal indicating that the appropriate treatment for the subject is an inhibitor of SAKT signaling. In some embodiments, the signal indicates the degree to which the level of SAKT signaling activity in the sample obtained from a subject varies from the reference level.

In one embodiment of the invention, the content based on the computing and/or comparison result is displayed on a computer monitor. In one embodiment of the invention, the content based on the computing and/or comparison result is displayed through printable media. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, California, or any other type of processor, visual display devices such as flat panel displays, cathode ray tubes and the like, as well as computer printers of various types.

In one embodiment, a World Wide Web browser is used for providing a user interface for display of the content based on the computing/comparison result. It should be understood that other modules of the invention can be adapted to have a web browser interface. Through the Web browser, a user can construct requests for retrieving data from the computing/comparison module. Thus, the user will typically point and click to user interface elements such as buttons, pull down menus, scroll bars and the like conventionally employed in graphical user interfaces.

Systems and computer readable media described herein are merely illustrative embodiments of the invention for determining the level of SAKT signaling activity in a sample obtained from a subject, and therefore are not intended to limit the scope of the invention. Variations of the systems and computer readable media described herein are possible and are intended to fall within the scope of the invention. The modules of the machine, or those used in the computer readable medium, may assume numerous configurations. For example, function may be provided on a single machine or distributed over multiple machines.

In some embodiments, the methods described herein relate to treating a subject having or diagnosed as having, e.g. cancer, diabetes, or ischemic injury. Subjects having, e.g. cancer can be identified by a physician using current methods of diagnosing cancer. Symptoms and/or complications of cancer which characterize these conditions and aid in diagnosis are well known in the art and include but are not limited to, growth of a tumor, impaired function of the organ or tissue harboring cancer cells, etc. Tests that may aid in a diagnosis of, e.g. cancer include, but are not limited to, tissue biopsies and histological examination. A family history of cancer or exposure to risk factors for cancer (e.g. smoking or radiation) can also aid in determining if a subject is likely to have cancer or in making a diagnosis of cancer.

The compositions and methods described herein can be administered to a subject having or diagnosed as having cancer. In some embodiments, the methods described herein comprise administering an effective amount of compositions described herein to a subject in order to alleviate a symptom of a cancer. As used herein, “alleviating a symptom of a cancer” is ameliorating any condition or symptom associated with the cancer. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of a compostion needed to alleviate at least one or more symptom of the disease or disorder, and relates to a sufficient amount of pharmacological composition to provide the desired effect. The term “therapeutically effective amount” therefore refers to an amount of a compostion that is sufficient to provide a particular anti-cancer effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of an active agent which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for SAKT signaling activity, among others. The dosage can be determined by a physician and adjusted, as necessary, to suit observed effects of the treatment.

In some embodiments, the technology described herein relates to a pharmaceutical composition as described herein, and optionally a pharmaceutically acceptable carrier. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, as described herein.

In some embodiments, the pharmaceutical composition as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of an agent as disclosed herein can also be incorporated into the parenteral dosage forms of the disclosure, including conventional and controlled-release parenteral dosage forms.

Pharmaceutical compositions can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos.: 3,845,770; 3,916,899; 3,536,809; 3,598,123; 4,008,719; 5674,533; 5,059,595; 5,591 ,767; 5,120,548; 5,073,543; 5,639,476; 5,354,556; 5,733,566; and 6,365,185 B1 ; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

The methods described herein can further comprise administering a second agent and/or treatment to the subject, e.g. as part of a combinatorial therapy. Non-limiting examples of a second agent and/or treatment, when the goal is cancer treatment, can include radiation therapy, surgery, gemcitabine, cisplastin, paclitaxel, carboplatin, bortezomib, AMG479, vorinostat, rituximab, temozolomide, rapamycin, ABT-737, PI-103; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKO polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE.RTM. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb.RTM.); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above. In addition, the methods of treatment can further include the use of radiation or radiation therapy. Further, the methods of treatment can further include the use of surgical treatments.

In certain embodiments, an effective dose of a composition as described herein can be administered to a patient once. In certain embodiments, an effective dose of a composition can be administered to a patient repeatedly. For systemic administration, subjects can be administered a therapeutic amount of a composition, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, or 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the composition. The desired dose can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition can be administered over a period of time, such as over a 5 minute, 10 minute, 15 minute, 20 minute, or 25 minute period.

The dosage ranges for the administration of a composition according to the methods described herein depend upon, for example, the form of the active agent, its potency, and the extent to which symptoms, markers, or indicators of a condition described herein are desired to be reduced, for example the percentage reduction desired for, e.g. tumor growth or the extent to which, for example, wound healing is desired to be induced. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a composition in, e.g. the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. tumor growth, insulin resistance, etc. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. tumor size and/or rate of growth). It is well within the ability of one skilled in the art to monitor efficacy of administration and/or treatment by measuring any one of such parameters, or any combination of parameters. Efficacy can be assessed in animal models of a condition described herein, for example treatment of cancer, ischemic injury, or wound healing. When using an experimental animal model, efficacy of treatment is evidenced when a statistically significant change in a marker is observed.

In vitro and animal model assays are provided herein which allow the assessment of a given dose of a modulator SAKT activity and/or SAKT signaling activity. By way of non-limiting example, the effects of a dose of a modulator can be assessed by bone marrow transplantation (BMT) assays. A non-limiting example of a protocol for such an assay is as follows: BM cells from a donor (e.g. marked donor cells, such as BM cells from a C57BL/6 5-fluorouracil (5-FU)—injected donor) are transplanted into a murine recipient. The cells can be contacted with a modulator of SAKT activity and/or SAKT signaling activity prior to implantation in the recipient or the recipient can be administered the modulator during or after transplantation. Peripheral blood analyses can be used to monitor hematopoietic reconstitution of donor cells in recipient mice, e.g., as compared to control donor cells. Hematopoietic reconstitution can be measured, e.g. 20 weeks after transplantation.

The efficacy of a given dosage combination can also be assessed in an animal model, e.g. a murine model of cancer.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of, e.g. cancer. A subject can be male or female.

A subject can be one who has been previously diagnosed with or identified as suffering from or having a condition in need of treatment (e.g. cancer) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene (e.g. mRNA), and polypeptides obtained by translation of mRNA transcribed from a gene.

The term “agent” refers generally to any entity which is normally not present or not present at the levels being administered to a cell. An agent can be selected from a group comprising: polynucleotides; polypeptides; small molecules; antibodies; or functional fragments thereof. As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight more than about 50, but less than about 5000 Daltons (5 kD). Preferably the small molecule has a molecular weight of less than 3 kD, still more preferably less than 2 kD, and most preferably less than 1 kD. In some cases it is preferred that a small molecule have a molecular mass equal to or less than 700 Daltons.

Inhibitors of the expression of a given gene, e.g. SAKT, can be an inhibitory nucleic acid. In some embodiments, the inhibitory nucleic acid is an inhibitory RNA (iRNA). Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part of a target mRNA transcript, e.g. of a SAKT mRNA transcript. The use of these iRNAs enables the targeted degradation of mRNA transcripts of SAKT, resulting in decreased expression and/or activity of SAKT. The following detailed description discloses how to make and use compositions containing iRNAs to inhibit the expression of SKAT, as well as compositions and methods for treating diseases and disorders caused by or modulated by the expression of SKAT, e.g. cancer.

In certain embodiments, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the mRNA level found in the cell without the presence of the iRNA.

In one aspect, an RNA interference agent includes a single stranded RNA that interacts with a target RNA sequence to direct the cleavage of the target RNA. Without wishing to be bound by theory, long double stranded RNA introduced into plants and invertebrate cells is broken down into siRNA by a Type III endonuclease known as Dicer (Sharp et al., Genes Dev. 2001, 15:485). Dicer, a ribonuclease-III-like enzyme, processes the dsRNA into 19-23 base pair short interfering RNAs with characteristic two base 3′ overhangs (Bernstein, et al., (2001) Nature 409:363). The siRNAs are then incorporated into an RNA-induced silencing complex (RISC) where one or more helicases unwind the siRNA duplex, enabling the complementary antisense strand to guide target recognition (Nykanen, et al., (2001) Cell 107:309). Upon binding to the appropriate target mRNA, one or more endonucleases within the RISC cleaves the target to induce silencing (Elbashir, et al., (2001) Genes Dev. 15:188). Thus, in one aspect the invention relates to a single stranded RNA that promotes the formation of a RISC complex to effect silencing of the target gene.

In some embodiments, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of SAKT. The other strand (the sense strand) includes a region that is complementary to the antisense strand, such that the two strands hybridize and form a duplex structure when combined under suitable conditions. Generally, the duplex structure is between 15 and 30 inclusive, more generally between 18 and 25 inclusive, yet more generally between 19 and 24 inclusive, and most generally between 19 and 21 base pairs in length, inclusive. Similarly, the region of complementarity to the target sequence is between 15 and 30 inclusive, more generally between 18 and 25 inclusive, yet more generally between 19 and 24 inclusive, and most generally between 19 and 21 nucleotides in length, inclusive. In some embodiments, the dsRNA is between 15 and 20 nucleotides in length, inclusive, and in other embodiments, the dsRNA is between 25 and 30 nucleotides in length, inclusive. As the ordinarily skilled person will recognize, the region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

One of skill in the art will also recognize that the duplex region is a primary functional portion of a dsRNA, e.g., a duplex region of 9 to 36, e.g., 15-30 base pairs. Thus, in one embodiment, to the extent that it becomes processed to a functional duplex of e.g., 15-30 base pairs that targets a desired RNA for cleavage, an RNA molecule or complex of RNA molecules having a duplex region greater than 30 base pairs is a dsRNA. Thus, an ordinarily skilled artisan will recognize that in one embodiment, then, an miRNA is a dsRNA. In another embodiment, a dsRNA is not a naturally occurring miRNA. In another embodiment, an iRNA agent useful to target SAKT expression is not generated in the target cell by cleavage of a larger dsRNA.

While a target sequence is generally 15-30 nucleotides in length, there is wide variation in the suitability of particular sequences in this range for directing cleavage of any given target RNA. Various software packages and the guidelines set out herein provide guidance for the identification of optimal target sequences for any given gene target, but an empirical approach can also be taken in which a “window” or “mask” of a given size (as a non-limiting example, 21 nucleotides) is literally or figuratively (including, e.g., in silico) placed on the target RNA sequence to identify sequences in the size range that may serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with an iRNA agent, mediate the best inhibition of target gene expression.

A dsRNA as described herein can further include one or more single-stranded nucleotide overhangs. The dsRNA can be synthesized by standard methods known in the art as further discussed below, e.g., by use of an automated DNA synthesizer, such as are commercially available from, for example, Biosearch, Applied Biosystems, Inc. In one embodiment, the antisense strand of a dsRNA has a 1-10 nucleotide overhang at the 3′ end and/or the 5′ end. In one embodiment, the sense strand of a dsRNA has a 1-10 nucleotide overhang at the 3′ end and/or the 5′ end. In one embodiment, at least one end of a dsRNA has a single-stranded nucleotide overhang of 1 to 4, generally 1 or 2 nucleotides. dsRNAs having at least one nucleotide overhang have unexpectedly superior inhibitory properties relative to their blunt-ended counterparts.

In another embodiment, one or more of the nucleotides in the overhang is replaced with a nucleoside thiophosphate. As used herein, the term “nucleotide overhang” refers to at least one unpaired nucleotide that protrudes from the duplex structure of an iRNA, e.g., a dsRNA. For example, when a 3′-end of one strand of a dsRNA extends beyond the 5′-end of the other strand, or vice versa, there is a nucleotide overhang. A dsRNA can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog, including a deoxynucleotide/nucleoside. The overhang(s) may be on the sense strand, the antisense strand or any combination thereof. Furthermore, the nucleotide(s) of an overhang can be present on the 5′ end, 3′ end or both ends of either an antisense or sense strand of a dsRNA.

The terms “blunt” or “blunt ended” as used herein in reference to a dsRNA mean that there are no unpaired nucleotides or nucleotide analogs at a given terminal end of a dsRNA, i.e., no nucleotide overhang. One or both ends of a dsRNA can be blunt. Where both ends of a dsRNA are blunt, the dsRNA is said to be blunt ended. To be clear, a “blunt ended” dsRNA is a dsRNA that is blunt at both ends, i.e., no nucleotide overhang at either end of the molecule. Most often such a molecule will be double-stranded over its entire length.

In specific embodiments, the iRNA comprises a single strand comprising a sequence, e.g. a 21-mer, comprised by the sequence of SEQ ID NO: 2. In some embodiments, the iRNA comprises a sequence, e.g. a 21-mer, comprised by the sequence of SEQ ID NO: 2.

In some embodiments, the one strand of the iRNA comprises and/or consists of a sequence comprised by the sequence of SEQ ID NO: 2 and the second strand comprises and/or consists of a nucleic acid sequence complementary to the first strand, e.g. at least the portion of the first strand. In this aspect, one of the two sequences is complementary to the other of the two sequences, with one of the sequences being substantially complementary to a sequence of a SAKT mRNA. As such, in this aspect, a dsRNA will include two oligonucleotides, where one oligonucleotide is described as the sense strand and the second oligonucleotide is described as the corresponding antisense strand of the sense strand. As described elsewhere herein and as known in the art, the complementary sequences of a dsRNA can also be contained as self-complementary regions of a single nucleic acid molecule, as opposed to being on separate oligonucleotides.

The skilled person is well aware that dsRNAs having a duplex structure of between 20 and 23, but specifically 21, base pairs have been hailed as particularly effective in inducing RNA interference (Elbashir et al., EMBO 2001, 20:6877-6888). However, others have found that shorter or longer RNA duplex structures can be effective as well. In the embodiments described above, dsRNAs described herein can include at least one strand of a length of minimally 21 nt. It can be reasonably expected that shorter duplexes minus only a few nucleotides on one or both ends may be similarly effective as compared to the dsRNAs described above. Hence, dsRNAs having a partial sequence of at least 15, 16, 17, 18, 19, 20, or more contiguous nucleotides, and differing in their ability to inhibit the expression of SAKT by not more than 5, 10, 15, 20, 25, or 30% inhibition from a dsRNA comprising a full 21-mer sequence, are contemplated according to the invention.

Further, it is contemplated that for any iRNA sequence, further optimization could be achieved by systematically either adding or removing nucleotides to generate longer or shorter sequences and testing those and sequences generated by walking a window of the longer or shorter size up or down the target RNA from that point. Again, coupling this approach to generating new candidate targets with testing for effectiveness of iRNAs based on those target sequences in an inhibition assay as known in the art or as described herein can lead to further improvements in the efficiency of inhibition. Further still, such optimized sequences can be adjusted by, e.g., the introduction of modified nucleotides as described herein or as known in the art, addition or changes in overhang, or other modifications as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes, etc.) as an expression inhibitor.

An iRNA as described herein can contain one or more mismatches to the target sequence. In one embodiment, an iRNA as described herein contains no more than 3 mismatches. If the antisense strand of the iRNA contains mismatches to a target sequence, it is preferable that the area of mismatch not be located in the center of the region of complementarity. If the antisense strand of the iRNA contains mismatches to the target sequence, it is preferable that the mismatch be restricted to be within the last 5 nucleotides from either the 5′ or 3′ end of the region of complementarity. For example, for a 23 nucleotide iRNA agent RNA strand which is complementary to a region of, e.g. SAKT, the RNA strand generally does not contain any mismatch within the central 13 nucleotides. The methods described herein or methods known in the art can be used to determine whether an iRNA containing a mismatch to a target sequence is effective in inhibiting the expression of, e.g. SAKT. Consideration of the efficacy of iRNAs with mismatches in inhibiting expression of the target gene is important, especially if the particular region of complementarity in the target gene is known to have polymorphic sequence variation within the population.

In yet another embodiment, the RNA of an iRNA, e.g., a dsRNA, is chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6, 239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. No. RE39464, each of which is herein incorporated by reference

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other RNA mimetics suitable or contemplated for use in iRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found, for example, in Nielsen et al., Science, 1991, 254, 1497-1500.

Some embodiments featured in the invention include RNAs with phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂— [wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs can also contain one or more substituted sugar moieties. The iRNAs, e.g., dsRNAs, featured herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Exemplary suitable modifications include O[(CH₂)ₙO]ₘCH₃, O(CH₂).ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e 2′-O—CH₂—O—CH₂—N(CH₂)₂, also described in examples herein below.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference.

An iRNA can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference, and U.S. Pat. No. 5,750,692, also herein incorporated by reference.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193).

Representative U.S. Patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845, each of which is herein incorporated by reference in its entirety.

Another modification of the RNA of an iRNA featured in the invention involves chemically linking to the RNA one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

In one embodiment, a ligand alters the distribution, targeting or lifetime of an iRNA agent into which it is incorporated. In preferred embodiments a ligand provides an enhanced affinity for a selected target, e.g, molecule, cell or cell type, compartment, e.g., a cellular or organ compartment, tissue, organ or region of the body, as, e.g., compared to a species absent such a ligand. Preferred ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a liver or tumor cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

Other examples of ligands include dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine)and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a cancer cell, hematopoietic cell, or epithelial cell. For example, the targeting ligand may specifically or non-specifically bind with a molecule on the surface of a target cell. The targeting moiety can be a molecule with a specific affinity for a target cell. Targeting moieties can include antibodies directed against a protein found on the surface of a target cell, or the ligand or a receptor-binding portion of a ligand for a molecule found on the surface of a target cell. For example, the targeting moiety can recognize a cancer-specific antigen (e.g ., CA15-3, CA19-9, CEA, or HER2/neu), thus delivering the iRNA to a cancer cell. Cancer-specific antigens and other means of targeting drugs specifically to cancer cells are known in the art. See, e.g. Wang et al. Ann Rev of Med 2012 63:185-198; Hutchinson Nature Rev Can 2005 5:759; Oh and Park Ad Drug Deliv Rev 2009 61:850-862; and Firer and Gellerman J Hemtol Oncol 2012 5:70; each of which is incorporated by reference herein in its entirety.

Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose. The ligand can be a substance, e.g, a drug, which can increase the uptake of the iRNA agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments, a ligand attached to an iRNA as described herein acts as a pharmacokinetic (PK) modulator. As used herein, a “PK modulator” refers to a pharmacokinetic modulator. PK modulators include lipophiles, bile acids, steroids, phospholipid analogues, peptides, protein binding agents, PEG, vitamins etc., that when linked to a composition, modify the body's metabolism of the composition, e.g. prolonging circulating half-life or otherwise stabilizing the composition, or, for example, resulting in accumulation of the composition in a locale different than that to which the composition locates in the absence of the PK modulator. Examplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that comprise a number of phosphorothioate linkages are also known to bind to serum protein, thus short oligonucleotides, e.g., oligonucleotides of about 5 bases, 10 bases, 15 bases or 20 bases, comprising multiple of phosphorothioate linkages in the backbaone are also amenable to the present invention as ligands (e.g. as PK modulating ligands). In addition, aptamers that bind serum components (e.g. serum proteins) are also suitable for use as PK modulating ligands in the embodiments described herein.

As used herein, the term “ischemic injury” refers to conditions directly associated with reduced blood flow to tissue, for example due to a clot or obstruction of blood vessels which supply blood to the subject tissue and which result, inter alia, in lowered oxygen transport to such tissue, impaired tissue performance, tissue dysfunction and/or necrosis and can contribute to the pathogenesis of heart failure. Alternatively, where blood flow or organ perfusion may be quantitatively adequate, the oxygen carrying capacity of the blood or organ perfusion medium may be reduced, e.g., in hypoxic environment, such that oxygen supply to the tissue is lowered, and impaired tissue performance, tissue dysfunction, and/or tissue necrosis ensues. “Ischemia/reperfusion injury” refers to a subset of ischemic injury in which injury involves a period of reduced blood flow, followed by at least partial restoration of the blood flow. Ischemia/reperfusion injury involves an inflammatory response and oxidative damage accompanied by apoptosis that occur when blood flow has been restored to a tissue subjected to an interruption in blood flow. As used herein, the term “ischemic limb disease” refers to any disease resulting from lack of blood flow to a superficial limb or extremity (e.g., an arm, leg, hand, foot, toe, finger etc.). Ischemic limb disease results from complications due to diabetes or atherosclerosis, among others.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. cancer. The term “treating” includes reducing or alleviating at least one adverse effect or symptom of a condition, disease or disorder associated with a condition. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

A “cancer” or “tumor” as used herein refers to an uncontrolled growth of cells which interferes with the normal functioning of the bodily organs and systems. A subject that has a cancer or a tumor is a subject having objectively measurable cancer cells present in the subject's body. Included in this definition are benign and malignant cancers, as well as dormant tumors or micrometastatses. Cancers which migrate from their original location and seed vital organs can eventually lead to the death of the subject through the functional deterioration of the affected organs.

A “cancer cell ” refers to a cancerous, pre-cancerous or transformed cell, either in vivo, ex vivo, and in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage dependence, proliferation, malignancy, contact inhibition and density limitation of growth, growth factor or serum dependence, tumor specific markers levels, invasiveness, tumor growth or suppression in suitable animal hosts such as nude mice, and the like, in vitro, in vivo, and ex vivo (see Example VII) (see also Freshney, Culture of Animal Cells: A Manual of Basic Technique (3rd ed. 1994)).

As used herein, the terms “chemotherapy” or “chemotherapeutic agent” refer to any chemical agent with therapeutic usefulness in the treatment of diseases characterized by abnormal cell growth. Such diseases include tumors, neoplasms and cancer as well as diseases characterized by hyperplastic growth. Chemotherapeutic agents as used herein encompass both chemical and biological agents. These agents function to inhibit a cellular activity upon which the cancer cell depends for continued survival. Categories of chemotherapeutic agents include alkylating/alkaloid agents, antimetabolites, hormones or hormone analogs, and miscellaneous antineoplastic drugs. Most, if not all, of these agents are directly toxic to cancer cells and do not require immune stimulation. In one embodiment, a chemotherapeutic agent is an agent of use in treating neoplasms, e.g. such as solid tumors. In one embodiment, a chemotherapeutic agent is a radioactive molecule (e.g. At²¹¹, I¹³¹, I¹²⁵, Y⁹⁰, Re¹⁸⁶, Re¹⁸⁸, Sm¹⁵³, Bi²¹², P³² and radioactive isotopes of Lu). One of skill in the art can readily identify a chemotherapeutic agent of use (e.g. see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993).

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); or Methods in Enzymology: Guide to Molecular Cloning Techniques Vol.152, S. L. Berger and A. R. Kimmel Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of treating a subject having cancer, the method comprising:
   determining, in a cancer cell sample obtained from the subject, the level of SAKT signaling activity; and
   administering a treatment to the subject;
   wherein a subject with a decreased level of SAKT signaling activity is administered a treatment comprising a therapeutically effective amount of an agonist of SAKT signaling; and
   wherein a subject with an increased level of SAKT signaling activity, as compared to a reference level, is administered a treatment comprising a therapeutically effective amount of an inhibitor of SAKT signaling.
2. A method of determining whether a cancer patient would benefit from treatment with an agonist of SAKT signaling, the method comprising:
   determining the level of SAKT signaling activity in a cancer cell sample obtained from the subject;
   wherein an agonist of SAKT signaling is indicated as an appropriate treatment if the level of SAKT signaling activity is decreased relative to a reference; and
   wherein an agonist of SAKT signaling is not indicated as an appropriate treatment if the level of SAKT signaling activity is not decreased relative to a reference.
3. A method of determining whether a cancer patient would benefit from treatment with an inhibitor of SAKT signaling, the method comprising:
   determining the level of SAKT signaling activity in a cancer cell sample obtained from the subject;
   wherein an inhibitor of SAKT signaling is indicated as an appropriate treatment if the level of SAKT signaling is increased relative to a reference; and
   wherein an inhibitor of SAKT signaling is not indicated as an appropriate treatment if the level of SAKT signaling activity is not increased relative to a reference.
4. The method of any of paragraphs 1-3, wherein increased SAKT signaling activity is determined by detecting a decreased level of AKT expression products and, optionally, an increased level of FOXO expression product.
5. The method of any of paragraphs 1-3, wherein decreased SAKT signaling activity is determined by detecting an increased level of AKT expression products.
6. The method of any of paragraphs 4-5, wherein the AKT expression products comprise phosphorylated AKT expression products.
7. The method of any of paragraphs 4-6, wherein the AKT expression products consist of phosphorylated AKT expression products.
8. The method of any of paragraphs 1-7, wherein increased SAKT signaling activity is determined by detecting a marker selected from the group consisting of:
   decreased levels of RICTOR; decreased levels of FOXO phosphorylation; decreased levels of FOXO3 phosphorylated at S253 and/or T32; decreased levels of mTOR phosphorylated at S2448; decreased levels of S6 phosphorylated at S235 and/or S236, decreased levels of 4EBP1 phosphorylated at T37 and/or T46; decreased levels of PRAS40 phosphorylated at T246; decreased levels of phosphorylated STAT3; decreased levels of phosphorylated SGK; decreased levels of SGK phosphorylated at S422; decreased levels of phosphorylated PKCα; decreased levels of PKCα phosphorylated at S638; and decreased levels of AKT phosphorylated at S473.
9. The method of any of paragraphs 1-7, wherein decreased SAKT signaling activity is determined by detecting a marker selected from the group consisting of:
   increased levels of RICTOR; increased levels of FOXO phosphorylation; increased levels of FOXO3 phosphorylated at S253 and/or T32; increased levels of mTOR phosphorylated at S2448; increased levels of S6 phosphorylated at S235 and/or S236; increased levels of 4EBP1 phosphorylated at T37 and/or T46; increased levels of PRAS40 phosphorylated at T246; increased levels of phosphorylated STAT3; increased levels of phosphorylated SGK; increased level of SGK phosphorylated at S422; increased levels of phosphorylated PKCα; increased levels of PKCα phosphorylated at S638; and increased levels of AKT phosphorylated at S473.
10. The method of any of paragraphs 1-9, wherein the agonist of SAKT signaling is selected from the group consisting of:
   an agonist of SAKT; an inhibitor of RICTOR; an inhibitor of mTORC2; and an inhibitor of RICTOR-mTORC2 interaction.
11. The method of paragraph 10, wherein the agonist of SAKT is selected from the group consisting of:
   an antibody reagent agonist; and a nucleic acid encoding SAKT.
12. The method of any of paragraphs 1-9, wherein the inhibitor of SAKT signaling is selected from the group consisting of:
   an inhibitor of SAKT; an agonist of RICTOR; an agonist of mTORC2; and an agonist of RICTOR-mTORC2 interaction.
13. The method of paragraph 12, wherein the inhibitor of SAKT is selected from the group consisting of:
   an inhibitory nucleic acid molecule; and an antibody reagent.
14. The method of any of paragraphs 1-13, wherein the cell is selected from the group consisting of:
   a hematopoietic cancer cell and an epithelial cancer cell.
15. The method of paragraph 14, wherein the epithelial cancer cell is selected from the group consisting of:
   carcinoma; adenocarcinoma; basal cell carcinoma; squamous cell carcinoma; large cell carcinoma; small cell carcinoma; colorectal adenocarcinoma; lung cancer; breast cancer; prostate cancer; colon cancer; rectal cancer; pancreatic cancer; kidney cancer; ovarian cancer; stomach cancer; intestinal cancer; oral cancer; esophageal cancer; lip cancer; bladder cancer; cervical cancer; skin cancer; hepatocellular carcinoma; and renal cell carcinoma.
16. The method of any of paragraphs 1-15, wherein the level of AKT or Foxo expression products is determined by measuring the level of RNA transcripts.
17. The method of paragraph 16, wherein the RNA transcript level is measured using reverse transcription polymerase chain reaction (RT-PCR).
18. The method of any of paragraphs 1-15, wherein the level of AKT and Foxo expression products is determined by measuring the level of polypeptides.
19. The method of paragraph 18, wherein the polypeptide level is measured using immunochemistry.
20. The method of paragraph 19, wherein the immunochemical method comprises:
   (a) contacting a biofluid test sample obtained from a subject with a detectable anti-AKT antibody reagent and optionally, an anti-Foxo antibody reagent; and
   (b) detecting the presence or intensity of a detectable signal;
   wherein the expression level of AKT polypeptide, and optionally, Foxo polypeptide, is indicated by the level of the detectable signal.
21. The method of any of paragraphs 20, wherein the antibody reagent is detectably labeled or capable of generating a detectable signal.
22. The method of any of paragraphs 1-21, wherein the sample comprises a material selected from the group consisting of:
   blood or a product thereof; serum; plasma; and a tumor biopsy.
23. The method of any of paragraphs 1-22, wherein the level of a marker of SAKT signaling activity, is normalized relative to the expression level of one or more reference genes or reference proteins.
24. The method of any of paragraphs 1-23, wherein the reference expression level is the expression level in a sample obtained from a subject not having cancer.
25. The method of any of paragraphs 1-23, wherein the reference expression level is the expression level in a prior sample obtained from the subject.
26. The method of any of paragraphs 1-25, wherein an increased level is a level at least 25% greater than a reference level.
27. The method of any of paragraphs 1-25, wherein a decreased level is a level at least 25% less than a reference level.
28. The method of any of paragraphs 1-27, wherein the expression level of no more than 20 other genes is determined.
29. The method of any of paragraphs 1-28, wherein the expression level of no more than 10 other genes is determined.
30. The method of any of paragraphs 1-29, wherein the subject is a human.
31. An assay comprising:
   (a) contacting a cancer cell sample obtained from a subject with a detectable anti-AKT antibody reagent; and
   (b) detecting the presence or intensity of a detectable signal;
   wherein an increase in the level of AKT polypeptide, indicated by the level of the detectable signal, relative to a reference level indicates the subject is in need of treatment with an agonist of SAKT signaling activity; and
   wherein a decrease in the level of AKT polypeptide, indicated by the level of the detectable signal, relative to a reference level indicates the subject is in need of treatment with an inhibitor of SAKT signaling activity.
32. The assay of paragraph 31, further comprising contacting the cancer cell sample with a detectable anti-Foxo antibody reagent
33. The assay of any of paragraphs 31-32, wherein the anti-AKT antibody reagent is specific for AKT polyeptide phosphorylated at S473.
34. The assay of any of paragraphs 31-33, wherein the agonist of SAKT signaling is selected from the group consisting of:
   an agonist of SAKT; an inhibitor of RICTOR; an inhibitor of mTORC2; and an inhibitor of RICTOR-mTORC2 interaction.
35. The assay of paragraph 34, wherein the agonist of SAKT is selected from the group consisting of:
   an antibody reagent agonist; and a nucleic acid encoding SAKT.
36. The assay of any of paragraphs 31-33, wherein the inhibitor of SAKT signaling is selected from the group consisting of:
   an inhibitor of SAKT; an agonist of RICTOR; an agonist of mTORC2; and an agonist of RICTOR-mTORC2 interaction.
37. The method of paragraph 36, wherein the inhibitor of SAKT is selected from the group consisting of:
   an inhibitory nucleic acid molecule; and an antibody reagent.
38. The assay of any of paragraphs 31-37, wherein the cell is selected from the group consisting of:
   a hematopoietic cancer cell and an epithelial cancer cell.
39. The assay of paragraph 38, wherein the epithelial cancer cell is selected from the group consisting of:
   carcinoma; adenocarcinoma; basal cell carcinoma; squamous cell carcinoma; large cell carcinoma; small cell carcinoma; colorectal adenocarcinoma; lung cancer; breast cancer; prostate cancer; colon cancer; rectal cancer; pancreatic cancer; kidney cancer; ovarian cancer; stomach cancer; intestinal cancer; oral cancer; esophageal cancer; lip cancer; bladder cancer; cervical cancer; skin cancer; hepatocellular carcinoma; and renal cell carcinoma.
40. The assay of any of paragraphs 31-39, wherein the sample comprises a material selected from the group consisting of:
   blood or a product thereof; serum; plasma; and a tumor biopsy.
41. The assay of any of paragraphs 31-40, wherein the level AKT is normalized relative to the expression level of one or more reference genes or reference proteins.
42. The assay of any of paragraphs 31-41, wherein the reference expression level is the expression level in a sample obtained from a subject not having cancer.
43. The assay of any of paragraphs 31-42, wherein the reference expression level is the expression level in a prior sample obtained from the subject.
44. The assay of any of paragraphs 31-43, wherein an increased level is a level at least 25% greater than a reference level.
45. The assay of any of paragraphs 31-44, wherein a decreased level is a level at least 25% less than a reference level.
46. The assay of any of paragraphs 31-45, wherein the expression level of no more than 20 other genes is determined.
47. The assay of any of paragraphs 31-46, wherein the expression level of no more than 10 other genes is determined.
48. The assay of any of paragraphs 31-47, wherein the subject is a human.
49. A method of suppressing AKT activity, comprising administering an agonist of SAKT activity or expression.
50. A method of treating ischemic injury, the method comprising administering an agonist of SAKT signaling activity.
51. A method of altering the sensitivity of a cell to a growth factor, the method comprising;
   contacting the cell with an inhibitor of SAKT signaling activity to render the cell more sensitive to the growth factor; or
   contacting the cell with an agonist of SAKT signaling activity to render the cell less sensitive to the growth factor.
52. The method of paragraph 51, wherein the growth factor is selected from the group consisting of:
   insulin; epidermal growth factor (EGF); hematopoietic growth factor; vascular endothelial growth factor (VEGF); insulin-like growth factor-1 (IGF); platelet-derived growth factor (PDGF); granulocyte colony-stimulating factor (G-CSF); platelet activating factor; and macrophage stimulating factor.
53. The method of any of paragraphs 51-52, wherein the agonist or inhibitor of SAKT signaling activity is administered to a subject.
54. The method of paragraph 51-53, wherein the subject is in need of treatment for a condition selected from the group consisting of:
   insulin resistance; diabetes; cancer; proliferative diseases; and wound healing.
55. The method of any of paragraphs 48-54, wherein the agonist of SAKT signaling is selected from the group consisting of:
   an agonist of SAKT; an inhibitor of RICTOR; an inhibitor of mTORC2; and an inhibitor of RICTOR-mTORC2 interaction.
56. The method of paragraph 55, wherein the agonist of SAKT is selected from the group consisting of:
   an antibody reagent agonist; and a nucleic acid encoding SAKT.
57. The method of any of paragraphs 48-54, wherein the inhibitor of SAKT signaling is selected from the group consisting of:
   an inhibitor of SAKT; an agonist of RICTOR; an agonist of mTORC2; and an agonist of RICTOR-mTORC2 interaction.
58. The method of paragraph 57, wherein the inhibitor of SAKT is selected from the group consisting of:
   an inhibitory nucleic acid molecule; and an antibody reagent.
59. A computer system for determining the appropriate treatment for a subject having cancer, the system comprising:
   a measuring module configured to measure the level of SAKT signaling activity, in a test sample obtained from a subject;
   a storage module configured to store output data from the determination module;
   a comparison module adapted to compare the data stored on the storage module with a reference level, and to provide a retrieved content, and
   a display module for displaying whether the sample comprises a level of SAKT signaling activity which is significantly increased or decreased relative to the reference level and/or displaying the relative SAKT signaling activity.
60. The system of paragraph 59, wherein the measuring module measures the intensity of a detectable signal from an assay indicating the level of a polypeptide marker of SAKT signaling activity in the test sample.
61. The system of paragraph 60, wherein the assay is an immunoassay.
62. The system of any of paragraphs 59-61, wherein if the computing module determines that the level of SAKT signaling activity in the test sample obtained from a subject is less by a statistically significant amount than the reference level, the display module displays a signal indicating that the expression levels in the sample obtained from a subject are less than those of the reference level;
63. The system of any of paragraphs 59-62, wherein if the computing module determines that the level of SAKT signaling activity in the test sample obtained from a subject is less by a statistically significant amount than the reference level, the display module displays a signal indicating that the appropriate treatment for the subject is an agonist of SAKT signaling.
64. The system of any of paragraphs 59-63, wherein if the computing module determines that the level of SAKT signaling activity in the test sample obtained from a subject is greater by a statistically significant amount than the reference level, the display module displays a signal indicating that the expression levels in the sample obtained from a subject are greater than those of the reference level.
65. The system of any of paragraphs 59-64, wherein if the computing module determines that the level of SAKT signaling activity in the test sample obtained from a subject is greater by a statistically significant amount than the reference level, the display module displays a signal indicating that the appropriate treatment for the subject is an inhibitor of SAKT signaling.
66. The system of any of paragraphs 59-65, wherein the signal indicates the degree to which the level of SAKT signaling activity in the sample obtained from a subject varies from the reference level.
67. The system of any of paragraphs 59-66, wherein increased SAKT signaling activity is determined by detecting a decreased level of AKT expression products and, optionally, an increased level of FOXO expression product.
68. The system of any of paragraphs 59-67, wherein decreased SAKT signaling activity is determined by detecting an increased level of AKT expression products.
69. The system of any of paragraphs 59-68, wherein the AKT expression products comprise phosphorylated AKT expression products.
70. The system of any of paragraphs 59-69, wherein the AKT expression products consist of phosphorylated AKT expression products.
71. The system of any of paragraphs 59-70, wherein increased SAKT signaling activity is determined by detecting a marker selected from the group consisting of:
   decreased levels of RICTOR; decreased levels of FOXO phosphorylation; decreased levels of FOXO3 phosphorylated at S253 and/or T32; decreased levels of mTOR phosphorylated at S2448; decreased levels of S6 phosphorylated at S235 and/or S236, decreased levels of 4EBP1 phosphorylated at T37 and/or T46; decreased levels of PRAS40 phosphorylated at T246; decreased levels of phosphorylated STAT3; decreased levels of phosphorylated SGK; decreased levels of SGK phosphorylated at S422; decreased levels of phosphorylated PKCα; decreased levels of PKCα phosphorylated at S638; and decreased levels of AKT phosphorylated at S473.
72. The system of any of paragraphs 59-71, wherein decreased SAKT signaling activity is determined by detecting a marker selected from the group consisting of:
   increased levels of RICTOR; increased levels of FOXO phosphorylation; increased levels of FOXO3 phosphorylated at S253 and/or T32; increased levels of mTOR phosphorylated at S2448; increased levels of S6 phosphorylated at S235 and/or S236; increased levels of 4EBP1 phosphorylated at T37 and/or T46; increased levels of PRAS40 phosphorylated at T246; increased levels of phosphorylated STAT3; increased levels of phosphorylated SGK; increased level of SGK phosphorylated at S422; increased levels of phosphorylated PKCα; increased levels of PKCαphosphorylated at S638; and increased levels of AKT phosphorylated at S473.
73. The system of any of paragraphs 59-72, wherein the agonist of SAKT signaling is selected from the group consisting of:
   an agonist of SAKT; an inhibitor of RICTOR; an inhibitor of mTORC2; and an inhibitor of RICTOR-mTORC2 interaction.
74. The system of paragraph 73, wherein the agonist of SAKT is selected from the group consisting of:
   an antibody reagent agonist; and a nucleic acid encoding SAKT.
75. The system of any of paragraphs 59-74, wherein the inhibitor of SAKT signaling is selected from the group consisting of:
   an inhibitor of SAKT; an agonist of RICTOR; an agonist of mTORC2; and an agonist of RICTOR-mTORC2 interaction.
76. The system of paragraph 75, wherein the inhibitor of SAKT is selected from the group consisting of:
   an inhibitory nucleic acid molecule; and an antibody reagent.
77. The system of any of paragraphs 59-76, wherein the cell is selected from the group consisting of:
   a hematopoietic cancer cell and an epithelial cancer cell.
78. The system of paragraph 77, wherein the epithelial cancer cell is selected from the group consisting of:
   carcinoma; adenocarcinoma; basal cell carcinoma; squamous cell carcinoma; large cell carcinoma; small cell carcinoma; colorectal adenocarcinoma; lung cancer; breast cancer; prostate cancer; colon cancer; rectal cancer; pancreatic cancer; kidney cancer; ovarian cancer; stomach cancer; intestinal cancer; oral cancer; esophageal cancer; lip cancer; bladder cancer; cervical cancer; skin cancer; hepatocellular carcinoma; and renal cell carcinoma.
79. The system of any of paragraphs 59-78, wherein the level of AKT or Foxo expression products is determined by measuring the level of RNA transcripts.
80. The system of paragraph 79, wherein the RNA transcript level is measured using reverse transcription polymerase chain reaction (RT-PCR).
81. The system of paragraph 61, wherein the immunochemical method comprises:
   (a) contacting a biofluid test sample obtained from a subject with a detectable anti-AKT antibody reagent and optionally, an anti-Foxo antibody reagent; and
   (b) detecting the presence or intensity of a detectable signal;
   wherein the expression level of AKT polypeptide, and optionally, Foxo polypeptide, is indicated by the level of the detectable signal.
82. The system of any of paragraphs 60-61 and 81, wherein the antibody reagent is detectably labeled or capable of generating a detectable signal.
83. The system of any of paragraphs 59-82, wherein the sample comprises a material selected from the group consisting of:
   blood or a product thereof; serum; plasma; and a tumor biopsy.
84. The system of any of paragraphs 59-83, wherein the level of a marker of SAKT signaling activity, is normalized relative to the expression level of one or more reference genes or reference proteins.
85. The system of any of paragraphs 59-84, wherein the reference expression level is the expression level in a sample obtained from a subject not having cancer.
86. The system of any of paragraphs 59-85, wherein the reference expression level is the expression level in a prior sample obtained from the subject.
87. The system of any of paragraphs 59-86, wherein an increased level is a level at least 25% greater than a reference level.
88. The system of any of paragraphs 59-87, wherein a decreased level is a level at least 25% less than a reference level.
89. The system of any of paragraphs 59-88, wherein the expression level of no more than 20 other genes is determined.
90. The system of any of paragraphs 59-89, wherein the expression level of no more than 10 other genes is determined.

### EXAMPLES

### Example 1

### Novel Receptor Mediating Suppression of AKT Through Rictor/mTORC2

Central to cellular proliferative, survival and metabolic responses to cytokine signals is the serine/threonine kinase, AKT, whose activation is among the most common alterations in human cancer. Described herein is a novel cell membrane receptor that inhibits the activation of AKT; identified through the analysis of a mouse model of microenvironment-induced acute leukemia. This suppressor of AKT (SAKT) functions through the known AKT pathway component, mammalian target of rapamycin (mTOR) (1-4) that exists as two distinct multi-subunit protein complexes, mTORC1 and mTORC2 (5, 6). SAKT binds the mTORC2 subunit Rictor inhibiting mTORC2 activity. Modifying SAKT altered AKT activation in primary hematopoietic stem and progenitor cells and influenced their survival and differentiation in vitro and in vivo. These studies identify a previously unrecognized inhibitory component of the AKT-mTOR signaling network and uncover a means by which environmental cues may down-modulate the activity of this critical cell-regulatory pathway.

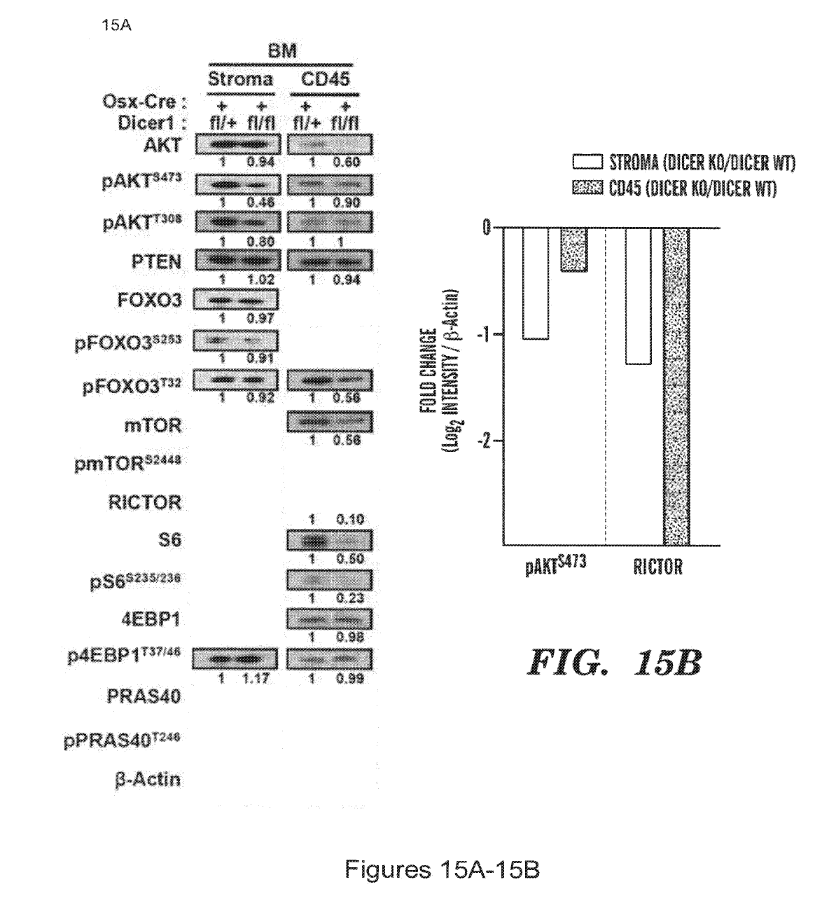

Genetic ablation of the small double-stranded RNA processing enzyme, Dicer, in ostrix-positive mesenchymal cells of the bone marrow (Osx-GFP-Cre⁺; Dicer^{fl/fl}) disrupts hematopoiesis and induces a myelodysplastic state (7). In a small number of animals, an acute myeloid leukemia (AML) emerged that involved complex, genetic lesions distinct from Dicer deletion in mesenchymal cells. Shared among two of these leukemias was an amplification of a region of mouse chromosome 14. Also noted in Osx-GFP-Cre⁺; Dicer^{fl/fl} mice were alterations in the AKT-mTOR signaling pathway previously implicated in AML (8). Specifically, AKT phosphorylation (pAKT^{Ser473} and pAKT^{Thr308}), S6 phosphorylation (pS6^{Ser235/236}) and mTOR phosphorylation (pmTor^{Ser2448}) were diminished upon Dicer deletion (FIGS. 15A-15B). Decreased activity of the AKT pathway is associated with differentiation arrest in ˜40% of human AML (8). To determine if the genotypic changes on chromosome 14 were related to altered AKT-mTOR signaling, the effect of the open reading frame C14ORF37 (NCBI gene ID 145407), contained on the chromosome 14 amplicon, on AKT-mTOR pathway constituents was examined.

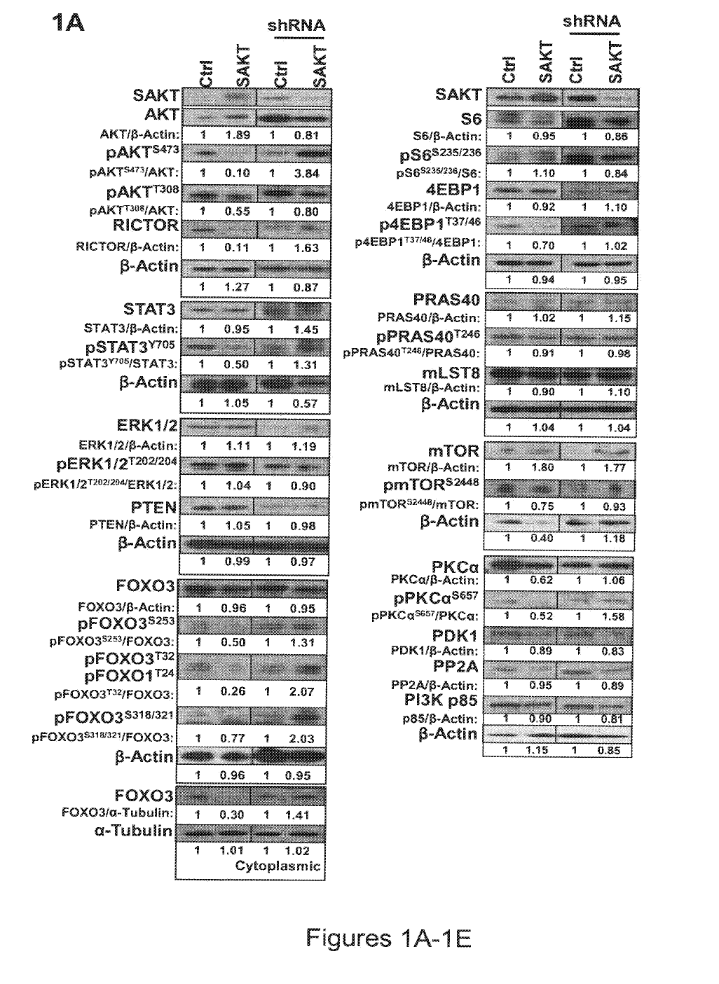

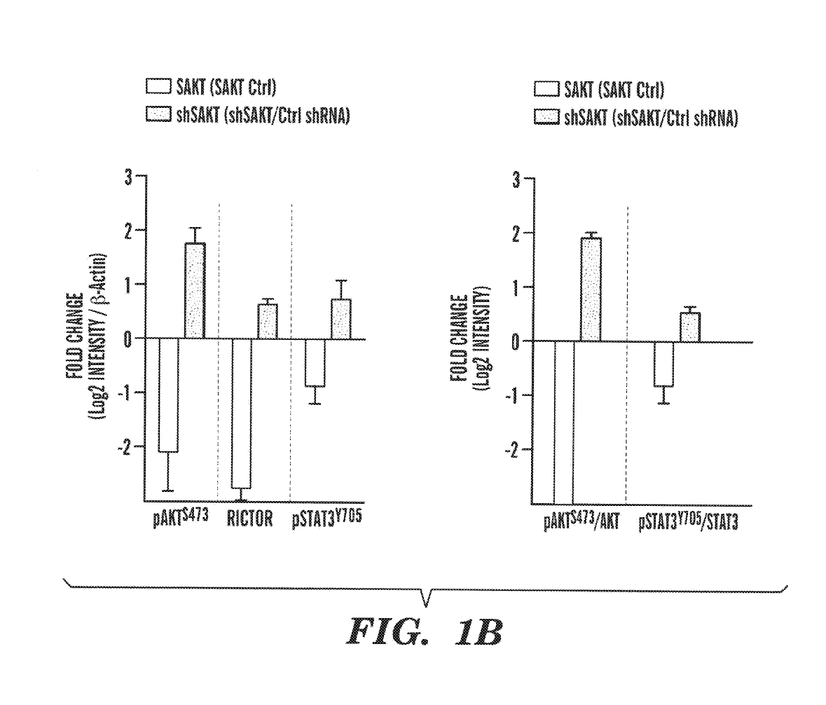

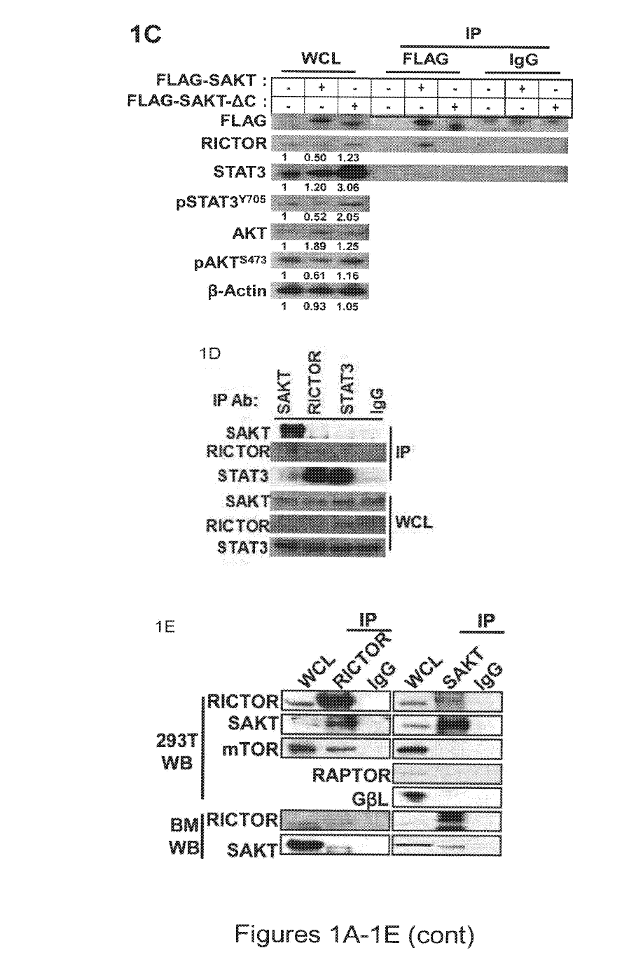

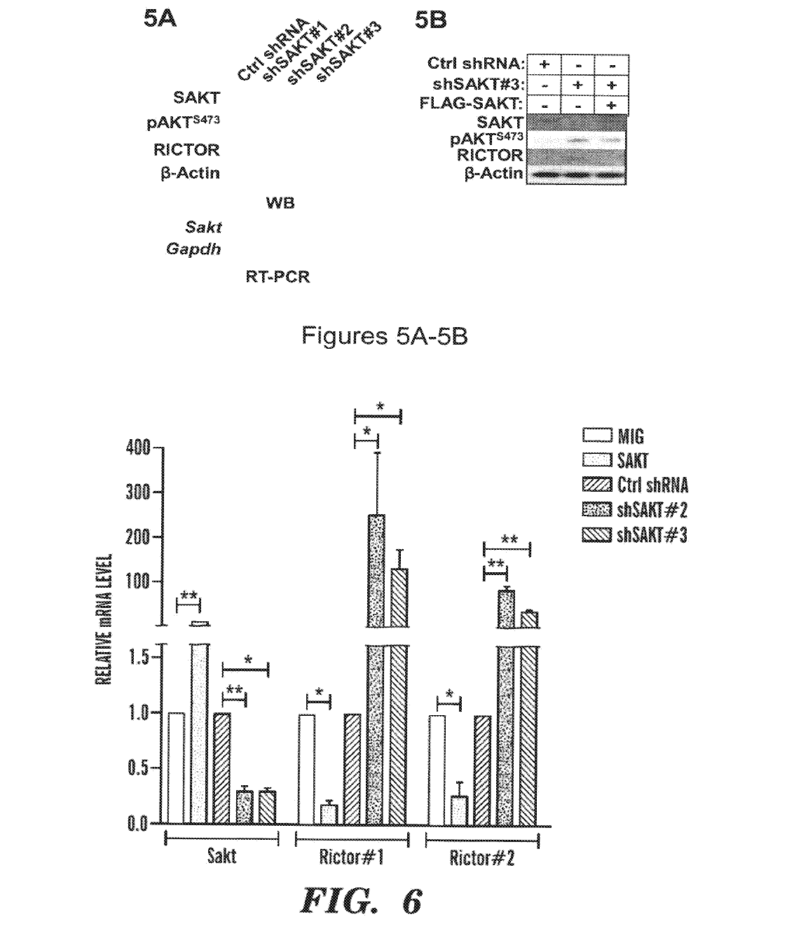

C14ORF37 encodes a putative type I transmembrane protein. Imaging of bone marrow (BM) cells stained with fluorescent-labeled C14ORF37 anti-sera confirmed that C14ORF37 is primarily localized to the cellular membrane on hematopoietic cells (data not shown). Enforced expression of C14ORF37 in murine BM cells resulted in decreased phosphorylation of AKT^{Ser473}, mTor^{Ser2448}, S6^{Ser235/236}, 4EBP1^{Thr37/46} and Pras40^{Thr246} as well as decreased Rictor levels (FIG. 1A, left panels). Conversely, shRNA-mediated inhibition of C14ORF37 expression increased Rictor and AKT^{Ser473}, mTor^{Ser2448}, S6^{Ser235/236}, 4EBP1^{Thr37/46} and Pras40^{Thr246} phosphorylation (FIG. 1A, right panels depicting results with one of three shRNAs targeting independent sites giving comparable results; data from other shRNAs shown in FIGS. 5A-5B). These data suggest a novel function of C14ORF37, namely modulating Rictor and the down-regulation of downstream mTORC2 components, including AKT. Therefore, C14ORF37 is designated herein as Suppressor of AKT (SAKT) in reference to its apparent impact on that important pathway.

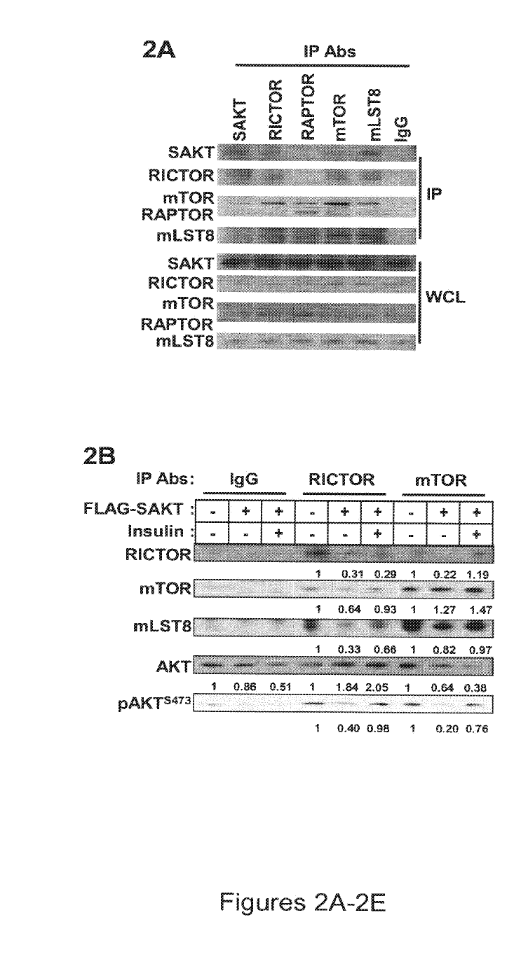

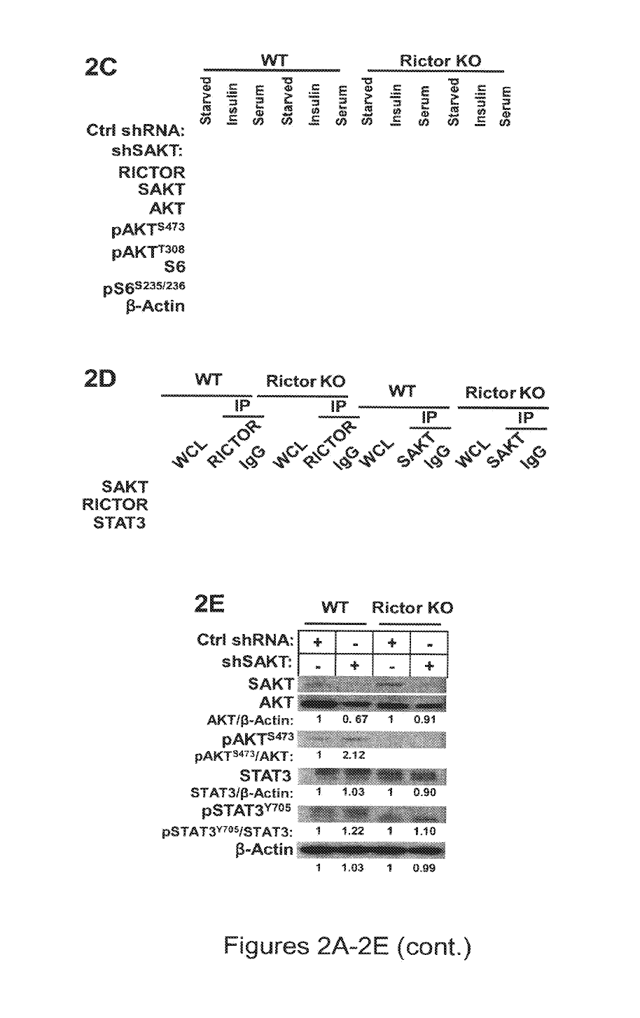

Strikingly, while altering SAKT expression had an impact on the phosphorylation of AKT^{Ser473}, AKT^{Thr308} phosphorylation remained largely unaffected (FIG. 1A). AKT^{Ser473} phosphorylation is catalyzed by the multi-subunit protein complex mTorc2. Therefore, whether the ability of SAKT to suppress AKT^{Ser473} phosphorylation is mTorc2-dependent was investigated. Consistent with previous studies, genetic ablation of the mTorc2 complex component Rictor abolished pAKT^{Ser473.} The ability of SAKT shRNA to modify AKT^{Ser473} was also blocked by the deletion of Rictor (FIG. 2C).

It was postulated that SAKT could regulate pAKT^{Ser473} phosphorylation through physical interaction with the mTorc2 complex. To address this, the cellular distribution of endogenous SAKT and Rictor was addressed by immunostaining. BM cells stained with SAKT and Rictor antibodies revealed that a substantial proportion of SAKT co-localized with Rictor (data not shown). Co-immunoprecipitation assays in both 293T and murine BM cells were performed and SAKT and Rictor co-precipitated from 293T or BM cell lysates incubated with either Rictor-specific or SAKT-specific antibodies, indicating that these two proteins interact. Of note, SAKT-specific anti-sera did not co-precipitate mTor, Raptor or GβL indicating that SAKT interacts with Rictor and not other components of the mTorc1 and mTorc2 complexes (FIG. 1E). Also, Rictor antibodies did not co-precipitate SAKT from cellular lysates where Rictor had been genetically ablated, arguing against non-specific binding of Rictor antibodies (FIG. 2C). To approximate the region of SAKT required for interaction with Rictor, full length or C-terminal deleted (aa 737-775) SAKT were separately expressed in 293T cells (FIG. 1C). Rictor interaction required the C-terminal portion of SAKT that encompass the cytoplasmic region. Taken together, these results indicate that SAKT modulates AKT activation through Rictor-dependent pathways.

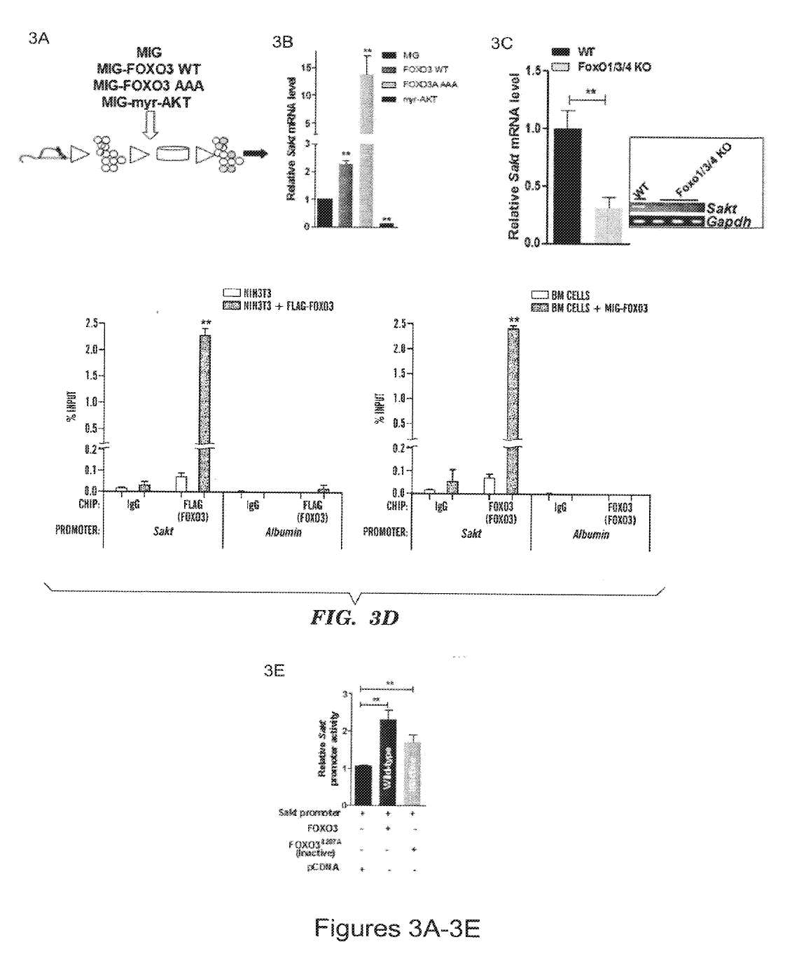

AKT is well-characterized as an oncogene that mediates cell survival, in part by directly phosphorylating and inhibiting the tumor suppresor FoxO3 (9, 10). The ability of AKT to regulate FoxO3 activity is highly dependent upon Rictor-catalyzed phosphorylation of AKT at serine 473 (11). Consistent with SAKT's ability to regulate Rictor expression, phosphorylation of FoxO3 at serine-253 (pFoxO3^{Ser253}) and threonine-32 (pFoxO3^{Thr32}) were modulated by SAKT expression (FIG. 1A). The AKT signaling pathway is regulated by a sophisticated network of regulatory feed-back and -forward loops. Therefore, whether FoxO3 could directly regulate SAKT expression as part of a feed-forward mechanism for dampening AKT signaling was examined. To address this, murine BM cells were transduced with recombinant retroviruses expressing FoxO3 WT, FoxO3 triple mutant (AAA, FoxO3 active form by AKT dephosphorylation) (9) or myristoylated AKT (myr-AKT, constitutive phosphorylation and activation form) (12, 13) (FIG. 3A). Enforced expression of either the wild-type or the AKT-resistant mutant version of FoxO3 activates SAKT expression (FIG. 3B). Additionally, constitutive activation of AKT drastically reduced SAKT expression (FIG. 3B). Confirming that members of the FoxO family regulate SAKT expression in vivo (FIG. 3C). BM cells derived from mice lacking functional purified FoxO1, FoxO3 and FoxO4 (hereafter FoxO1/3/4 KO) alleles displayed significantly lower levels of SAKT transcripts compared to wild type BM cells (FIG. 3C). Sequence analysis of the SAKT promoter showed that there is a putative FoxO3 binding site embedded approximately 7 kb upstream of the transcriptional start site. Furthermore, FoxO3 can activate luciferase expression driven by a minimal promoter containing the putative FoxO3 consensus sequence found in the SAKT promoter (FIG. 3E). Chromatin immunoprecipitation (ChIP) with FoxO3-specific antibodies in murine BM and fibroblast cells showed that FoxO3 binds to this conserved site within the SAKT promoter in vivo (FIG. 3D). Collectively, these data show that FoxO3 directly regulates the transcription of SAKT.

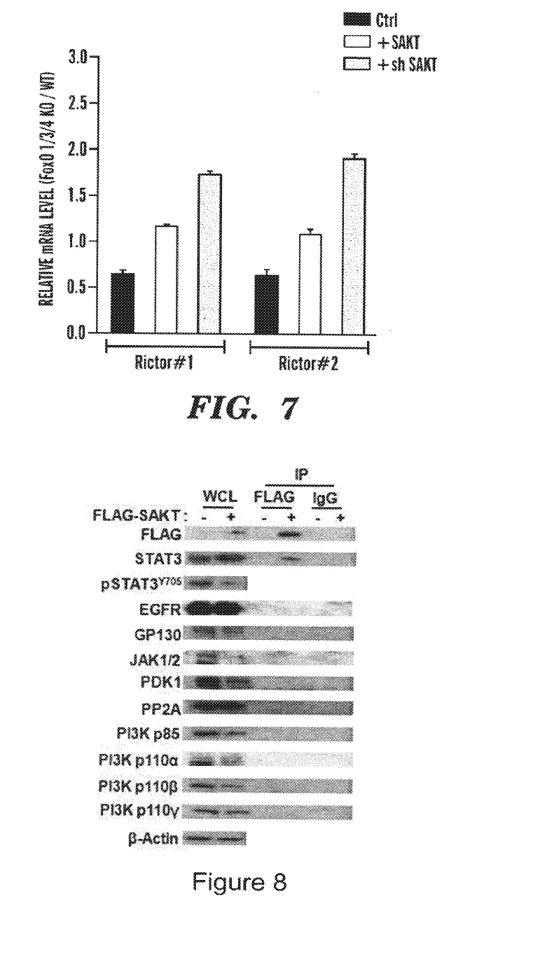

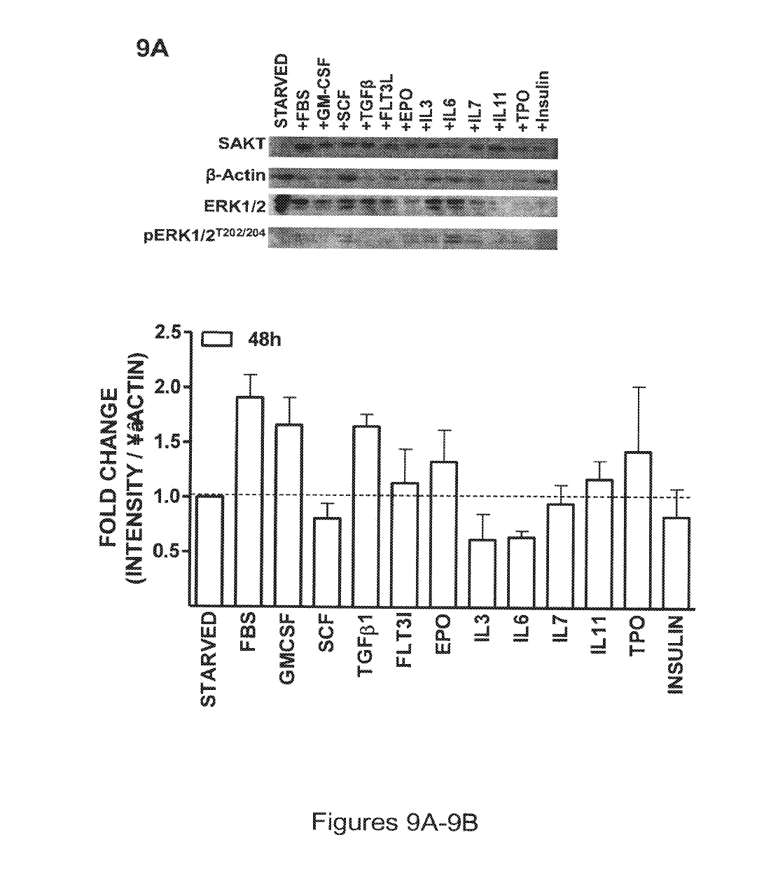

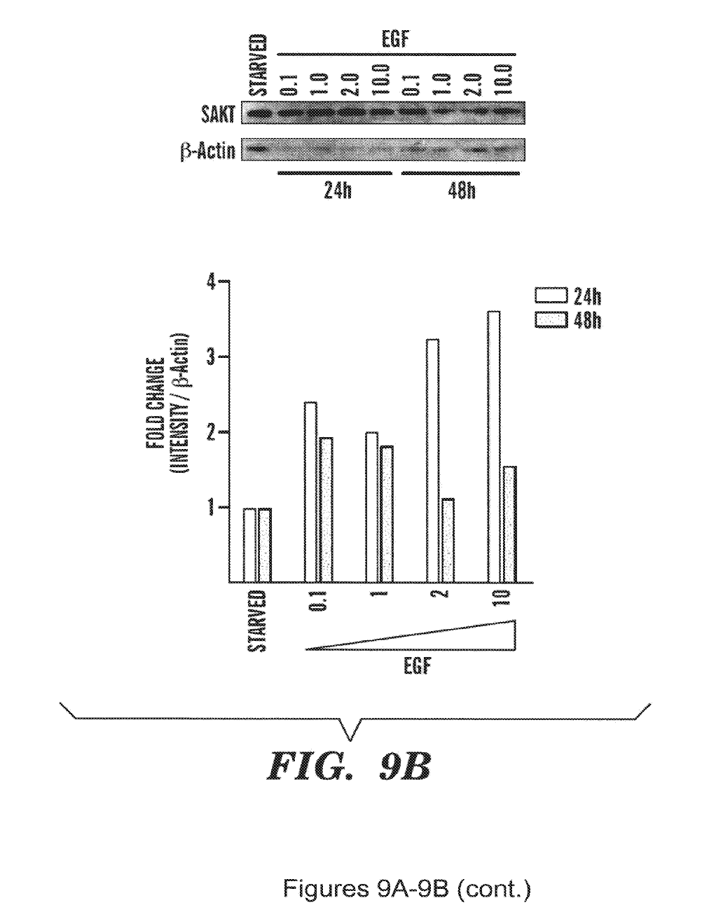

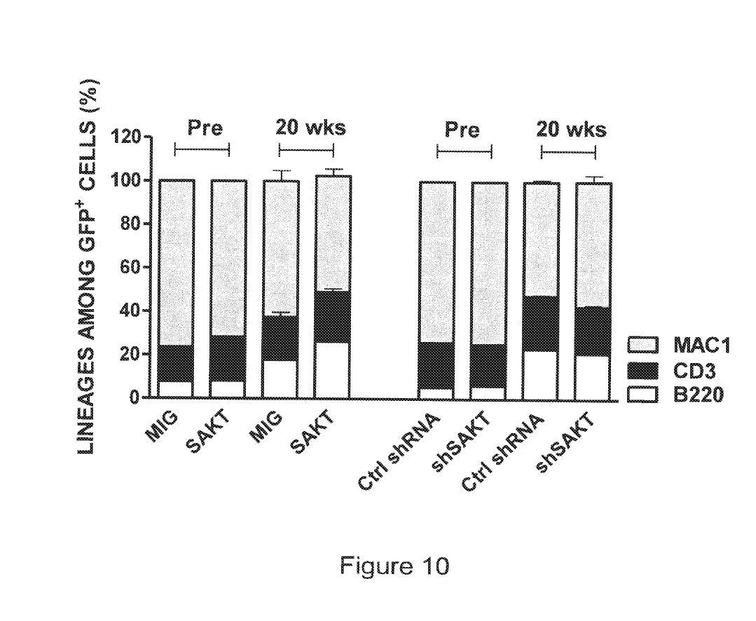

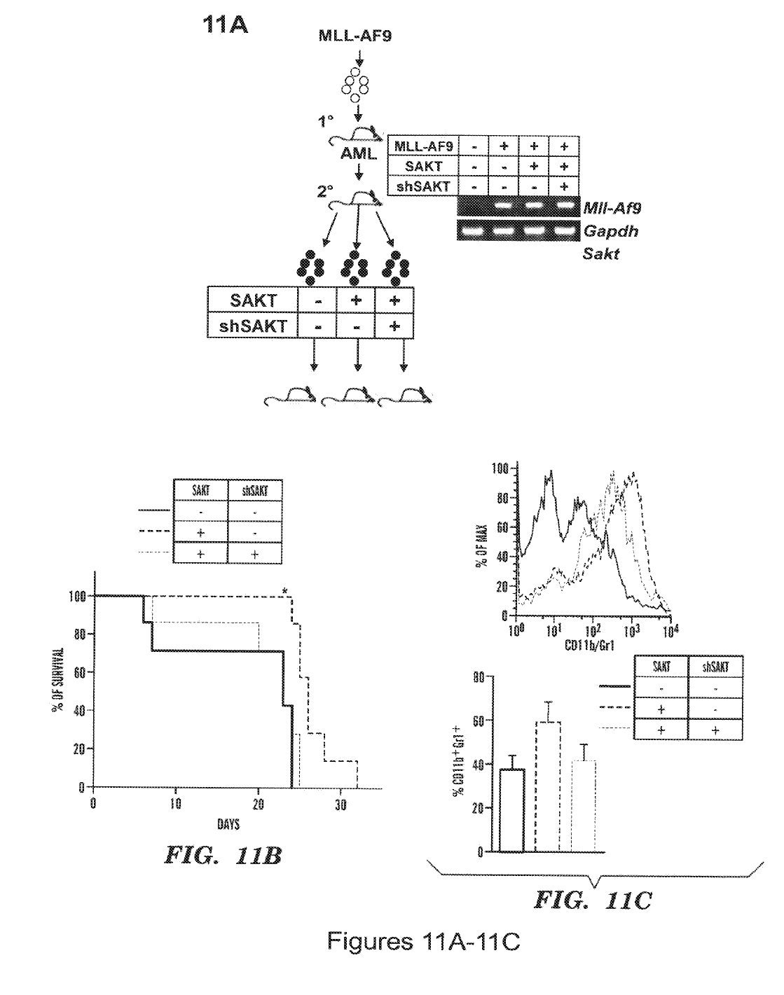

SAKT regulation of Rictor levels was evaluated and found to occur at the level of transcription (FIG. 6). It was therefore evaluated whether it too was mediated by FoxO3, but it was found that the downregulation of Rictor mRNA by SAKT was not affected by deletion of FoxO1/3/4 (FIG. 7).

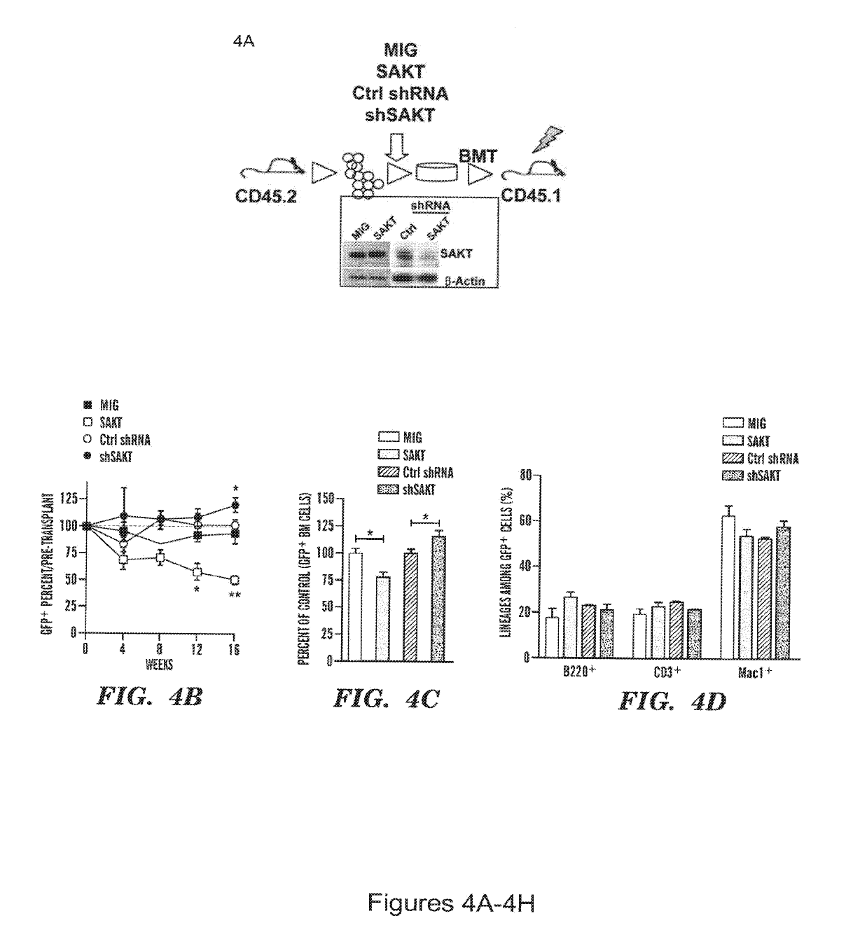

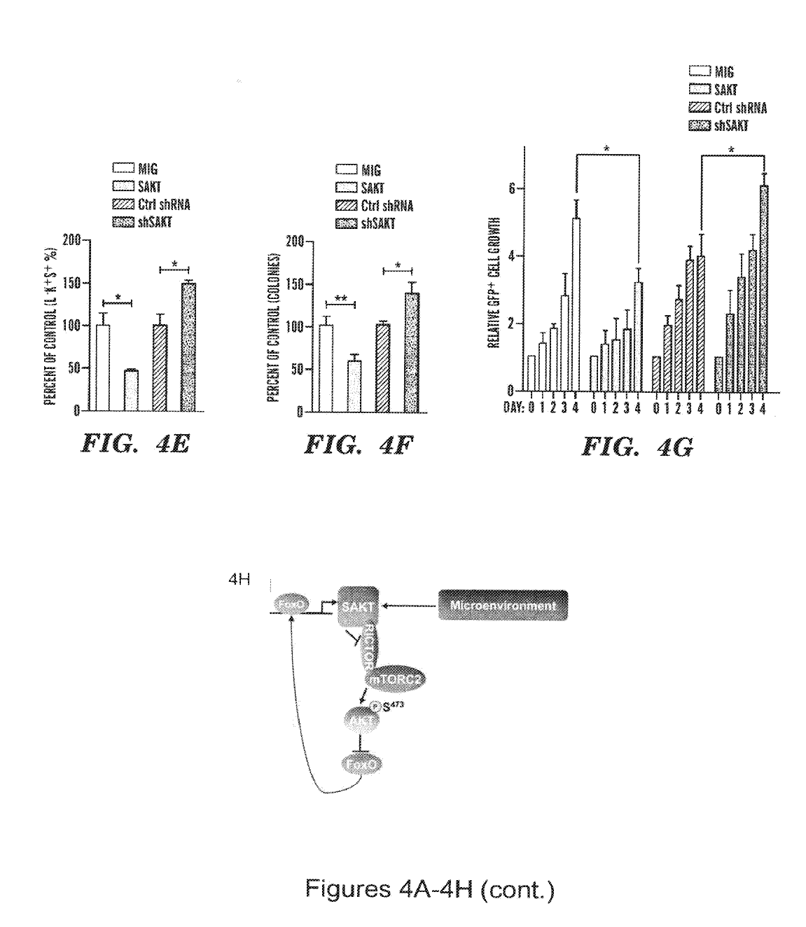

In mouse models, constitutive activation of AKT, either by enforced expression or genetic ablation of Pten, reduces the self-renewal properties of hematopoietic stem and progenitor cells (HSPCs) leading to their depletion (13-15). Furthermore, conditional compound deletion of FoxO1, FoxO3 and FoxO4 causes HSPC exhaustion and reduces hematopoietic lineage reconstitution in vivo (16, 17). These studies prompted the evaluation the biological consequences of altering SAKT expression in the murine hematopoietic system. To this end, BM cells from C57BL/6 5-fluorouracil (5-FU)—injected donors were transduced with cells overexpressing or shRNA-depleted for SAKT, and then performed bone marrow transplantation (BMT) assays (FIG. 4A). Peripheral blood analyses revealed reduced hematopoietic reconstitution of SAKT expressing donor cells (GFP⁺) in recipient mice compared to control donor cells (FIG. 4B). Analysis of the donor-derived BM compartment of recipients at 20 weeks after transplantation revealed that the frequency of total mononuclear BM cells (MNBCs) (FIG. 4C), Lin⁻Sca1⁺c-Kit⁺ (L⁻S⁺K⁺) cells (FIG. 4E), L⁻S⁺K⁺CD150⁺CD48⁻ (data not shown) and mature Mac1⁺ myeloid cells (FIG. 4D) were reduced upon enforcing SAKT expression. In vitro methylcellulose assays showed that enforced SAKT expression significantly reduced colony formation compared to control cells (FIG. 4F). Additionally, BM cells expressing SAKT had markedly reduced proliferation compared to control-infected cells (FIG. 4G). Furthermore, shRNA-mediated depletion of SAKT significantly increased hematopoietic reconstitution and cellular growth (FIGS. 4C and 4D and 4G). These data demonstrate the ability of SAKT modulation to alter the function of hematopoietic stem and progenitor cells in vitro and in vivo.

Of note, the hematopoietic modifications seen with SAKT manipulation are different than those observed with Rictor deletion. Previous work has shown that Rictor deletion does not affect HSPC in vivo (18). In addition, the knock-down phenotype presented here is distinct from the Pten^{-/-} phenotype (13-15). Therefore, SAKT is likely to have additional targets beyond that of Rictor and AKT which participate in the distinctive hematopoietic changes observed herein and perhaps in the alteration in phospho-S6^{Ser235/236} not seen with Rictor depletion in MEF cell lines studied by others (11). Testing for candidate additional pathways altered by SAKT indicated that STAT3 phosphorylation is also diminished by SAKT (FIG. 1A and FIG. 8) and STAT3 is a known modulator of hematopoietic stem and progenitor cells. Others have shown that dominant negative STAT3 expression leads to diminished hematopoietic reconstituting capacity (19, 20), an effect observed with SAKT herein. No evidence of SAKT interaction with gp130 was found (FIG. 8), a common, but not exclusive receptor mediating STAT3 phosphorylation. SAKT therefore can downmodulate signaling of pathways in addition to AKT.

The experiments described herein identify a previously undefined membrane receptor, termed SAKT, that negatively regulates AKT signaling via modulating the activity of the Rictor/mTORC2 complex (FIG. 4H). It joins the small number of Rictor/mTORC2 interactors including mSin1, a scaffold protein regulating Rictor/mTORC2 assembly (21, 22), Protor1 that increases mTORC2-mediated SGK1 activation (23-25) and ribosome components, an interaction important for cytokine signaling (26). As such it can alter the activity of one of the critical growth regulatory pathways in mammalian cells and a pathway that is frequently implicated in the disordered proliferation, differentiation and metabolism of human cancer. Its cell surface localization indicates that it may be a means by which exogenous cues can dampen the AKT activation associated with multiple cytokines and provides methods and compositions, as described elsewhere herein for pharmacologically AKT modulation. Without wishing to be bound by theory, this molecule is implicated in the etiology of the micro-environmentally induced AML seen in the Dicer deleted mouse model possibly through inducing the differentiation blockade of low pAKT/high FOXO found in ˜40% of human AML (8).

Materials and Methods

Mice and Animal Procedures. All mice were kept in a specific pathogen-free facility at Massachusetts General Hospital. All mice studies and breeding were carried out under the approval of Institutional Animal Care and Use Committee of Massachusetts General Hospital. FoxO1/3/4floxed; Mx1-Cre (8,16, 17) and Rictorfloxed; Mx1-Cre (25) mice were generated previously. For Bone Marrow Transplantation (BMT) assay, wild-type mice (CD45.2) were administered 150 mg/kg 5-flurouracil (5-FU, Sigma). Recovered bone-marrow (BM) cells was stimulated overnight in RPM/10% FBS with murine IL-3, IL-6, and stem cell factor. BM cells were then transduced twice with recombinant SAKT-expressing retroviruses or lentiviruses expressing shRNA against mouse SAKT, respectively, and transplanted into lethally irradiated recipient mice (CD45.1). Beginning four weeks after transplantation and continuing for 16 weeks, blood from the tail veins of recipient mice, was subjected to ammonium-chloride potassium (ACK) red cell lysis and quantities donor cell engraftment.

Materials. The cDNA of SAKT was cloned into the MSCV-IRES-GFP (MIG) to generate retrovirus expression vector. FLAG expression vectors for SAKT full length and ΔC mutant were generated by restriction enzyme digestion with BamHI-XhoI and PCR-based mutagenesis. The pLKO-shSAKT lentiviral vector (TRC number: TRCN0000176164) was purchased from Open Biosystems. For pLKO-GFP-shSAKT, the GFP marker was replaced by a puromycin resistance cassette subcloned into the BamHI and KpnI sites of pLKO. For luciferase reporter constructs, PCR-amplified murine SAKT promoter fragment from 129/Sv mouse tail genomic DNA was inserted into the XhoI and HindIII sites of pGL3-Basic (Promega). Antibodies to panAKT (#4691), phospho-AKTS473 (#4060), phospho-AKTT308 (#2965), PTEN (#9188), FoxO3 (#2497), phospho-FoxO3S253 (#9466), phospho-FoxO3T32 (#9464), mTOR (#2983), phospho-mTORS2448 (#5536), Rictor (#2114), S6 ribosomal protein (#2217), phospho-S6 ribosomal proteinS235/236 (#2211), 4EBP1 (#9644), phospho-4EBP1T37/46 (#2855), PRAS40 (#2691), phospho-PRAS40T246 (#2997), Raptor (#2280), GPL (#3274), ERK1/2 (#9102), STAT3 (#4904), phospho-STAT3Y705 (#9145), PDK1 (#3062), PP2A (#2041), GP130 (#3732), JAK1 (#3344), JAK2 (#3230), p85 (#4257), p110α (#4249), p110 β (#3011), p110γ (#5405) and β-actin (#4970) were from Cell Signaling Technology. Antibodies to SAKT (C14ORF37), PKCα, and phospho-PKCαS657, phospho-ERK1/2T202/204 were from Santa Cruz Biotechnology and FLAG M2 antibody was from Sigma.

Cell culture, Virus production, Transfection, and Stimulation. The retrovirus (for SAKT, FoxO3 WT and mutant, and myr-AKT) and lentivirus (for shSAKT) expression constructs were transfected using polyethylenimine reagent (Polysciences) into 293FT or 293TL cells, respectively. Media containing the recombinant retrovirus was collected for transduction at 48 hours post transfection, filtered through a 0.45 μm filter, and concentrated with PEG-it Virus Concentration Solution (System Biosciences) overnight at 4° C. (26). Viruses were precipitated next day and resuspended with PBS. For luciferase reporter assay, 293T cells (3×105/well of a 6-well plate) were transfected with 1 μg FoxO3 expression plasmid (FLAG-FoxO3), 200 ng pGL3-mSAKT promoter constructs, and 25 ng pRL-CMV by employing polyethylenimine (Polysciences).

Forty-eight hours later, cells were harvested, and luciferase activity was measured using the Dual-Luciferase reporter assay system (Promega) according to the manufacturer's instructions. Firefly luciferase values were divided by Renilla luciferase values to calculate transfection efficiency. Primers used for the construct of the SAKT promoter were were as follow: FoxO3 SAKT-F, 5′-AAT TCT CGA GAG TCA AAG AGT AGC TGT TCA TGA A (SEQ ID NO: 15) and FoxO3 SAKT-R, 5′-AAT TAA GCT TCT AAA AGC AAA ATG AGA GAG AGG A (SEQ ID NO: 16). Cell proliferation of GFP+ BM cells expressing SAKT or shRNA-mediated depletion of SAKT BM cells were followed by cell counting of samples in triplicate using a cellometer (Nexcelom Bioscience). For serum and insulin stimulation experiments, BM cells expressing SAKT or shRNA-mediated depletion of SAKT BM cells were deprived of serum for 3 hr, then FBS or insulin was add back at 10% serum or 1 μg/mL insulin concentration, respectively, for 30 min prior to lysis (11, 20).

Protein Analyses. For immunoblotting, cells were lysed in RIPA buffer (Cell Signaling Technology) supplemented with HALT™ protease and phosphatase inhibitor cocktails (Thermo Scientific). Western blot analysis was carried out according to standard methods (8). Equal amounts of total protein from lysates were subjected to SDS-PAGE, transferred to PVDF membrane (Invitrogen), and membranes were probed by overnight incubation with appropriate primary antibodies. Bound antibodies were visualized with HRP-conjugated secondary antibodies and ECL detection reagent (GE Healthcare) and quantification of protein bands (Multigauge V3.0, Fujifilm). For standard immunoprecipitation experiments, proteins were extracted by incubation on ice in IP Lysis Buffer and all protein extracts were pre-cleared with Protein-A/G agarose (Santa Cruz Biotechnology) and incubated with Protein A/G agarose bound with antibodies against FLAG M2 (Sigma), Rictor (#9476, Cell Signaling Technology), and SAKT (Santa Cruz Biotechnology) or control IgG from mouse, and rabbit at 4° C. Immunoprecipitates were washed four times with lysis buffer and boiled for 10 minutes in 1× LDS sample buffer (Invitrogen). The eluted proteins were immunoblotted with the antibodies indicated above. Chromatin Immunoprecipitation (ChIP) experiments were performed using a Chromatin Immunoprecipitation (ChIP) Assay Kit (Millipore) in accordance with the manufacturer's instructions (27). Briefly, SAKTexpressing NIH3T3 and BM cells were collected, and crosslinked with 1% formaldehyde. Cells were lysed, and chromatin was fragmented to 500 bp by sonication. The chromatin slurry was incubated with FLAG M2 (Sigma) or FoxO3 (#2497, Cell Signaling Technology) antibodies, control IgG from mouse, and rabbit, respectively, was used as a control. The immunoprecipitated DNA fragments were recovered and subjected to qPCR using the primers. Primer sequences were as follow: FoxO3 ChIP-F, 5′-ACA CGA AGC AAT GTT TTG TTT TA (SEQ ID NO: 17) and FoxO3 ChIP-R, 5′-AAG GAA GTC TCC CCT TCA CC (SEQ ID NO: 18); Albumin ChIP-F, 5′-CTC CAG ATG GCA AAC ATA CG (SEQ ID NO: 19) and Albumin ChIP-R, 5′-TCT GTG TGC AGA AAG ACT CG (SEQ ID NO: 20).

Quantitative (real-time) reverse-transcriptase PCR (qRT-PCR). Reverse transcription and quantitative PCR were performed as previously described (27, 28). Briefly, one microgram of total RNA was extracted from BM cells using RNeasy™ Plus Mini Kit (Qiagen). cDNA was made with iScript™ cDNA Synthesis Kit (Invitrogen). Quantitative RT-PCR was performed with SYBR Green™ Mix (Applied Biosystem) and a StepOne™ Real-Time PCR System instrument (Applied Biosystem), and data were normalized by the abundance of Gapdh mRNA. The normalized Ct values were measured by using the 2(-ΔCt) calculation method. Primer sequences were as follow: Gapdh F, 5′-GCA CAG TCA AGG CCG AGA AT (SEQ ID NO: 21) and Gapdh R, 5′-GCC TTC TCC ATG GTG GTG AA (SEQ ID NO: 22); SAKTF, AAC TCC TTC AAC CTG TTT TTC CC (SEQ ID NO: 23) and SAKTR, TGC AGC ATG TAC TTT GGA GCA (SEQ ID NO: 24); Rictor#1F, GCT GCG CTA TCT CAT CCA AGA (SEQ ID NO: 25) and Rictor#1R, GGG TTC TGA AGT GCT AGT TCA C (SEQ ID NO: 26); Rictor#2F, ACA GTT GGA AAA GTG GCA CAA (SEQ ID NO: 27) and Rictor#2R, GCG ACG AAC GTA GTT ATC ACC A (SEQ ID NO: 28).

Immunofluorescence assay. Immunofluorescence assays were performed as previously described (8). BM cells were plated on coated slides via cytospin (4min at 450 rpm), and fixed with 1% paraformaldehyde for 10 minutes. After washing, cells were permeabilized with methanol and blocked with 5% BSA. Slides were stained with anti-Rictor (Abcam) and anti-SAKT (Santa Cruz Biotechnology) antibodies at 4° C. overnight. After washing, slides were incubated with secondary antibodies conjugated with AlexaFluor 488 or 594 (Invitrogen), respectively, together with DAPI.

Flow Cytometry and Antibodies. BM cells and leukocytes were harvested and subjected to red cell lysis (8, 28). Fresh cells were stained with the following antibodies: cKit-PE, and -APC, Scal-APC, and -APC-Cy7, CD45.2-APC, Macl-APC, B220-PE, CD3-PE-Cy5, and lineage cocktail comprised of antibodies targeting CD3, CD4, CD8, CD19, B220, Gr1, Mac1, Ter119, and IL7Ra (BD Biosciences). Stained cells were analyzed with an LSRII, and FACSCalibur™ flow cytometer. Cell sorting was performed with a FACSAriaII™ instrument (Becton Dickinson). Data acquisition and analysis were performed with Cell Quest Pro™ or Diva™ software (BD Biosciences) and with FlowJo™ software (Tree Star), respectively.

Colony Formation Assays. For assessing hematopoietic progenitor cell activity, BM was harvested, subjected to redcell lysis, and nucleated BM cells were counted and plated in methylcellulose medium (M3434, STEMCELL Technologies). The colony number is counted 7 days after plating.

Statistical Analysis. In vitro and in vivo data were analyzed with an unpaired t test (GraphPad Prism™ (GraphPad Software Inc.) and SigmaPlot™ 10.0 software (SPSS Inc.)). Values of p<0.05 were considered statistically significant (*p<0.05; **p<0.01).

### REFERENCES AND NOTES

1. D. D. Sarbassov, S. M. Ali, D. M. Sabatini, Curr. Opin. Cell. Biol. 17, 596 (2005).
2. S. Wullschleger, R. Loewith, M. N. Hall, Cell 124, 471 (2006).
3. M. Laplante, D. M. Sabatini, J. Cell. Sci. 122, 3589 (2009).
4. R. Zoncu, A. Efeyan, D. M. Sabatini, Nat. Rev. Mol. Cell Bio. 12, 21 (2010).
5. D. A. Guertin, D. M. Sabatini, Trends. Mol. Med. 11, 353 (2005).
6. D. A. Guertin, D. M. Sabatini, Cancer Cell 12, 9 (2007).
7. M. H. G. P. Raaijmakers et al., Nature 464, 852 (2010).
8. S. M. Sykes et al., Cell 146, 697 (2011).
9. A. Brunet et al., Cell 96, 857 (1999).
10. E. L. Greer, A. Brunet, Oncogene 24, 7410 (2005).
11. D. A. Guertin et al., Dev. Cell 11, 859 (2006).
12. A. D. Kohn, S. A. Summers, M. J. Birnbaum, R. A. Roth, J. Biol. Chem. 271, 31372 (1996).
13. M. G. Kharas et al., Blood 115, 1406 (2010).
14. Ö. H. Yilmaz et al., Nature 441, 475 (2006).
15. J. Zhang et al., Nature 441, 518 (2006).
16. J. H. Paik et al., Cell 128, 309 (2007).
17. Z. Tothova et al., Cell 128, 325 (2007).
18. D. Kalaitzidis et al., Cell Stem Cell 11, 429 (2012).
19. I. H. Oh, C. J. Eaves, Oncogene 21, 4778 (2002).
20. Y. Chung et al., Blood 108, 1208 (2006).
21. M. A. Frias et al., Curr. Biol 16, 1865 (2006).
22. E. Jacinto et al., Cell 127, 125 (2006).
23. L. R. Pearce et al., Biochem. J. 405, 513 (2007).
24. K. Thedieck et al., PLoS ONE 2, e1217 (2007).
25. L. R. Pearce, E. M. Sommer, K. Sakamoto, S. Wullschleger, D. R. Alessi, Biochem. J. 436, 169 (2011).
26. V. Zinzalla, D. Stracka, W. Oppliger, M. N. Hall, Cell 144, 757 (2011).
27. C. Shiota, J. T. Woo, J. Lindner, K. D. Shelton, M. A. Magnuson, Dev. Cell. 11, 583 (2006).
28. N. Sun et al., Proc. Natl Acad. Sci. USA 106, 15720 (2009).
29. D. Lee et al., Cell Stem Cell 2, 497 (2008).
30. D. Lee, T. Kim, D. S. Lim, Stem Cells 29, 539 (2011).

What is claimed herein is:

1. A method of treating a subject having cancer, the method comprising:
determining, in a cancer cell sample obtained from the subject, the level of SAKT signaling activity; and
administering a treatment to the subject;
wherein a subject with a decreased level of SAKT signaling activity is administered a treatment comprising a therapeutically effective amount of an agonist of SAKT signaling; and
wherein a subject with an increased level of SAKT signaling activity, as compared to a reference level, is administered a treatment comprising a therapeutically effective amount of an inhibitor of SAKT signaling.

2. The method of claim 1, wherein increased SAKT signaling activity is determined by detecting a decreased level of AKT expression products and, optionally, an increased level of FOXO expression product.

3. The method of claim 1, wherein decreased SAKT signaling activity is determined by detecting an increased level of AKT expression products.

4. The method of claim 2, wherein the AKT expression products comprise phosphorylated AKT expression products.

5. The method of claim 1, wherein increased SAKT signaling activity is determined by detecting a marker selected from the group consisting of:
decreased levels of RICTOR; decreased levels of FOXO phosphorylation; decreased levels of FOXO3 phosphorylated at S253 and/or T32; decreased levels of mTOR phosphorylated at S2448; decreased levels of S6 phosphorylated at S235 and/or S236, decreased levels of 4EBP1 phosphorylated at T37 and/or T46; decreased levels of PRAS40 phosphorylated at T246; decreased levels of phosphorylated STAT3; decreased levels of phosphorylated SGK; decreased levels of SGK phosphorylated at S422; decreased levels of phosphorylated PKCα; decreased levels of PKCα phosphorylated at S638; and decreased levels of AKT phosphorylated at S473.

6. The method of claim 1, wherein decreased SAKT signaling activity is determined by detecting a marker selected from the group consisting of:
increased levels of RICTOR; increased levels of FOXO phosphorylation; increased levels of FOXO3 phosphorylated at S253 and/or T32; increased levels of mTOR phosphorylated at S2448; increased levels of S6 phosphorylated at S235 and/or S236; increased levels of 4EBP1 phosphorylated at T37 and/or T46; increased levels of PRAS40 phosphorylated at T246; increased levels of phosphorylated STAT3; increased levels of phosphorylated SGK; increased level of SGK phosphorylated at S422; increased levels of phosphorylated PKCα; increased levels of PKCα phosphorylated at S638; and increased levels of AKT phosphorylated at S473.

7. The method of claim 1, wherein the agonist of SAKT signaling is selected from the group consisting of:
an agonist of SAKT; an inhibitor of RICTOR; an inhibitor of mTORC2; and an inhibitor of RICTOR-mTORC2 interaction.

8. The method of claim 7, wherein the agonist of SAKT is selected from the group consisting of:
an antibody reagent agonist; and a nucleic acid encoding SAKT.

9. The method of claim 1, wherein the inhibitor of SAKT signaling is selected from the group consisting of:
an inhibitor of SAKT; an agonist of RICTOR; an agonist of mTORC2; and an agonist of RICTOR-mTORC2 interaction.

10. The method of claim 9, wherein the inhibitor of SAKT is selected from the group consisting of:
an inhibitory nucleic acid molecule; and an antibody reagent.

11. The method of claim 1, wherein the cell is selected from the group consisting of:
a hematopoietic cancer cell and an epithelial cancer cell.

12. The method of claim 11, wherein the epithelial cancer cell is selected from the group consisting of:
carcinoma; adenocarcinoma; basal cell carcinoma; squamous cell carcinoma; large cell carcinoma; small cell carcinoma; colorectal adenocarcinoma; lung cancer; breast cancer; prostate cancer; colon cancer; rectal cancer; pancreatic cancer; kidney cancer; ovarian cancer; stomach cancer; intestinal cancer; oral cancer; esophageal cancer; lip cancer; bladder cancer; cervical cancer; skin cancer; hepatocellular carcinoma; and renal cell carcinoma.

13. An assay comprising:
(a) contacting a cancer cell sample obtained from a subject with a detectable anti-AKT antibody reagent; and
(b) detecting the presence or intensity of a detectable signal;
wherein an increase in the level of AKT polypeptide, indicated by the level of the detectable signal, relative to a reference level indicates the subject is in need of treatment with an agonist of SAKT signaling activity; and
wherein a decrease in the level of AKT polypeptide, indicated by the level of the detectable signal, relative to a reference level indicates the subject is in need of treatment with an inhibitor of SAKT signaling activity.

14. The assay of claim 13, further comprising contacting the cancer cell sample with a detectable anti-Foxo antibody reagent

15. The assay of claim 13, wherein the anti-AKT antibody reagent is specific for AKT polyeptide phosphorylated at S473.

16. The assay of claim 13, wherein the agonist of SAKT signaling is selected from the group consisting of:
an agonist of SAKT; an inhibitor of RICTOR; an inhibitor of mTORC2; and an inhibitor of RICTOR-mTORC2 interaction.

17. A method of suppressing AKT activity in a cell, comprising contacting the cell with administering an agonist of SAKT activity or expression.

18. The method of claim 17, wherein the agonist of SAKT activity is selected from the group consisting of:
an agonist of SAKT; an inhibitor of RICTOR; an inhibitor of mTORC2; and an inhibitor of RICTOR-mTORC2 interaction.

19. The method of claim 17, wherein the agonist of SAKT is selected from the group consisting of:
an antibody reagent agonist; and a nucleic acid encoding SAKT.


SEQUENCE LISTING (28 sequences)

SEQ ID NO: 1 - protein, 480 aa, Homo sapiens [sequence omitted]

SEQ ID NO: 2 - DNA, 3008 bp, Homo sapiens
taattatgggtctgtaaccaccctggactgggtgctcctcactgacggacttgtctgaacctctctttgtctccagcgcccagcactgggcctggcaaaacctgagacgcccggtacatgttggccaaatgaatgaaccagattcagaccggcaggggcgctgtggtttaggaggggcctggggtttctcccaggaggtttttgggcttgcgctggagggctctggactcccgtttgcgccagtggcctgcatcctggtcctgtcttcctcatgtttgaatttctttgctttcctagtctggggagcagggaggagccctgtgccctgtcccaggatccatgggtaggaacaccatggacagggagagcaaacggggccatctgtcaccaggggcttagggaaggccgagccagcctgggtcaaagaagtcaaaggggctgcctggaggaggcagcctgtcagctggtgcatcagaggctgtggccaggccagctgggctcggggagcgccagcctgagaggagcgcgtgagcgtcgcgggagcctcgggcaccatgagcgacgtggctattgtgaaggagggttggctgcacaaacgaggggagtacatcaagacctggcggccacgctacttcctcctcaagaatgatggcaccttcattggctacaaggagcggccgcaggatgtggaccaacgtgaggctcccctcaacaacttctctgtggcgcagtgccagctgatgaagacggagcggccccggcccaacaccttcatcatccgctgcctgcagtggaccactgtcatcgaacgcaccttccatgtggagactcctgaggagcgggaggagtggacaaccgccatccagactgtggctgacggcctcaagaagcaggaggaggaggagatggacttccggtcgggctcacccagtgacaactcaggggctgaagagatggaggtgtccctggccaagcccaagcaccgcgtgaccatgaacgagtttgagtacctgaagctgctgggcaagggcactttcggcaaggtgatcctggtgaaggagaaggccacaggccgctactacgccatgaagatcctcaagaaggaagtcatcgtggccaaggacgaggtggcccacacactcaccgagaaccgcgtcctgcagaactccaggcaccccttcctcacagccctgaagtactctttccagacccacgaccgcctctgctttgtcatggagtacgccaacgggggcgagctgttcttccacctgtcccgggagcgtgtgttctccgaggaccgggcccgcttctatggcgctgagattgtgtcagccctggactacctgcactcggagaagaacgtggtgtaccgggacctcaagctggagaacctcatgctggacaaggacgggcacattaagatcacagacttcgggctgtgcaaggaggggatcaaggacggtgccaccatgaagaccttttgcggcacacctgagtacctggcccccgaggtgctggaggacaatgactacggccgtgcagtggactggtgggggctgggcgtggtcatgtacgagatgatgtgcggtcgcctgcccttctacaaccaggaccatgagaagctttttgagctcatcctcatggaggagatccgcttcccgcgcacgcttggtcccgaggccaagtccttgctttcagggctgctcaagaaggaccccaagcagaggcttggcgggggctccgaggacgccaaggagatcatgcagcatcgcttctttgccggtatcgtgtggcagcacgtgtacgagaagaagctcagcccacccttcaagccccaggtcacgtcggagactgacaccaggtattttgatgaggagttcacggcccagatgatcaccatcacaccacctgaccaagatgacagcatggagtgtgtggacagcgagcgcaggccccacttcccccagttctcctactcggccagcggcacggcctgaggcggcggtggactgcgctggacgatagcttggagggatggagaggcggcctcgtgccatgatctgtatttaatggtttttatttctcgggtgcatttgagagaagccacgctgtcctctcgagcccagatggaaagacgtttttgtgctgtgggcagcaccctcccccgcagcggggtagggaagaaaactatcctgcgggttttaatttatttcatccagtttgttctccgggtgtggcctcagccctcagaacaatccgattcacgtagggaaatgttaaggacttctgcagctatgcgcaatgtggcattggggggccgggcaggtcctgcccatgtgtcccctcactctgtcagccagccgccctgggctgtctgtcaccagctatctgtcatctctctggggccctgggcctcagttcaacctggtggcaccagatgcaacctcactatggtatgctggccagcaccctctcctgggggtggcaggcacacagcagccccccagcactaaggccgtgtctctgaggacgtcatcggaggctgggcccctgggatgggaccagggatgggggatgggccagggtttacccagtgggacagaggagcaaggtttaaatttgttattgtgtattatgttgttcaaatgcattttgggggtttttaatctttgtgacaggaaagccctcccccttccccttctgtgtcacagttcttggtgactgtcccaccgggagcctccccctcagatgatctctccacggtagcacttgaccttttcgacgcttaacctttccgctgtcgccccaggccctccctgactccctgtgggggtggccatccctgggcccctccacgcctcctggccagacgctgccgctgccgctgcaccacggcgtttttttacaacattcaactttagtatttttactattataatataatatggaaccttccctccaaattcttcaataaaagttgcttttcaaaaaaaaaaaaaaaaaaaaaa

SEQ ID NO: 3 - protein, 1708 aa, Homo sapiens
MAAIGRGRSLKNLRVRGRNDSGEENVPLDLTREPSDNLREILQNVARLQGVSNMRKLGHLNNFTKLLCDIGHSEEKLGFHYEDIIICLRLALLNEAKEVRAAGLRALRYLIQDSSILQKVLKLKVDYLIARCIDIQQSNEVERTQALRLVRKMITVNASLFPSSVTNSLIAVGNDGLQERDRMVRACIAIICELALQNPEVVALRGGLNTILKNVIDCQLSRINEALITTILHLLNHPKTRQYVRADVELERILAPYTDFHYRHSPDTAEGQLKEDREARFLASKMGIIATFRSWAGIINLCKPGNSGIQSLIGVLCIPNMEIRRGLLEVLYDIFRLPLPVVTEEFIEALLSVDPGRFQDSWRLSDGFVAAEAKTILPHRARSRPDLMDNYLALILSAFIRNGLLEGLVEVITNSDDHISVRATILLGELLHMANTILPHSHSHHLHCLPTLMNMAASFDIPKEKRLRASAALNCLKRFHEMKKRGPKPYSLHLDHIIQKAIATHQKRDQYLRVQKDIFILKDTEEALLINLRDSQVLQHKENLEWNWNLIGTILKWPNVNLRNYKDEQLHRFVRRLLYFYKPSSKLYANLDLDFAKAKQLTVVGCQFTEFLLESEEDGQGYLEDLVKDIVQWLNASSGMKPERSLQNNGLLTTLSQHYFLFIGTLSCHPHGVKMLEKCSVFQCLLNLCSLKNQDHLLKLTVSSLDYSRDGLARVILSKILTAATDACRLYATKHLRVLLRANVEFFNNWGIELLVTQLHDKNKTISSEALDILDEACEDKANLHALIQMKPALSHLGDKGLLLLLRFLSIPKGFSYLNERGYVAKQLEKWHREYNSKYVDLIEEQLNEALTTYRKPVDGDNYVRRSNQRLQRPHVYLPIHLYGQLVHHKTGCHLLEVQNIITELCRNVRTPDLDKWEEIKKLKASLWALGNIGSSNWGLNLLQEENVIPDILKLAKQCEVLSIRGTCVYVLGLIAKTKQGCDILKCHNWDAVRHSRKHLWPVVPDDVEQLCNELSSIPSTLSLNSESTSSRHNSESESVPSSMFILEDDRFGSSSTSTFFLDINEDTEPTFYDRSGPIKDKNSFPFFASSKLVKNRILNSLTLPNKKHRSSSDPKGGKLSSESKTSNRRIRTLTEPSVDFNHSDDFTPISTVQKTLQLETSFMGNKHIEDTGSTPSIGENDLKFTKNFGTENHRENTSRERLVVESSTSSHMKIRSQSFNTDTTTSGISSMSSSPSRETVGVDATTMDTDCGSMSTVVSTKTIKTSHYLTPQSNHLSLSKSNSVSLVPPGSSHTLPRRAQSLKAPSIATIKSLADCNFSYTSSRDAFGYATLKRLQQQRMHPSLSHSEALASPAKDVLFTDTITMKANSFESRLTPSRFMKALSYASLDKEDLLSPINQNTLQRSSSVRSMVSSATYGGSDDYIGLALPVDINDIFQVKDIPYFQTKNIPPHDDRGARAFAHDAGGLPSGTGGLVKNSFHLLRQQMSLTEIMNSIHSDASLFLESTEDTGLQEHTDDNCLYCVCIEILGFQPSNQLSAICSHSDFQDIPYSDWCEQTIHNPLEVVPSKFSGISGCSDGVSQEGSASSTKSTELLLGVKTIPDDTPMCRILLRKEVLRLVINLSSSVSTKCHETGLLTIKEKYPQTFDDICLYSEVSHLLSHCTFRLPCRRFIQELFQDVQFLQMHEEAEAVLATPPKQPIVDTSAES

SEQ ID NO: 4 - DNA, 9535 bp, Homo sapiens
gggttgtgactgaaacccgtcaatatggcggcgatcggccgcggccgctctctgaagaacctccgagtacgagggcggaatgacagcggcgaggagaacgtcccgctggatctgacccgagaaccttctgataacttaagagagattctccaaaatgtggccagattgcagggagtatcaaatatgagaaagctaggccatctgaataactttactaagcttctttgtgatattggccacagtgaagaaaaactgggctttcactatgaggatatcataatttgtttgcggttagctttattaaatgaagcaaaagaagtgcgagcagcagggctacgagcgcttcgatatctcatccaagactccagtattctccagaaggtgctaaaattgaaagtggactatttaatagctaggtgcattgacatacaacagagcaacgaggtagagaggacacaagcacttcgattagtcagaaagatgattactgtgaatgcttccttgtttcctagttctgtgaccaactcattaattgcagttggaaatgatggacttcaagaaagagacagaatggtccgagcatgcattgccattatctgtgaactagcacttcagaatccagaggtggtggcccttcgaggtggactaaacaccatcttgaaaaatgtgatcgattgccaattaagtcgaataaatgaggccctaattactacaattttgcaccttcttaatcatccaaagactcgacagtatgtgcgagctgatgtagaattagagagaattttagcaccctatactgattttcactacagacatagtccagatacagctgaaggacagctcaaagaagacagagaagcacgatttctagccagtaaaatgggaatcatagcaacattccgatcatgggcaggtattattaatttatgtaaacctggaaattctgggatccagtctctaataggagtactttgcataccaaatatggaaataaggcgaggtctacttgaagtgctttatgatatatttcgtcttcctctacctgttgtgactgaggagttcatagaagcactactcagtgtagatccagggaggttccaagacagttggaggctttcagatggctttgtggcagctgaggcaaaaactattcttcctcatcgtgccagatccaggccagacctcatggataattatttggcactgatactctctgcatttattcgtaatggacttttagagggtctagttgaagtgataacaaacagtgatgatcatatctcagttagagctaccatccttttaggagagcttttacatatggcaaacacaattcttcctcattcacatagccatcatttacactgcttgccaaccctaatgaatatggctgcatcctttgatatccccaaggaaaagagactgcgagccagtgcagccttgaactgtttaaaacgcttccatgaaatgaagaaacgaggacctaagccttatagtcttcatttagaccacattattcagaaagcaattgcaacacaccagaaacgggatcagtatctccgagttcagaaagatatatttatccttaaggatacagaggaagctcttttaattaaccttagagatagccaagtccttcaacataaagagaatcttgaatggaattggaatcttatagggaccattcttaagtggccaaatgtaaatctaagaaactataaagatgaacagttacacaggtttgtacgaagactactttatttttacaagcccagcagtaaattatatgccaacctggatctggattttgccaaggccaaacagctcacggttgtaggttgccagtttacagaatttcttcttgaatctgaagaggatgggcaaggctacttagaagatctagtaaaggatattgttcagtggctcaatgcttcatctggaatgaaacccgaaagaagtcttcaaaataatggtttattgaccacccttagtcaacactactttttatttattggaacactttcttgccaccctcatggagttaaaatgctggaaaaatgcagtgtatttcagtgtctccttaatctttgctccttgaaaaaccaagatcacttgctaaaacttactgtttctagcttggactatagcagagatggattggctagagtcatcctttccaaaattttaactgcagctactgatgcctgcagactctatgcaacaaaacatttaagggtattattgagagctaatgttgaattctttaataattggggaattgagttgttagtgacccagctacatgataaaaacaaaacgatttcctctgaagctcttgatatcctcgatgaagcatgtgaagacaaggccaatcttcatgctctcattcagatgaaaccagcgttatcccaccttggagacaagggtttgcttctcctgctgagatttctctccattccaaaaggattttcctatctgaatgaaagaggttatgtagcaaaacaattggaaaagtggcacagggaatacaactccaaatatgttgacttgattgaggaacaactcaatgaagcacttactacttaccggaagcctgttgatggtgataactatgttcgtcggagtaaccaaagattacagcgtcctcacgtctacctgcctatacacctttatggacaactagtacaccataaaacaggctgccatttgttggaagtacagaatattattacagaactctgtcgtaatgttcgtacaccagatttggataagtgggaagaaattaaaaaactgaaagcatctctttgggccttgggaaatatcggctcatcaaattggggtctcaatttgctacaggaagaaaacgtgattccagatatactaaaacttgcaaaacagtgtgaagttctttccatcagagggacctgtgtatatgtacttgggctcatagctaaaaccaaacaaggctgtgatattctaaaatgtcacaactgggatgctgtgaggcatagtcgcaaacatctgtggccagtggttccagatgatgtggaacaactctgtaatgaactttcatctatcccaagcactctaagtttgaactcggagtcaaccagctctagacataatagtgaaagtgaatctgtgccatcgagtatgttcatattggaggatgaccggtttggcagcagctctactagtacatttttccttgatatcaatgaagatacagagccaacattttatgaccgatctggacccataaaggataaaaattcattccctttctttgcttctagtaaacttgtgaagaatcgtatcttaaattcgcttactttgcctaacaaaaaacatcgtagtagcagtgatccaaaaggagggaaattatcatctgaaagtaagacaagcaacaggcgaatcagaacacttacggagcccagtgttgattttaatcatagtgatgattttacacccatatccactgtacagaaaacattacaattagagacttcatttatggggaataagcacattgaagacactggtagtacaccaagcattggagaaaatgacttaaaattcaccaagaattttggtacagagaatcacagagaaaatacaagccgagagaggttagtagtagaaagttcaacgagctcacatatgaagatacgtagccaaagtttcaatacagacactacaacaagtggcataagttcaatgagctcaagtccttcacgagagacagtaggtgtagatgctacaactatggacacagactgtggaagcatgagtactgtggtaagtactaaaactattaagacaagccactatttgacgccacagtctaaccatctgtctctctccaaatcaaattcggtgtccctggtgcctccaggttcttctcatacgcttcctagaagagcacagtcccttaaagcaccctctattgctacaattaaaagtctagcagattgtaactttagttacacaagttctagagatgcttttggctatgctacactgaaaagactacagcaacaaagaatgcatccatccttatctcactctgaagctttggcatctccagcaaaagatgtgctatttactgataccatcaccatgaaggccaacagttttgagtccagattaacaccaagcaggttcatgaaagccttaagttatgcatcattagataaagaagatttattgagtcctattaatcaaaataccctgcaacgatcttcctcagtgcggtccatggtgtccagtgccacatatgggggttcagatgattacattggtcttgctctcccggtggatataaatgatatattccaggtaaaggatattccctattttcagacaaaaaacataccaccacatgatgatcgaggtgcaagagcatttgcccatgatgcaggaggtcttccatctggaactggaggtcttgtaaaaaattcttttcacttgctacgacagcagatgagtcttacggaaataatgaattcaatccattcagatgcctctctgtttttagaaagtacagaagacactggactacaggaacatacagatgataactgcctttattgtgtctgtattgaaattctgggtttccagcccagcaaccaactgagtgcaatatgtagtcattcagactttcaagatattccatattctgattggtgtgagcagactatccataatcctttagaagtggttccctctaagttttcggggatttctggatgcagtgatggggtgtctcaagaaggctcagctagcagcaccaaaagcacagaattgttactaggtgttaaaacaattccagatgatacaccaatgtgccgtatactccttcgcaaagaagttctaagattagtcattaatttgagtagttcagtttcaactaaatgtcatgagactgggcttttaacaattaaggagaagtatcctcaaacatttgatgacatatgcctttactctgaggtttcccatttgctgtcacactgcacattcagacttccgtgtcggaggttcatacaagaattatttcaagatgtacagtttctacaaatgcatgaagaagcagaggctgtgttggcaacaccaccaaagcaacctatagttgatacatctgctgaatcctgacctcatatttatgatggatatagatacatactatatatattcatatttgtggatttcctaaaagcctcagaaaatacgactgactaggcagcaaagacaggagtatcttctgtacactgttccgcagttactggtacatgaacagttggaactgctgactttcctaaccaaaacaacttccttctctcctttgttgagccttttgaggggttcatgattcattaccacagttttaagagtttcagttaccattgtatgcaagagccaagcactgaatacctacataggttttctattttctttcattttaaaagcataatgacagtggaacaataatgggatatgcagaagcacccttcacaagttatttctgaatgatttttagggtaaataatacagatgccttgtttgttaactaacttgtggaaagcaggaatcagtgtctctaaggctgcatcctattaccacaatggggtgtgctataactgctggtattagagagggaactttggccctttcacgtttttcttaatgtttgtaacactacttcagaggtttataacctcaaagcagaagaagagcctcaacaacccgggacttataagttatttttatgttactagacttgcataaagattcttgttttccaactcttcattttgttgcaatgtgttattacaggatatatgaaccaattaaggtttttcactacagttcttgaataaaatttaaaaatcattttttattttaattaaaaatatttcccatttatagaatgcatatatttgcaatggacttccactttcatcaactttccatctcatcgctttaaacaggaacttgaacaagcactgttagtttagacctaaaggataggaaagcattaaataatactttggatctcctgaggaaaagataagtttgcttgcaatttacacattccatggggaaagaagagccatatttccttaaaaaaaacattaataaagcttgttattgagaaaaattgtagtgaaaagccttaagtaccaaattttaaagcagcagtaacttaatttttatatcagtgtttttgttttgcacaaactaaatgcagtggtaggtgggtttatgagtatattaattgcctttatccatttgtgaagttaagttgatgagggcaaggtttttgtttgtttaatttgtatatgtctaaaggtatttggaactttttacaggaattaaacatatatgcaaatttgtatataaaaatagcatggccatcatttgaatgcttgtaaatgaaaggattatcttttttgagatctatatataaatagaaatagaaaatccagctggactgattaggattcttttttaattcatttgtgtataacatttttattacaattacacatcagttttgacacagtcatagcaacattaatattttcccatgatgcagatcctttttgtaatgggcttgttctttgagatctctgtaaagaaccctgtgaactagaaaacataactcacagagatacttttttaaaaaatttatttactggaactgaaagttccagttgggatgaagcatttcatctcacttcataacacctctttgactgcacttcagtgaattgttcttatgtgcactgtgtagcaacttacattataacaaagcagataagggctgtaagctgctgcttatgttgaaaagtggttcttcagattttctctcataaaatccagttgaagataaataatttttttatactttatcactgaacccaagtgtttatttaaatgtcaacagtacttctaagaacgttgcctgtcatcgtggtctttggtcttggataactaaactgcctttccagagaaccaaatgtcagagttactagaccaaatagtggttaaaacctccaaaggaagtaatgtaatcttattcataatgggattaacatattttagacattcattttaaacactacctcagttaatatagagtataaaaatctgtggtttaatccctcaaaagttaacagtaattttttttttgtcttacacacacacacaccccctcccccaccatcactatccctgtaccctcaccttggtcatctatcctgaaataaggcttagttagtattggcctgaatgttttgtgtttttttttttgttttttttttttactgttactttgaaaaatatgtatgtataccttatcatatctgcctatatcacttactttggggagatactcagagctttgtggttatcagtatactaaaaaaaaaaaaaagtctacgcttaaatttatagtgctatttggtttctccatgatttcactgacaggtctaatacattttctttgagtacttgtttgtaaaaagtagactttatggtgaaaaatacatgcagtgccaagtgattaacttaagtgtttaaaaatattaaattatagcagaagaggttaggaatgatatcagcagtaatagaaataattgagaaaatcatctataaataatagatattacagactatagaataccaaaataatgtcaatactgtagtttttaaagattttaggattaatcttagtccatataaatttgtactattggtaattattgaataattgggaggaatctgggcagttgtgctggttgtaaactatgaatttctaatcgtaaagtgaattgttatttctaattgaactttttttcaagaacagatttcagcctcacatactaagtaaatactgataaataaggaaattagaaatttagtattcataattaaatatgctctaaaatttcctatacttttatttcctgtttattcttaggtagattggaagggggaaacagtctgttctccctaattaaattttttctaataacgattagtagaatatggacattctatatgacagtgacattaaaagaggctctttggaagtatatacattattaacataatgtgtacaagtccttttgaaatgacaactttaatgggtttcagctcttttatctagagcttgagataattcaagctgagtttttcagggcatatcacaacggccaagtgttcagcagtgggatatcaatgcttatttacattttcctactgctatttatataaaatgttattccattcagaggatgccttttatccccacattaaagcacagatcattaagcaataaaaaccaaattgtctgtcattcaaattataactgcagttatttttgcatggtaagagtgaggtgctaattttgtgtgagatgaactttgtaaactactttgggaaatgttctttggaagtaaggttttttctcctttagtcttatgcttccacttttgtctcagattcacaatccattaaaacatggggaaaaaagaaaaggtaaaattgagagacttttgttagaggagctatttggaatgaaccaacatttcagattttccaaaatgtaagttaggaagtctccattgtctctgcattaacaaaatacactgttactatcttaatctcaagagtgtcattacagtgagaatctcatttaaaagcataccagtgaaattaatagcagtgcttatcaaagaacactgaaatctgtgagaatctttctaggagcattcttttcttcttttagttccaagttccagggtatttttcattcctagtaggtttatatgactcacagaatgtggacttttttcctgtttggagtatttttgtaatgtaagtatcggatagctgcaccacagcatgcataaattgcacattttgttttactttctttatagaatatttaatttcaaaaatataatttatgccaaaaaaagcatacctttcaattttgctacttggttgatttagcacaaaatgcaaagtcttggggcagagagggggagtgaaaaaaattttataggtaattgttacaaaaatacctgtcagaaaccctaaagctgcattgtaaaacaaatggtgtaaactagttttgaaaagtggtaaggaattgtgaaaaaaatctcagacttaatgctctctaaccacatgagtttcttcttttttatttagtaatacgctgctacatatttggaggttctggtgtttgtaggtcactgaacagacattgaaatctgatttatattgtataactgtaacatagaaagaaaaagtatttatattttttctgtaagaatatttcattgagttgtgtataatttaaataagatttgtccccaaatggttttgctcaccttgattttttttgttgtgattttcttgtttttgtataatgtgtatagtttatgtcaagggcattaaaagcctcctgaagcataatcttatcaaagggatacattgttaataaaatgtacttaaaattcttaaa

SEQ ID NO: 5 - protein, 2549 aa, Homo sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW

SEQ ID NO: 6 - DNA, 8733 bp, Homo sapiens
gctcccggcttagaggacagcggggaaggcgggcggtggggcagggggcctgaagcggcggtaccggtgctggcggcggcagctgaggccttggccgaagccgcgcgaacctcagggcaagatgcttggaaccggacctgccgccgccaccaccgctgccaccacatctagcaatgtgagcgtcctgcagcagtttgccagtggcctaaagagccggaatgaggaaaccagggccaaagccgccaaggagctccagcactatgtcaccatggaactccgagagatgagtcaagaggagtctactcgcttctatgaccaactgaaccatcacatttttgaattggtttccagctcagatgccaatgagaggaaaggtggcatcttggccatagctagcctcataggagtggaaggtgggaatgccacccgaattggcagatttgccaactatcttcggaacctcctcccctccaatgacccagttgtcatggaaatggcatccaaggccattggccgtcttgccatggcaggggacacttttaccgctgagtacgtggaatttgaggtgaagcgagccctggaatggctgggtgctgaccgcaatgagggccggagacatgcagctgtcctggttctccgtgagctggccatcagcgtccctaccttcttcttccagcaagtgcaacccttctttgacaacatttttgtggccgtgtgggaccccaaacaggccatccgtgagggagctgtagccgcccttcgtgcctgtctgattctcacaacccagcgtgagccgaaggagatgcagaagcctcagtggtacaggcacacatttgaagaagcagagaagggatttgatgagaccttggccaaagagaagggcatgaatcgggatgatcggatccatggagccttgttgatccttaacgagctggtccgaatcagcagcatggagggagagcgtctgagagaagaaatggaagaaatcacacagcagcagctggtacacgacaagtactgcaaagatctcatgggcttcggaacaaaacctcgtcacattacccccttcaccagtttccaggctgtacagccccagcagtcaaatgccttggtggggctgctggggtacagctctcaccaaggcctcatgggatttgggacctcccccagtccagctaagtccaccctggtggagagccggtgttgcagagacttgatggaggagaaatttgatcaggtgtgccagtgggtgctgaaatgcaggaatagcaagaactcgctgatccaaatgacaatccttaatttgttgccccgcttggctgcattccgaccttctgccttcacagatacccagtatctccaagataccatgaaccatgtcctaagctgtgtcaagaaggagaaggaacgtacagcggccttccaagccctggggctactttctgtggctgtgaggtctgagtttaaggtctatttgcctcgcgtgctggacatcatccgagcggccctgcccccaaaggacttcgcccataagaggcagaaggcaatgcaggtggatgccacagtcttcacttgcatcagcatgctggctcgagcaatggggccaggcatccagcaggatatcaaggagctgctggagcccatgctggcagtgggactaagccctgccctcactgcagtgctctacgacctgagccgtcagattccacagctaaagaaggacattcaagatgggctactgaaaatgctgtccctggtccttatgcacaaaccccttcgccacccaggcatgcccaagggcctggcccatcagctggcctctcctggcctcacgaccctccctgaggccagcgatgtgggcagcatcactcttgccctccgaacgcttggcagctttgaatttgaaggccactctctgacccaatttgttcgccactgtgcggatcatttcctgaacagtgagcacaaggagatccgcatggaggctgcccgcacctgctcccgcctgctcacaccctccatccacctcatcagtggccatgctcatgtggttagccagaccgcagtgcaagtggtggcagatgtgcttagcaaactgctcgtagttgggataacagatcctgaccctgacattcgctactgtgtcttggcgtccctggacgagcgctttgatgcacacctggcccaggcggagaacttgcaggccttgtttgtggctctgaatgaccaggtgtttgagatccgggagctggccatctgcactgtgggccgactcagtagcatgaaccctgcctttgtcatgcctttcctgcgcaagatgctcatccagattttgacagagttggagcacagtgggattggaagaatcaaagagcagagtgcccgcatgctggggcacctggtctccaatgccccccgactcatccgcccctacatggagcctattctgaaggcattaattttgaaactgaaagatccagaccctgatccaaacccaggtgtgatcaataatgtcctggcaacaataggagaattggcacaggttagtggcctggaaatgaggaaatgggttgatgaactttttattatcatcatggacatgctccaggattcctctttgttggccaaaaggcaggtggctctgtggaccctgggacagttggtggccagcactggctatgtagtagagccctacaggaagtaccctactttgcttgaggtgctactgaattttctgaagactgagcagaaccagggtacacgcagagaggccatccgtgtgttagggcttttaggggctttggatccttacaagcacaaagtgaacattggcatgatagaccagtcccgggatgcctctgctgtcagcctgtcagaatccaagtcaagtcaggattcctctgactatagcactagtgaaatgctggtcaacatgggaaacttgcctctggatgagttctacccagctgtgtccatggtggccctgatgcggatcttccgagaccagtcactctctcatcatcacaccatggttgtccaggccatcaccttcatcttcaagtccctgggactcaaatgtgtgcagttcctgccccaggtcatgcccacgttccttaacgtcattcgagtctgtgatggggccatccgggaatttttgttccagcagctgggaatgttggtgtcctttgtgaagagccacatcagaccttatatggatgaaatagtcaccctcatgagagaattctgggtcatgaacacctcaattcagagcacgatcattcttctcattgagcaaattgtggtagctcttgggggtgaatttaagctctacctgccccagctgatcccacacatgctgcgtgtcttcatgcatgacaacagcccaggccgcattgtctctatcaagttactggctgcaatccagctgtttggcgccaacctggatgactacctgcatttactgctgcctcctattgttaagttgtttgatgcccctgaagctccactgccatctcgaaaggcagcgctagagactgtggaccgcctgacggagtccctggatttcactgactatgcctcccggatcattcaccctattgttcgaacactggaccagagcccagaactgcgctccacagccatggacacgctgtcttcacttgtttttcagctggggaagaagtaccaaattttcattccaatggtgaataaagttctggtgcgacaccgaatcaatcatcagcgctatgatgtgctcatctgcagaattgtcaagggatacacacttgctgatgaagaggaggatcctttgatttaccagcatcggatgcttaggagtggccaaggggatgcattggctagtggaccagtggaaacaggacccatgaagaaactgcacgtcagcaccatcaacctccaaaaggcctggggcgctgccaggagggtctccaaagatgactggctggaatggctgagacggctgagcctggagctgctgaaggactcatcatcgccctccctgcgctcctgctgggccctggcacaggcctacaacccgatggccagggatctcttcaatgctgcatttgtgtcctgctggtctgaactgaatgaagatcaacaggatgagctcatcagaagcatcgagttggccctcacctcacaagacatcgctgaagtcacacagaccctcttaaacttggctgaattcatggaacacagtgacaagggccccctgccactgagagatgacaatggcattgttctgctgggtgagagagctgccaagtgccgagcatatgccaaagcactacactacaaagaactggagttccagaaaggccccacccctgccattctagaatctctcatcagcattaataataagctacagcagccggaggcagcggccggagtgttagaatatgccatgaaacactttggagagctggagatccaggctacctggtatgagaaactgcacgagtgggaggatgcccttgtggcctatgacaagaaaatggacaccaacaaggacgacccagagctgatgctgggccgcatgcgctgcctcgaggccttgggggaatggggtcaactccaccagcagtgctgtgaaaagtggaccctggttaatgatgagacccaagccaagatggcccggatggctgctgcagctgcatggggtttaggtcagtgggacagcatggaagaatacacctgtatgatccctcgggacacccatgatggggcattttatagagctgtgctggcactgcatcaggacctcttctccttggcacaacagtgcattgacaaggccagggacctgctggatgctgaattaactgcgatggcaggagagagttacagtcgggcatatggggccatggtttcttgccacatgctgtccgagctggaggaggttatccagtacaaacttgtccccgagcgacgagagatcatccgccagatctggtgggagagactgcagggctgccagcgtatcgtagaggactggcagaaaatccttatggtgcggtcccttgtggtcagccctcatgaagacatgagaacctggctcaagtatgcaagcctgtgcggcaagagtggcaggctggctcttgctcataaaactttagtgttgctcctgggagttgatccgtctcggcaacttgaccatcctctgccaacagttcaccctcaggtgacctatgcctacatgaaaaacatgtggaagagtgcccgcaagatcgatgccttccagcacatgcagcattttgtccagaccatgcagcaacaggcccagcatgccatcgctactgaggaccagcagcataagcaggaactgcacaagctcatggcccgatgcttcctgaaacttggagagtggcagctgaatctacagggcatcaatgagagcacaatccccaaagtgctgcagtactacagcgccgccacagagcacgaccgcagctggtacaaggcctggcatgcgtgggcagtgatgaacttcgaagctgtgctacactacaaacatcagaaccaagcccgcgatgagaagaagaaactgcgtcatgccagcggggccaacatcaccaacgccaccactgccgccaccacggccgccactgccaccaccactgccagcaccgagggcagcaacagtgagagcgaggccgagagcaccgagaacagccccaccccatcgccgctgcagaagaaggtcactgaggatctgtccaaaaccctcctgatgtacacggtgcctgccgtccagggcttcttccgttccatctccttgtcacgaggcaacaacctccaggatacactcagagttctcaccttatggtttgattatggtcactggccagatgtcaatgaggccttagtggagggggtgaaagccatccagattgatacctggctacaggttatacctcagctcattgcaagaattgatacgcccagacccttggtgggacgtctcattcaccagcttctcacagacattggtcggtaccacccccaggccctcatctacccactgacagtggcttctaagtctaccacgacagcccggcacaatgcagccaacaagattctgaagaacatgtgtgagcacagcaacaccctggtccagcaggccatgatggtgagcgaggagctgatccgagtggccatcctctggcatgagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagcagctgcctcagctcacatccttagagctgcaatatgtttccccaaaacttctgatgtgccgggaccttgaattggctgtgccaggaacatatgaccccaaccagccaatcattcgcattcagtccatagcaccgtctttgcaagtcatcacatccaagcagaggccccggaaattgacacttatgggcagcaacggacatgagtttgttttccttctaaaaggccatgaagatctgcgccaggatgagcgtgtgatgcagctcttcggcctggttaacacccttctggccaatgacccaacatctcttcggaaaaacctcagcatccagagatacgctgtcatccctttatcgaccaactcgggcctcattggctgggttccccactgtgacacactgcacgccctcatccgggactacagggagaagaagaagatccttctcaacatcgagcatcgcatcatgttgcggatggctccggactatgaccacttgactctgatgcagaaggtggaggtgtttgagcatgccgtcaataatacagctggggacgacctggccaagctgctgtggctgaaaagccccagctccgaggtgtggtttgaccgaagaaccaattatacccgttctttagcggtcatgtcaatggttgggtatattttaggcctgggagatagacacccatccaacctgatgctggaccgtctgagtgggaagatcctgcacattgactttggggactgctttgaggttgctatgacccgagagaagtttccagagaagattccatttagactaacaagaatgttgaccaatgctatggaggttacaggcctggatggcaactacagaatcacatgccacacagtgatggaggtgctgcgagagcacaaggacagtgtcatggccgtgctggaagcctttgtctatgaccccttgctgaactggaggctgatggacacaaataccaaaggcaacaagcgatcccgaacgaggacggattcctactctgctggccagtcagtcgaaattttggacggtgtggaacttggagagccagcccataagaaaacggggaccacagtgccagaatctattcattctttcattggagacggtttggtgaaaccagaggccctaaataagaaagctatccagattattaacagggttcgagataagctcactggtcgggacttctctcatgatgacactttggatgttccaacgcaagttgagctgctcatcaaacaagcgacatcccatgaaaacctctgccagtgctatattggctggtgccctttctggtaactggaggcccagatgtgcccatcacgttttttctgaggcttttgtactttagtaaatgcttccactaaactgaaaccatggtgagaaagtttgactttgttaaatattttgaaatgtaaatgaaaagaactactgtatattaaaagttggtttgaaccaactttctagctgctgttgaagaatatattgtcagaaacacaaggcttgatttggttcccaggacagtgaaacatagtaataccacgtaaatcaagccattcattttggggaacagaagatccataactttagaaatacgggttttgacttaactcacaagagaactcatcataagtacttgctgatggaagaatgacctagttgctcctctcaacatgggtacagcaaactcagcacagccaagaagcctcaggtcgtggagaacatggattaggatcctagactgtaaagacacagaagatgctgacctcacccctgccacctatcccaagacctcactggtctgtggacagcagcagaaatgtttgcaagataggccaaaatgagtacaaaaggtctgtcttccatcagacccagtgatgctgcgactcacacgcttcaattcaagacctgaccgctagtagggaggtttattcagatcgctggcagcctcggctgagcagatgcacagaggggatcactgtgcagtgggaccaccctcactggccttctgcagcagggttctgggatgttttcagtggtcaaaatactctgtttagagcaagggctcagaaaacagaaatactgtcatggaggtgctgaacacagggaaggtctggtacatattggaaattatgagcagaacaaatactcaactaaatgcacaaagtataaagtgtagccatgtctagacaccatgttgtatcagaataatttttgtgccaataaatgacatcagaattttaaacatatgtaaaaaaaaa

SEQ ID NO: 7 - protein, 655 aa, Homo sapiens
MAEAPQVVEIDPDFEPLPRPRSCTWPLPRPEFSQSNSATSSPAPSGSAAANPDAAAGLPSASAAAVSADFMSNLSLLEESEDFPQAPGSVAAAVAAAAAAAATGGLCGDFQGPEAGCLHPAPPQPPPPGPLSQHPPVPPAAAGPLAGQPRKSSSSRRNAWGNLSYADLITKAIESSAEKRLTLSQIYEWMVKSVPYFKDKGDSNSSAGWKNSIRHNLSLHSKFIRVQNEGTGKSSWWMLNPEGGKSGKSPRRRAASMDNNSKFAKSRSRAAKKKASLQSGQEGAGDSPGSQFSKWPASPGSHSNDDFDNWSTFRPRTSSNASTISGRLSPIMTEQDDLGEGDVHSMVYPPSAAKMASTLPSLSEISNPENMENLLDNLNLLSSPTSLTVSTQSSPGTMMQQTPCYSFAPPNTSLNSPSPNYQKYTYGQSSMSPLPQMPIQTLQDNKSSYGGMSQYNCAPGLLKELLTSDSPPHNDIMTPVDPGVAQPNSRVLGQNVMMGPNSVMSTYGSQASHNKMMNPSSHTHPGHAQQTSAVNGRPLPHTVSTMPHTSGMNRLTQVKTPVQVPLPHPMQMSALGGYSSVSSCNGYGRMGLLHQEKLPSDLDGMFIERLDCDMESIIRNDLMDGDTLDFNFDNVLPNQSFPHSVKTTTHSWVSG

SEQ ID NO: 8 - DNA, 5738 bp, Homo sapiens
gcagccgccacattcaacaggcagcagcgcagcgggcgcgccgctggggagagcaagcggcccgcggcgtccgtccgtccttccgtccgcggccctgtcagctggagcgcggcgcaggctctgccccggcccggcggctctggccggccgtccagtccgtgcggcggaccccgaggagcctcgatgtggatggccccgcgaagttaagttctgggctcgcgcttccactccgccgcgccttcctcccagtttccgtccgctcgccgcaccggcttcgttcccccaaatctcggaccgtcccttcgcgccccctccccgtccgcccccagtgctgcgttctccccctcttggctctcctgcggctgggggaggggcgggggtcaccatggccgaggcgcctcaggtggtggagatcgacccggacttcgagccgctgccccggccgcgctcgtgcacctggccgctgcccaggccggagtttagccagtccaactcggccacctccagcccggcgccgtcgggcagcgcggctgccaaccccgacgccgcggcgggcctgccctcggcctcggctgccgctgtcagcgccgacttcatgagcaacctgagcttgctggaggagagcgaggacttcccgcaggcgcccggctccgtggcggcggcggtggcggcggcggccgccgcggccgccaccggggggctgtgcggggacttccagggcccggaggcgggctgcctgcacccagcgccaccgcagcccccgccgcccgggccgctgtcgcagcacccgccggtgccccccgccgccgctgggccgctcgcggggcagccgcgcaagagcagctcgtcccgccgcaacgcgtggggcaacctgtcctacgccgacctcatcaccaaggccatcgagagctcggcggagaagcggctcacgctgtcgcagatctacgagtggatggtcaagagcgtgccctacttcaaggataagggtgacagcaacagctcggcgggctggaagaattcaattcgtcataatctgtccctacacagcaagttcattcgtgtgcagaatgaaggaactggaaaaagttcttggtggatgctcaatccagagggtggcaagagcgggaaatctcctaggagaagagctgcatccatggacaacaacagtaaatttgctaagagccgaagccgagctgccaagaagaaagcatctctccagtctggccaggagggtgctggggacagccctggatcacagttttccaaatggcctgcaagccctggctctcacagcaatgatgactttgataactggagtacatttcgccctcgaactagctcaaatgctagtactattagtgggagactctcacccattatgaccgaacaggatgatcttggagaaggggatgtgcattctatggtgtacccgccatctgccgcaaagatggcctctactttacccagtctgtctgagataagcaatcccgaaaacatggaaaatcttttggataatctcaaccttctctcatcaccaacatcattaactgtttcgacccagtcctcacctggcaccatgatgcagcagacgccgtgctactcgtttgcgccaccaaacaccagtttgaattcacccagcccaaactaccaaaaatatacatatggccaatccagcatgagccctttgccccagatgcctatacaaacacttcaggacaataagtcgagttatggaggtatgagtcagtataactgtgcgcctggactcttgaaggagttgctgacttctgactctcctccccataatgacattatgacaccagttgatcctggggtagcccagcccaacagccgggttctgggccagaacgtcatgatgggccctaattcggtcatgtcaacctatggcagccaggcatctcataacaaaatgatgaatcccagctcccatacccaccctggacatgctcagcagacatctgcagttaacgggcgtcccctgccccacacggtaagcaccatgccccacacctcgggtatgaaccgcctgacccaagtgaagacacctgtacaagtgcctctgccccaccccatgcagatgagtgccctggggggctactcctccgtgagcagctgcaatggctatggcagaatgggccttctccaccaggagaagctcccaagtgacttggatggcatgttcattgagcgcttagactgtgacatggaatccatcattcggaatgacctcatggatggagatacattggattttaactttgacaatgtgttgcccaaccaaagcttcccacacagtgtcaagacaacgacacatagctgggtgtcaggctgagggttagtgagcaggttacacttaaaagtacttcagattgtctgacagcaggaactgagagaagcagtccaaagatgtctttcaccaactcccttttagttttcttggttaaaaaaaaaaacaaaaaaaaaaaccctccttttttcctttcgtcagacttggcagcaaagacatttttcctgtacaggatgtttgcccaatgtgtgcaggttatgtgctgctgtagataaggactgtgccattggaaatttcattacaatgaagtgccaaactcactacaccatataattgcagaaaagattttcagatcctggtgtgctttcaagttttgtatataagcagtagatacagattgtatttgtgtgtgtttttggtttttctaaatatccaattggtccaaggaaagtttatactctttttgtaatactgtgatgggcctcatgtcttgataagttaaacttttgtttgtactacctgttttctgcggaactgacggatcacaaagaactgaatctccattctgcatctccattgaacagccttggacctgttcacgttgccacagaattcacatgagaaccaagtagcctgttatcaatctgctaaattaatggacttgttaaacttttggaaaaaaaaagattaaatgccagctttgtacaggtcttttctatttttttttgtttattttgttatttgcaaatttgtacaaacatttaaatggttctaatttccagataaatgatttttgatgttattgttgggacttaagaacatttttggaatagatattgaactgtaataatgttttcttaaaactagagtctactttgttacatagtcagcttgtaaattttgtggaaccacaggtatttggggcagcattcataattttcattttgtattctaactggattagtactaattttatacatgcttaactggtttgtacactttgggatgctacttagtgatgtttctgactaatcttaaatcattgtaattagtacttgcatattcaacgtttcaggccctggttgggcaggaaagtgatgtatagttatggacactttgcgtttcttatttaggataacttaatatgtttttatgtatgtattttaaagaaatttcatctgcttctactgaactatgcgtactgcatagcatcaagtcttctctagagacctctgtagtcctgggaggcctcataatgtttgtagatcagaaaagggagatctgcatctaaagcaatggtcctttgtcaaacgagggattttgatccacttcaccattttgagttgagctttagcaaaagtttcccctcataattctttgctcttgtttcagtccaggtggaggttggttttgtagttctgccttgaggaattatgtcaacactcatacttcatctcattctcccttctgccctgcagattagattacttagcacactgtggaagtttaagtggaaggagggaatttaaaaatgggacttgagtggtttgtagaatttgtgttcataagttcagatgggtagcaaatggaatagaacttacttaaaaattggggagatttatttgaaaaccagctgtaagttgtgcattgagattatgttaaaagccttggcttaagaatttgaaaatttctttagcctgtagcaacctaaactgtaattcctatcattatgttttattactttccaattacctgtaactgacagaccaaattaattggctttgtgtcctatttagtccatcagtattttcaagtcatgtggaaagcccaaagtcatcacaatgaagagaacaggtgcacagcactgttcctcttgtgttcttgagaaggatctaatttttctgtatatagcccacatcacacttgctttgtcttgtatgttaattgcatcttcattggcttggtatttcctaaatgtttaacaagaacacaagtgttcctgataagatttcctacagtaagccagctctattgtaagcttcccactgtgatgatcatttttttgaagattcattgaacagccaccactctatcatcctcattttggggcagtccaagacatagctggttttagaaacccaagttcctctaagcacagcctcccgggtatgtaactgaacttggtgccaaagtacttgtgtactaatttctattactacgtactgtcactttcctcccgtgccattactgcatcataatacaaggaacctcagagcccccatttgttcattaaagaggcaactacagccaaaatcactgttaaaatcttactacttcatggagtagctcttaggaaaatatatcttcctcctgagtctgggtaattatacctctcccaagcccccattgtgtgttgaaatcctgtcatgaatccttggtagctctctgagaacagtgaagtccagggaaaggcatctggtctgtctggaaagcaaacattatgtggcctctggtagtttttttcctgtaagaatactgactttctggagtaatgagtatatatcagttattgtacatgattgctttgtgaaatgtgcaaatgatatcacctatgcagccttgtttgatttattttctctggtttgtactgttattaaaagcatattgtattatagagctattcagatattttaaatataaagatgtattgtttccgtaatatagacgtatggaatatatttaggtaatagatgtattacttggaaagttctgctttgacaaactgacaaagtctaaatgagcacatgtatcccagtgagcagtaaatcaatggaacatcccaagaagaggataaggatgcttaaaatggaaatcattctccaacgatatacaaattggacttgttcaactgctggatatatgctaccaataaccccagccccaacttaaaattcttacattcaagctcctaagagttcttaatttataactaattttaaaagagaagtttcttttctggttttagtttgggaataatcattcattaaaaaaaatgtattgtggtttatgcgaacagaccaacctggcattacagttggcctctccttgaggtgggcacagcctggcagtgtggccaggggtggccatgtaagtcccatcaggacgtagtcatgcctcctgcatttcgctacccgagtttagtaacagtgcagattccacgttcttgttccgatactctgagaagtgcctgatgttgatgtacttacagacacaagaacaatctttgctataattgtataaagccataaatgtacataaattatgtttaaatggcttggtgtctttcttttctaattatgcagaataagctctttattaggaattttttgtgaagctattaaatacttgagttaagtcttgtcagccacaa

SEQ ID NO: 9 - protein, 673 aa, Homo sapiens
MAEAPASPAPLSPLEVELDPEFEPQSRPRSCTWPLQRPELQASPAKPSGETAADSMIPEEEDDEDDEDGGGRAGSAMAIGGGGGSGTLGSGLLLEDSARVLAPGGQDPGSGPATAAGGLSGGTQALLQPQQPLPPPQPGAAGGSGQPRKCSSRRNAWGNLSYADLITRAIESSPDKRLTLSQIYEWMVRCVPYFKDKGDSNSSAGWKNSIRHNLSLHSRFMRVQNEGTGKSSWWIINPDGGKSGKAPRRRAVSMDNSNKYTKSRGRAAKKKAALQTAPESADDSPSQLSKWPGSPTSRSSDELDAWTDFRSRTNSNASTVSGRLSPIMASTELDEVQDDDAPLSPMLYSSSASLSPSVSKPCTVELPRLTDMAGTMNLNDGLTENLMDDLLDNITLPPSQPSPTGGLMQRSSSFPYTTKGSGLGSPTSSFNSTVFGPSSLNSLRQSPMQTIQENKPATFSSMSHYGNQTLQDLLTSDSLSHSDVMMTQSDPLMSQASTAVSAQNSRRNVMLRNDPMMSFAAQPNQGSLVNQNLLHHQHQTQGALGGSRALSNSVSNMGLSESSSLGSAKHQQQSPVSQSMQTLSDSLSGSSLYSTSANLPVMGHEKFPSDLDLDMFNGSLECDMESIIRSELMDADGLDFNFDSLISTQNVVGLNVGNFTGAKQASSQSWVPG

SEQ ID NO: 10 - DNA, 7341 bp, Homo sapiens
gcgcgaggccgtcgattcgctcgcggctccatcgcggcctggccggggggcggtgtctgctgcgccaggttcgctggccgcacgtcttcaggtcctcctgttcctgggaggcgggcgcggcaggactgggaggtggcggcagcgggcgaggactcgccgaggacggggctccggcccgggataaccaactctccttctctcttctttggtgcttccccaggcggcggcggcggcgcccgggagccggagccttcgcggcgtccacgtccctcccccgctgcaccccgccccggcgcgagaggagagcgcgagagccccagccgcgggcgggcgggcggcgaagatggcagaggcaccggcttccccggccccgctctctccgctcgaagtggagctggacccggagttcgagccccagagccgtccgcgatcctgtacgtggcccctgcaaaggccggagctccaagcgagccctgccaagccctcgggggagacggccgccgactccatgatccccgaggaggaggacgatgaagacgacgaggacggcgggggacgggccggctcggccatggcgatcggcggcggcggcgggagcggcacgctgggctccgggctgctccttgaggactcggcccgggtgctggcacccggagggcaagaccccgggtctgggccagccaccgcggcgggcgggctgagcgggggtacacaggcgctgctgcagcctcagcaaccgctgccaccgccgcagccgggggcggctgggggctccgggcagccgaggaaatgttcgtcgcggcggaacgcctggggaaacctgtcctacgcggacctgatcacccgcgccatcgagagctccccggacaaacggctcactctgtcccagatctacgagtggatggtgcgttgcgtgccctacttcaaggataagggcgacagcaacagctctgccggctggaagaactccatccggcacaacctgtcactgcatagtcgattcatgcgggtccagaatgagggaactggcaagagctcttggtggatcatcaaccctgatggggggaagagcggaaaagccccccggcggcgggctgtctccatggacaatagcaacaagtataccaagagccgtggccgcgcagccaagaagaaggcagccctgcagacagcccccgaatcagctgacgacagtccctcccagctctccaagtggcctggcagccccacgtcacgcagcagtgatgagctggatgcgtggacggacttccgttcacgcaccaattctaacgccagcacagtcagtggccgcctgtcgcccatcatggcaagcacagagttggatgaagtccaggacgatgatgcgcctctctcgcccatgctctacagcagctcagccagcctgtcaccttcagtaagcaagccgtgcacggtggaactgccacggctgactgatatggcaggcaccatgaatctgaatgatgggctgactgaaaacctcatggacgacctgctggataacatcacgctcccgccatcccagccatcgcccactgggggactcatgcagcggagctctagcttcccgtataccaccaagggctcgggcctgggctccccaaccagctcctttaacagcacggtgttcggaccttcatctctgaactccctacgccagtctcccatgcagaccatccaagagaacaagccagctaccttctcttccatgtcacactatggtaaccagacactccaggacctgctcacttcggactcacttagccacagcgatgtcatgatgacacagtcggaccccttgatgtctcaggccagcaccgctgtgtctgcccagaattcccgccggaacgtgatgcttcgcaatgatccgatgatgtcctttgctgcccagcctaaccagggaagtttggtcaatcagaacttgctccaccaccagcaccaaacccagggcgctcttggtggcagccgtgccttgtcgaattctgtcagcaacatgggcttgagtgagtccagcagccttgggtcagccaaacaccagcagcagtctcctgtcagccagtctatgcaaaccctctcggactctctctcaggctcctccttgtactcaactagtgcaaacctgcccgtcatgggccatgagaagttccccagcgacttggacctggacatgttcaatgggagcttggaatgtgacatggagtccattatccgtagtgaactcatggatgctgatgggttggattttaactttgattccctcatctccacacagaatgttgttggtttgaacgtggggaacttcactggtgctaagcaggcctcatctcagagctgggtgccaggctgaaggatcactgaggaaggggaagtgggcaaagcagaccctcaaactgacacaagacctacagagaaaaccctttgccaaatctgctctcagcaagtggacagtgataccgtttacagcttaacacctttgtgaatcccacgccattttcctaacccagcagagactgttaatggccccttaccctgggtgaagcacttacccttggaacagaactctaaaaagtatgcaaaatcttccttgtacagggtggtgagccgcctgccagtggaggacagcacccctcagcaccacccaccctcattcagagcacaccgtgagcccccgtcggccattctgtggtgttttaatattgcgatggtttatgggacgttttaagtgttgttcttgtgtttgttttcctttgactttctgagtttttcacatgcattaacttgcggtatttttctgttaaaatgttaaccgtccttcccctagcaaatttaaaaacagaaagaaaatgttgtaccagttaccattccgggttcgagcatcacaagcttttgagcgcatggaactccataaactaacaaattacataaactaaagggggattttctttcttcttttgtttggtagaaaattatccttttctaaaaactgaacaatggcacaattgtttgctatgtgcacccgtccaggacagaaccgtgcataggcaaaaggagtggagcacagcgtccggcccagtgtgtttccggttctgagtcagggtgatctgtggacgggaccccagcaccaagtctacgggtgccagatcagtagggcctgtgatttcctgtcagtgtcctcagctaatgtgaacagtgttggtctgctggttagaaactagaatattgatattttcaggaaagaaatcagctcagctctccactcattgccaaatgtcactaaagggtttagttttaaggagaaagaaaaggaaaaaaaaaaaaaacaaaaaagtcctgttttgctttgcagaacaaatgaacttacaggtgagcattaagcttgcagtgagaaatgtgcgaagagtaaaaacccaagtcaatgctgaggcagttctaacttcactgttttcctaaatacacatccttgattattttcagccttgctatataatctgatctgctagaagtgtatgagtgagaggcaatagcatacaaactgattttttaaatataagcttaggttgtaattgtacaagtgactcaatggaagtacaaaatagggcagttttaacttttttttctgcttctatggatttcattttgttgtgttttcaaaaagttatggtgctgtataggtgctttctgtttaacctggaaagtgtgattatattcgttaccttctttggtagacggaatagttgggaccacctttggtacataagaaattggtataacgatgctctgattagcacagtatatgcatacttctccaaagtgatatatgaagactcttttctttgcataaaaagcattaggcatataaatgtataaatatattttatcatgtacagtacaaaaatggaaccttatgcatgggccttaggaatacaggctagtatttcagcacagacttccctgcttgagttcttgctgatgcttgcaccgtgacagtgggcaccaacacagacgtgccacccaaccccctgcacacaccaccggccaccaggggcccccttgtgcgccttggctttataactcctctgggggtgatattggtggtgatcacagctcctagcataatgagagttccatttggtattgtcacacgtctcctgcctcgcttgggttgccatgtttgagcgatggccctgttgatttcaccctgccttttactgaatctgtaaattgttgtgcaattgtggttatagtagactgtagcacattgccttttctaaactgctacatgtttataatcttcatttttaaagtatgtgtaatttttttaagtatgtattctattcatatggtctgcttgtcagtgagccagacttgcttactatattcctttataataatgctagccacttcctggattctttagtaatgtgctgtatgcaagaactttccagtagcagtgaaggagggttgcctctccaagcttcctaagggatgctgccctgtgtggggatgcattgcagaggcactagtagcatgggggctagagtggggagcgagatgtaaaagggtggggggataggagaattccagagtgcttccagcattagggtcctgagaacttctgagttcagagaaacatgcaaagtgactaacaaaatagctacttacctttgcagttttacagaccctgggagctgctttgggagtgagaaaggcaaccctccaatgtgtttcaactttaaaatgttgaattcttttcagacatggtatctcatttattctccttttctagcgtttgttgaatttcaggcagaatgtcttacagaatgtcctagaaccagattatcatttaatctgaaacagctgaggaagggacagagaaggtacaagggcaaggcagcacaaaacagatcaggagaatgaagagggaatgctttggttttttgttttgttttgttttttctttttcaagtaactaaaacagcatctacatgtagagtgttgtggagagctgagaccagggtaaagtcaagtgcagcatcagtactgcgagacccaccagcccctggagagggtcagccgagaatctggtagtgaagcctgtctagggtcccggcaccctcaccctcagccacctgcagagaggccagggccccagagactagcctggttctgaagtgggcaggggtgctgccagagccctctgccccttatgttgagaccctgctttcaggacaggccagccgttggccaccatgtcacattctgagtgagtgtcacaggtccctaacaataattttctgatctggagcatatcagcagaatgcttagcctcaaggggcctggcagctgtaatgtttgatttatgatgagaactatccgaggccacccttggcctctaaataagctgctctagggagccgcctactttttgatgagaaattagaagagtacctaatgttgaaaacatgacatgcgctcttgggatctgctgttctctccagggctccagaacctgatacctgttaccaaagctaggaaagagctttatcacaagccttcactgtcctggcatgagaactggctgccaggctcagtgtaccccattaactgtgaatgaatctgagcttggtttcctttattgcttcctctgcaatatgattgctgaaacacattttaaaaattcagaagcttgtcactcctgttaatgggaggatcagtcacacatgtgtagtacaaggcggactttgtgtttgtttttggtgttaatttttagcattgtgtgtgttgcttccccaccctgaggagaggacaccatggcttactactcaggacaagtatgccccgctcagggtgtgatttcaggtggcttccaaacttgtacgcagtttaaagatggtggggacagactttgcctctacctagtgaaccccacttaaagaataaggagcatttgaatctcttggaaaaggccatgaagaataaagcagtcaaaaagaagtcctccatgttggtgccaaggacttgcgaggggaaataaaaatgttatccagcctgaccaacatggagaaaccccgtctccattaaaaatacaaaattagcctggcatggtggcgcatgcctgtaatcccagctactctggaggctgaggcaggagaatcgcttgaacccaggaggcggaggtcgcagtgagccgagatcatgccagtgcactccagcctgggtaacaagagtgaaactccgtgtcaaaaaaaaaaaaaaaatgttactcatcctctctgaaagcaaaaaggaaaccctaacagctctgaactctggttttatttttcttgctgtatttgggtgaacattgtatgattaggcataatgttaaaaaaaaaaatttttttttggtagaaatgcaatcaccagtaaagaggtacgaaaaagctagcctctctcagagaccggggaggcagagtactactagaggaagtgaagttctgatggaatcatgcctgtcaaatgaggtcttgaagcggatgcccaaataaaagagtatattttatctaaatcttaagtgggtaacattttatgcagtttaaatgaatggaatattttcctcttgtttagttgtatctgtttgtatttttctttgatgaatgattggtcatgaggcctcttgccacactccagaaatacgtgtgcggctgcttttaagaactatgtgtctggtcacttatttctctaaaattatctcattgcctggcaatcagtcttctcttgtatacttgtcctagcacattatgtacatgggaaatgtaaacaaatgtgaaggaggaccagaaaaattagttaatatttaaaaaaatgtattgtgcattttggcttcacatgtttaactttttttaagaaaaaagttgcatgaatggaaaaaaaaatctgtatacagtatctgtaaaaactatcttatctgtttcaattccttgctcatatcccatataatctagaactaaatatggtgtgtggccatatttaaacacctgagagtcaagcagttgagactttgatttgaagcacctcatccttctttcaatgcgaacactatcatatggcattcttactgaggattttgtctaaccatatgttgccatgaattaactctgccgcctttcttaaggatcaaaaccagtttgatttgggaatcttcccctttccaaatgaaatagagatgcagtacttaactttccttggtgtttgtagatattgccttgtgtattccacttaaaaccgtaatctagtttgtaaaagagatggtgacgcatgtaaataaagcatcagtgacactct

SEQ ID NO: 11 - protein, 505 aa, Homo sapiens
MDPGNENSATEAAAIIDLDPDFEPQSRPRSCTWPLPRPEIANQPSEPPEVEPDLGEKVHTEGRSEPILLPSRLPEPAGGPQPGILGAVTGPRKGGSRRNAWGNQSYAELISQAIESAPEKRLTLAQIYEWMVRTVPYFKDKGDSNSSAGWKNSIRHNLSLHSKFIKVHNEATGKSSWWMLNPEGGKSGKAPRRRAASMDSSSKLLRGRSKAPKKKPSVLPAPPEGATPTSPVGHFAKWSGSPCSRNREEADMWTTFRPRSSSNASSVSTRLSPLRPESEVLAEEIPASVSSYAGGVPPTLNEGLELLDGLNLTSSHSLLSRSGLSGFSLQHPGVTGPLHTYSSSLFSPAEGPLSAGEGCFSSSQALEALLTSDTPPPPADVLMTQVDPILSQAPTLLLLGGLPSSSKLATGVGLCPKPLEAPGPSSLVPTLSMIAPPPVMASAPIPKALGTPVLTPPTEAASQDRMPQDLDLDMYMENLECDMDNIISDLMDEGEGLDFNFEPDP

SEQ ID NO: 12 - DNA, 3365 bp, Homo sapiens
aaaagggggagggaactgcggctaaggagacgttcggtgatgggagcgcaatatatgaggggatacagtgcctcaggtttaaaagagcaggaagctgagtgagaggttgcagaaaaagtgtcttcgctcggcagaggttacaggtggcatctcagaaagagctttgaggctacaggctgtagtcgggaaggggatcggagaactgtgtgaagggacagcttagggactagcgtcctgggactagggggaagttcgcgactttctgaagactggcaggaatgtgcctcctggccctcgatgcttcccccctgaggggaggcatcgtgagggactgtggcaggcttcactgaacgctgagccggggaggtccaactccacgtatggatccggggaatgagaattcagccacagaggctgccgcgatcatagacctagatcccgacttcgaaccccagagccgtccccgctcctgcacctggccccttccccgaccagagatcgctaaccagccgtccgagccgcccgaggtggagccagatctgggggaaaaggtacacacggaggggcgctcagagccgatcctgttgccctctcggctcccagagccggccgggggcccccagcccggaatcctgggggctgtaacaggtcctcggaagggaggctcccgccggaatgcctggggaaatcagtcatatgcagaactcatcagccaggccattgaaagcgccccggagaagcgactgacacttgcccagatctacgagtggatggtccgtactgtaccctacttcaaggacaagggtgacagcaacagctcagcaggatggaagaactcgatccgccacaacctgtccctgcacagcaagttcatcaaggttcacaacgaggccaccggcaaaagctcttggtggatgctgaaccctgagggaggcaagagcggcaaagccccccgccgccgggccgcctccatggatagcagcagcaagctgctccggggccgcagtaaagcccccaagaagaaaccatctgtgctgccagctccacccgaaggtgccactccaacgagccctgtcggccactttgccaagtggtcaggcagcccttgctctcgaaaccgtgaagaagccgatatgtggaccaccttccgtccacgaagcagttcaaatgccagcagtgtcagcacccggctgtcccccttgaggccagagtctgaggtgctggcggaggaaataccagcttcagtcagcagttatgcagggggtgtccctcccaccctcaatgaaggtctagagctgttagatgggctcaatctcacctcttcccattccctgctatctcggagtggtctctctggcttctctttgcagcatcctggggttaccggccccttacacacctacagcagctcccttttcagcccagcagaggggcccctgtcagcaggagaagggtgcttctccagctcccaggctctggaggccctgctcacctctgatacgccaccaccccctgctgacgtcctcatgacccaggtagatcccattctgtcccaggctccgactcttctgttgctgggggggcttccttcctccagtaagctggccacgggcgtcggcctgtgtcccaagcccctagaggctccaggccccagcagtctggttcccaccctttctatgatagcaccacctccagtcatggcaagtgcccccatccccaaggctctggggactcctgtgctcacaccccctactgaagctgcaagccaagacagaatgcctcaggatctagatcttgatatgtatatggagaacctggagtgtgacatggataacatcatcagtgacctcatggatgagggcgagggactggacttcaactttgagccagatccctgagtcatgcctggaagctttgtcccctgcttcagatgtggagccaggcgtgttcatatctactctttacccttgagccctccccaggaatttgggaccctgctttagagctagggtggggtctggtcacacacaggtgttgaagaaattataaagataaagctgccccatctggggacgatatggggagggagatgggaggggaaaggggagagggtttttctcactgtgccaattagggggtaaggccccctctcaggagccatcatcggctttccccattcctacccacttaggctttgtagcaagatgagcaatgctgttggaaatgtgaagtcaccagtggccttacccctgcctttgggagcaggatttttttgtagagagtcttatctgagctgagccaggctagctggagcctgggatttctatgcagtggccccttaggccagtgatgtgcggtgggtgggctgtttaggggatctggaagggccaaggtctgagcactggagtggctcgccaggccaaatcacccttagaaggctgcagataacagaaaggctttttataaacttttaaagaaatataaacacaaatatagagattttttaaccatggcagggtgctagtggtgggcagaatgcttttttttctttctgaaggctttgtgatagtgacatgatacaaacactacagacaataaatattaggagacacagggaagtggggagaggtggggagtaatagtaaacacagggaagagctcccctacggaccaggtatagagaaaggtctatgcagaaataggttagagtttccctaacaaaaaagctaacccaggtcccctcattccttcaacttgtgcctgggagtgtgtggtgttagggtgcagccacactcttctatgacccagcatgggttagtgctatggtgggagagtacattgaaggcctggaattagcttggggccagggaagggactgggaggggagagaagagaaggagggaaggatttaggatggtaaagttaggtacagagacctccctgttcaaggcccctgacagctgtccctgcccttcttccccttccctgactgcaggggttatgtggaagtgtgtgtggcagcaggcagcggggaggggaggaacagggaagggggagctggggagcttggctgagggtctgggaaatgagcagggatggggggggatgtggatcaggtttactagcacctgccagggaggccatctggggctccttctccaccccagcccccaaagcagcccttcccccagtgccctttgcatcgtcccctcccccacccctgctgtgggttcccatcatttcctgtgtcagcgcctggcctacccagattgtatcatgtgctagattggagtggggaagtgtgtcaaatcaataaatgaataaattcaataaatgcctataaccagcaaaaaaaaaaaaaaaaa

SEQ ID NO: 13 - protein, 573 aa, Homo sapiens
MVQSGLVPGEQGPCAGPAPGERRQRRASSPLPPRLGPWRPAAETAPPGTPPAGLPGACRSRSRPPTLREASLPSSPEPGGTMAAKLRAHQVDVDPDFAPQSRPRSCTWPLPQPDLAGDEDGALGAGVAEGAEDCGPERRATAPAMAPAPPLGAEVGPLRKAKSSRRNAWGNLSYADLITKAIESAPDKRLTLSQIYDWMVRYVPYFKDKGDSNSSAGWKNSIRHNLSLHTRFIRVQNEGTGKSSWWMLNPEGGKTGKTPRRRAVSMDNGAKFLRIKGKASKKKQLQAPERSPDDSSPSAPAPGPVPAAAKWAASPASHASDDYEAWADFRGGGRPLLGEAAELEDDEALEALAPSSPLMYPSPASALSPALGSRCPGELPRLAELGGPLGLHGGGGAGLPEGLLDGAQDAYGPRPAPRPGPVLGAPGELALAGAAAAYPGKGAAPYAPPAPSRSALAHPISLMTLPGEAGAAGLAPPGHAAAFGGPPGGLLLDALPGPYAAAAAGPLGAAPDRFPADLDLDMFSGSLECDVESIILNDFMDSDEMDFNFDSALPPPPPGLAGAPPPNQSWVPG

SEQ ID NO: 14 - DNA, 1722 bp, Homo sapiens
atggtgcaatccggcctcgtcccgggagagcagggcccgtgcgcagggccggcgcccggggagcggcggcagcggcgcgcgtccagccccctgcctccgcgcctcggcccatggcgccccgcggctgagacagcgccgccagggacccctcccgcgggcctccccggggcgtgcagatcccggagccgcccgcccaccctccgcgaagcctccctcccctcctcgcccgagccgggcgggaccatggctgcgaagctgcgagcgcatcaggtggacgtggacccggacttcgcgccgcagagccggccgcgctcgtgtacctggcccctgccgcagcctgacttggccggcgacgaggacggagcgctgggcgcaggggtggccgagggcgccgaggactgcgggccggagcgccgggctacggccccggcgatggccccagcgccgcccctgggcgcggaggtcggaccgctgcggaaagcgaagagctctcggcggaacgcgtgggggaacctgtcctacgccgacctcatcaccaaagccatcgagagcgccccggacaagcggctcacgctctcgcagatctacgactggatggtccgttacgtgccctacttcaaggataaaggcgacagcaacagctcggccggctggaagaactccatccggcacaacctgtcgctgcacacccgtttcatccgcgtgcagaacgagggcaccggcaagagttcgtggtggatgctgaaccccgagggcggaaagacagggaagaccccgcggcgcagggccgtgtccatggacaacggggccaagttcctgcgcatcaagggcaaggcgagcaagaagaagcagctgcaggcgcccgagcgaagcccggacgacagctccccgagtgcgcccgccccggggccggtgcctgccgcagccaagtgggccgccagccccgcctcgcacgccagcgacgactacgaggcttgggccgacttccgcggcggcgggagacccctgctcggggaggcggccgagctggaggacgacgaggccctggaggccctggcgccatcatcgccgctcatgtacccaagccccgccagcgcgctgtcgccggcgctgggctcgcgctgtccgggtgagctgccccgcctggccgagctgggaggcccgctgggcctgcacggcggcggcggcgcggggctgcccgagggcctgctggacggcgcgcaggacgcgtacgggccgcggcccgcgcccaggcccggcccggtgctgggtgcgccgggggagctggcgctggcgggcgcagccgccgcctaccccggcaaaggggcggccccgtacgcgccgcccgcgccctcgcgcagtgccttagcccaccccatcagccttatgacgctgcccggcgaggcgggcgccgcgggcctggcaccgccgggccacgccgccgccttcgggggcccgcccggcggcctcctgctggacgctctgccggggccctacgctgccgccgccgccgggccgctgggcgccgcgcccgaccgcttcccggccgacctggacctcgacatgttcagcgggagcctcgagtgcgacgtggagtccatcatcctcaacgacttcatggacagcgacgaaatggacttcaacttcgattcggccctgcctccgccgccgccgggcctggccggggccccgccccccaaccagagctgggtgccgggctga

SEQ ID NO: 15 - DNA, 34 bp, Artificial Sequence
  Description of Artificial Sequence Synthetic primer
aattctcgagagtcaaagagtagctgttcatgaa

SEQ ID NO: 16 - DNA, 34 bp, Artificial Sequence
  Description of Artificial Sequence Synthetic primer
aattaagcttctaaaagcaaaatgagagagagga

SEQ ID NO: 17 - DNA, 23 bp, Artificial Sequence
  Description of Artificial Sequence Synthetic primer
acacgaagcaatgttttgtttta

SEQ ID NO: 18 - DNA, 20 bp, Artificial Sequence
  Description of Artificial Sequence Synthetic primer
aaggaagtctccccttcacc

SEQ ID NO: 19 - DNA, 20 bp, Artificial Sequence
  Description of Artificial Sequence Synthetic primer
ctccagatggcaaacatacg

SEQ ID NO: 20 - DNA, 20 bp, Artificial Sequence
  Description of Artificial Sequence Synthetic primer
tctgtgtgcagaaagactcg

SEQ ID NO: 21 - DNA, 20 bp, Artificial Sequence
  Description of Artificial Sequence Synthetic primer
gcacagtcaaggccgagaat

SEQ ID NO: 22 - DNA, 20 bp, Artificial Sequence
  Description of Artificial Sequence Synthetic primer
gccttctccatggtggtgaa

SEQ ID NO: 23 - DNA, 23 bp, Artificial Sequence
  Description of Artificial Sequence Synthetic primer
aactccttcaacctgtttttccc

SEQ ID NO: 24 - DNA, 21 bp, Artificial Sequence
  Description of Artificial Sequence Synthetic primer
tgcagcatgtactttggagca

SEQ ID NO: 25 - DNA, 21 bp, Artificial Sequence
  Description of Artificial Sequence Synthetic primer
gctgcgctatctcatccaaga

SEQ ID NO: 26 - DNA, 22 bp, Artificial Sequence
  Description of Artificial Sequence Synthetic primer
gggttctgaagtgctagttcac

SEQ ID NO: 27 - DNA, 21 bp, Artificial Sequence
  Description of Artificial Sequence Synthetic primer
acagttggaaaagtggcacaa

SEQ ID NO: 28 - DNA, 22 bp, Artificial Sequence
  Description of Artificial Sequence Synthetic primer
gcgacgaacgtagttatcacca